(19) United States
(12) Patent Application Publication
(10) Pub. No.: US 2009/0093687 A1
(43) Pub. Date: Apr. 9, 2009

(54) SYSTEMS AND METHODS FOR DETERMINING A PHYSIOLOGICAL CONDITION USING AN ACOUSTIC MONITOR

(76) Inventors: Valery G. Telfort, Montreal (CA); Victor F. Lanzo, Laval (CA); James P. Welch, Mission Viejo, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/044,883

(22) Filed: Mar. 7, 2008

Related U.S. Application Data
(60) Provisional application No. 60/893,853, filed on Mar. 8, 2007.
(60) Provisional application No. 60/893,858, filed on Mar. 8, 2007.
(60) Provisional application No. 60/893,850, filed on Mar. 8, 2007.
(60) Provisional application No. 60/893,856, filed on Mar. 8, 2007.

(51) Int. Cl. A61B 5/00

(52) U.S. Cl. 600/300

(57) ABSTRACT

A method of communicating with a physiological sensor is disclosed. In an embodiment, the method includes supplying power through a first conductor in a first mode to the physiological sensor and communicating with an information element through the first conductor in a second mode. The physiological sensor includes the information element, a power supply configured to receive and store power from the first conductor in the first mode, and sensing circuitry configured to receive power from the first conductor in the first mode. The power supply releases the stored power to the sensing circuitry in the second mode.

### CROSS-REFERENCE TO RELATED APPLICATIONS

This application claims priority from U.S. Provisional Application No. 60/893,853, filed Mar. 8, 2007, titled “Multi-Parameter Physiological Monitor,” which is incorporated herein by reference in its entirety. This application also claims priority from U.S. Provisional Application No. 60/893,858, filed Mar. 8, 2007, titled “Multi-Parameter Sensor For Physiological Monitoring,” which is also incorporated herein by reference in its entirety. This application also claims priority from U.S. Provisional Application No. 60/893,850, filed Mar. 8, 2007, titled “Backward Compatible Physiological Sensor with Information Element,” which is also incorporated herein by reference in its entirety. This application also claims priority from U.S. Provisional Application No. 60/893,856, filed Mar. 8, 2007, titled “Physiological Monitor with Fast Gain Adjust Data Acquisition,” which is also incorporated herein by reference in its entirety.

### BACKGROUND

1. Field of the Invention

The present invention relates to systems and methods for determining a physiological condition using an acoustic monitor.

2. Description of the Related Art

Many life-threatening conditions are related to heart and respiratory failure. Heart disease, for instance, has become a leading cause of death, increasing the importance of the clinical technician's ability to recognize abnormal heart conditions. Likewise, continuous monitoring of respiratory activity is typically desirable in clinical situations, as death or brain damage can occur within minutes of respiratory failure. Appropriate heart and respiratory monitoring equipment can therefore be life-saving. Moreover, such equipment may also be useful for non-critical care, including exercise testing and different types of cardiac investigations.

One of the most powerful traditional techniques for non-invasive heart and respiratory monitoring is auscultation. Traditionally, auscultation is based on a physician's ability to use a stethoscope to recognize specific patterns and phenomena. In some cases, electronic listening equipment is used to hear the acoustic sounds (e.g., breathing, heart beating, etc.) generated within a patient.

Typical electronic listening equipment may include one or more sensors or transducers that obtains acoustic information from a patient and converts this information into a time-varying voltage signal. Some breathing and heart sounds are very small in magnitude, and the sensor will typically output very low voltages corresponding to these sounds. In order for a computer to properly process these voltages into useful information for diagnosis, these low voltages are often amplified.

One or more amplifiers are generally used to amplify the signal to a higher voltage level. The signal is then transmitted to an analog-to-digital converter which converts the signal into digital form. Thereafter, the signal is sent to a processor which manipulates the signal to obtain desired information about the patient.

However, due to the limitations of such equipment, certain sounds are difficult to measure. Many biological sounds are much louder than typical heart and breathing sounds. For instance, coughing, sneezing, snoring, wheezing, and speech can be several orders of magnitude louder than a patient's breathing and heart sounds. Amplifiers in electronic listening equipment are typically not calibrated to properly amplify or attenuate the high voltage sensor signals corresponding to these sounds.

Consequently, an amplifier will saturate at a certain voltage level, which is predetermined by the physical characteristics of the amplifier. Because this saturation level is less than the correct voltage level of the amplified signal, a portion of the signal will be lost. Loss of signal information due to a saturation event is referred to as “clipping” of the signal.

Signal clipping can have a detrimental impact on diagnosis and treatment of the patient. For some loud sounds, such as snoring, the sound of a patient breathing is overpowered largely by the loud sound. Prolonged loud sounds, such as snoring, may saturate the amplifier for extended periods of time. Therefore, much data about a patient's breathing pattern may be lost. In addition, data regarding heart sounds may be buried in the far louder sound of a snore, cough, wheeze, or other loud sound.

Some listening equipment compensates for these problems by including a manually-adjustable amplifier. In such devices, the gain of the amplifier is adjusted by a nurse or technician. When a patient begins to cough, the technician decreases the gain of the amplifier to adjust the input voltage signal to a non-saturation region of the amplifier. However, if the technician is not available to make the gain adjustment, data will be lost. In addition, even if the technician makes the adjustment, data may be lost immediately before and during the adjustment period. Furthermore, the technician must initially calibrate the gain of the amplifier to properly amplify low-level voltage signals corresponding to low-volume sounds.

In addition to typical electronic listening equipment, multiple additional devices are generally provided to measure or detect additional physiological parameters of the patient. For example, a respirometer may be provided to measure respiratory signals of the patient; an echocardiogram may be provided to monitor the electrical activity of the patient's heart; a capnograph may be provided to measure carbon dioxide concentration in inspired and expired air; and a photoplethysmograph may be provided to monitor the concentration of oxygen or other analytes in the patient's blood.

Each device typically has its own sensor and processing system, and is often connected to multiple tissue sites on the patient. Some devices, such as electrocardiographs (ECG), have many sensors interfacing with a processing system. When more than one device is connected to a patient, there may be several sensors connected to the patient at one time. Setting up several sensors may require a significant amount of time and cause some discomfort to the patient. It would therefore be advantageous to be able to measure more than one physiological parameter of a patient with a single sensor, thereby reducing the number of sensors to be connected to the patient's body. In addition, multiple devices having unique processing systems may not be compatible with some devices or might require special adapters to interface with those devices.

### SUMMARY

In certain embodiments, a physiological monitor for generating an electrocardiograph signal indicative of the electrical activity of a patient's heart includes an acoustic sensor for detecting both acoustic biological information and electrical biological information from a medical patient. The acoustic sensor includes an acoustic signal line and an electrical ground line. The physiological monitor further includes an electrocardiograph sensor for detecting electrical biological information from a medical patient, and the electrocardiograph sensor includes an electrode. A signal decoupling circuit is coupled with the acoustic sensor, which provides an acoustic signal to a processor and electrically decouples the acoustic sensor from the electrocardiograph sensor. An electrocardiograph circuit coupled with the acoustic sensor and with the electrocardiograph sensor measures a voltage signal between the electrical ground line of the acoustic sensor and the electrode of the electrocardiograph sensor. The electrocardiograph circuit also generates an electrocardiograph signal including the voltage signal.

In another embodiment, the physiological monitor also includes a second signal decoupling circuit coupled with the electrocardiograph sensor. The second signal decoupling circuit is operative to provide an electrical signal to the processor and to electrically decouple the electrocardiograph sensor from the first acoustic sensor.

In another embodiment, the physiological monitor also includes a second acoustic sensor operative to detect both acoustic biological information and electrical biological information from a medical patient. The second acoustic sensor includes an acoustic signal line and an electrical ground line.

In another embodiment, the first signal decoupling circuit includes a decoupling selected from the group of a DC-DC converter and an optocoupler. In another embodiment, the physiological monitor also includes a transient voltage suppression device interposed between the first acoustic sensor and the electrocardiograph sensor.

In another embodiment, the physiological monitor also includes a power decoupling circuit in communication with a voltage source and with the first acoustic sensor. The power decoupling circuit is operative to electrically decouple the first acoustic sensor and the second acoustic sensor.

In another embodiment, the first acoustic sensor also includes a frame, a sensing element wrapped at least partially around the frame, and a printed circuit board positioned adjacent the sensing element such that the first portion and second portion contact one side of the printed circuit board. The sensing element includes a first face, a second face, and at least one though hole. The sensing element also includes a first conductive layer on the first face, inside the through hole, and on a first portion of the second face such that the first face and first portion are in electrical communication with each other. The sensing element also includes a second conductive layer on a second portion of the second face.

In another embodiment, the physiological monitor also includes a bonding layer positioned between the frame and the sensing element. The bonding layer substantially prevents moisture from entering an acoustic chamber defined by the frame, sensing element, and printed circuit board.

In another embodiment, the frame includes at least one contact bump configured to provide pressure between the first portion of the sensing element and a corresponding contact on the printed circuit board. In another embodiment, the physiological monitor also includes at least one locking post configured to securely hold the printed circuit board in contact with the sensing element. In another embodiment, the first voltage signal has a peak-to-peak value of about 1 millivolt.

In other implementations, a physiological monitor for generating an electrocardiograph signal indicative of the electrical activity of a patient's heart includes a first acoustic sensor operative to detect both acoustic biological information and electrical biological information from a medical patient. The first acoustic sensor includes an acoustic signal line and an electrical ground line. The physiological monitor also includes a second acoustic sensor operative to detect both acoustic biological information and electrical biological information from a medical patient, and the second acoustic sensor includes an acoustic signal line and an electrical ground line. A first signal decoupling circuit is coupled with the first acoustic sensor, which provides an acoustic signal to a processor and electrically decouples the first acoustic sensor from the second acoustic sensor. An electrocardiograph circuit is coupled with the first acoustic sensor and with the second acoustic sensor, which measures a voltage signal between the electrical ground line of the first acoustic sensor and the electrical ground line of the second acoustic sensor. The electrocardiograph circuit also generates an electrocardiograph signal which includes the first voltage signal.

In another embodiment, the first voltage signal has a peak-to-peak value of about 1 millivolt. In another embodiment, the physiological monitor also includes a power decoupling circuit in communication with a voltage source and with the first acoustic sensor. The power decoupling circuit is operative to electrically decouple the first acoustic sensor and the second acoustic sensor.

In another embodiment, the first acoustic sensor also includes a frame, a sensing element wrapped at least partially around the frame, and a printed circuit board positioned adjacent the sensing element such that the first portion and second portion contact one side of the printed circuit board. The sensing element includes a first face, a second face, and at least one though hole. The sensing element also a first conductive layer on the first face, inside the through hole, and on a first portion of the second face such that the first face and first portion are in electrical communication with each other. The sensing element also includes a second conductive layer on a second portion of the second face.

Various embodiments include a method of generating an electrocardiograph signal indicative of the electrical activity of a patient's heart includes receiving a first electrical signal from a first acoustic sensor coupled to a patient, receiving a second electrical signal from a second acoustic sensor coupled to the patient, and determining an electrocardiograph signal based at least in part upon a difference between the first and second electrical signals. In another embodiment, the method also includes electrically decoupling the acoustic sensor from the electrocardiograph sensor.

In another embodiment, the method also includes providing a multi-parameter sensor. The multi-parameter sensor includes a frame, a sensing element wrapped at least partially around the frame, and a printed circuit board positioned adjacent the sensing element such that the first portion and second portion contact one side of the printed circuit board. The sensing element includes a first face, a second face, and at least one though hole. The wherein sensing element also includes a first conductive layer on the first face, inside the through hole, and on a first portion of the second face such that the first face and first portion are in electrical communication with each other. The sensing element also includes a second conductive layer on a second portion of the second face. In another embodiment, the difference between the first and second electrical signals is a voltage having a peak-to-peak value of about 1 millivolt.

In other embodiments, a method of generating an electrocardiograph signal indicative of the electrical activity of a patient's heart includes receiving a first electrical signal from a first acoustic sensor coupled to a patient, receiving a second electrical signal from a second acoustic sensor coupled to the patient, and determining an electrocardiograph signal based at least in part upon a difference between the first and second electrical signals. In another embodiment, the method also includes electrically decoupling the first acoustic sensor from the second acoustic sensor.

In another embodiment, the method also includes providing a multi-parameter sensor. The multi-parameter sensor includes a frame, a sensing element wrapped at least partially around the frame, and a printed circuit board positioned adjacent the sensing element such that the first portion and second portion contact one side of the printed circuit board. The sensing element includes a first face, a second face, and at least one though hole. The sensing element also includes a first conductive layer on the first face, inside the through hole, and on a first portion of the second face such that the first face and first portion are in electrical communication with each other. The sensing element also includes a second conductive layer on a second portion of the second face;

In another embodiment, the difference between the first and second electrical signals is a voltage having a peak-to-peak value of about 1 millivolt. In other embodiments, a physiological monitor for generating an electrocardiograph signal indicative of the electrical activity of a patient's heart includes a means for receiving a first electrical signal from an acoustic sensor coupled to a patient, a means for receiving a second electrical signal from an electrocardiograph sensor coupled to the patient; and a means for determining an electrocardiograph signal based at least in part upon a difference between the first and second electrical signals. In another embodiment, the physiological monitor also includes a means for electrically decoupling the acoustic sensor from the electrocardiograph sensor.

An alternative method of noninvasive heart and respiratory monitoring involves using an acoustic respiratory monitor (“ARM”). An ARM generally includes one or more acoustic sensors or transducers that obtain acoustic information from a patient and transmits the information to a processor for analysis. Once analyzed the processor sends the data to an output device, such as, for example, a visual display or audio speaker, for communication to the user or to a second processor for further analysis.

The foregoing acoustic sensor is typically detachable from the ARM to allow for periodic replacement. Periodic replacement of the acoustic sensor, as with other patient monitoring sensors, such as, for example, pulse oximeter sensors, ECG sensors, or the like, is advantageous for a wide variety of reasons. For example, the sensor can become soiled, thereby possibly inhibiting sensor sensitivity or causing cross-patient contamination. Furthermore, the electronic circuitry in the sensor can become damaged, thereby causing sensor failure or inaccurate results. Moreover, the securing mechanism for the sensor, such as an adhesive, can begin to fail, resulting in improper positioning. Accordingly, periodic replacement of the sensor is an important aspect of maintaining a sterile, highly sensitive, accurate patient monitoring sensor.

In addition, when a sensor is replaced, the new sensor may have different properties and manufacturing tolerances which may affect the signal transmitted to the patient monitor. These differences often go undetected, possibly resulting in inaccurate results. However, a typical acoustic sensor is generally reliant on an operator for timely replacement of soiled, damaged, or otherwise overused sensors. In addition, an operator replacing a sensor may not appreciate the problems associated with using different types of sensors. This approach is problematic, not only from the standpoint of operator mistake or neglect, but also from the perspective of deliberate misuse for cost saving or other purposes. However, because acoustic sensing systems can be expensive to replace, many users will not replace their existing systems with newer or upgraded systems with better sensor monitoring capabilities.

Aspects of the present disclosure include a backward compatible physiological sensor with an information element for use in tracking sensor use and providing sensor compatibility information. A physiological sensor is disclosed which includes an information element. In an embodiment, the information element is accessible over the power line connecting the physiological sensor to a patient monitor. This allows a sensor with an information element to be backward compatible with existing patient monitoring systems. In an embodiment, in order to allow the physiological sensor to continue to operate while the information element is accessed over the power line, a power supply is provided to the sensor. In an embodiment, the existing patient monitoring systems are reconfigured, either in software or hardware, to access the information element on the physiological sensor.

In an embodiment, the physiological sensor is an acoustic sensor. In an embodiment, the acoustic sensor includes a sensing element, such as, for example, a piezoelectric device or other acoustic sensing device. The sensing element generates a voltage which is responsive to vibrations. The acoustic sensor also includes circuitry which transmits the voltage generated by the sensing device to a processor for processing. In an embodiment, the physiological sensor is one of a pulse oximetry sensor, an ECG sensor, a blood pressure sensor, or the like. In an embodiment, the sensor is a multi-variable sensor as described in U.S. Application No. 60/893,858, filed Mar. 8, 2007, Attorney Docket No. MCAN.016PR, entitled “Multi-Parameter Sensor for Physiological Monitoring,” which was incorporated by reference above.

In an embodiment, the information element is a memory device, such as, for example, an erasable programmable read-only memory (“EPROM”). “EPROM” as used herein includes its broad ordinary meaning known to one of skill in the art, including those devices commonly referred to as “EEPROM,” those devices commonly referred to as “EPROM,” and those types of electronic devices capable of retaining their contents even when no power is applied and/or those types of devices that are reprogrammable. In an embodiment, the information element is an impedance value associated with the sensor, such as, for example, a resistive value, an impedance value, or an inductive value.

In an embodiment, the information element includes sensor use information which provides information about the use of the sensor, including information regarding the expiration of the useful life of the sensor, such as, for example, the amount of time the sensor is in use, the number of patients who have used the sensor, the age of the sensor, or the like. In an embodiment, the information element includes information regarding the type an/or identification of the sensor associated with the information element, such as, for example, the manufacturer, the model number, the serial number the patient type (e.g., adult, child, etc.), or the like. In an embodiment, the information element includes manufacturing tolerances and sensing properties, such as, for example, acoustic sensitivity, voltage ranges, current ranges, gain frequency response, calibration information, or the like. In an embodiment, the sensor stores use information, such as, for example, use time, use temperature, information regarding current use, voltage use, age of the sensor, or the like. In an embodiment, the information element can store patient specific information, such as, for example, patient identification, patient age, weight, sex, etc.; the amount of time used on a specific patient; the patient specific problems discovered by the sensor; the user; or the like. In one embodiment, the information element stores information obtained by the sensor before a major event occurs. For example, if a heart attack is detected by the monitor, the information element can store the acoustic information sensed by the sensor for a period of time before the heart attack occurred. In this way, a user can latter review and analyze what the sensor picked up right before the major event occurred. In one embodiment, the monitor uses identification information stored on the sensor in order to keep track of which sensors have been attached to the monitor.

In an embodiment, the sensor's power supply stores power received from the power line while the power line supplies power. When the power line stops supplying power in order to communicate with the information element, the power supply releases the stored power to the sensing device and the sensing circuitry. This allows the sensing device and the sensing circuitry to continue to operate while the information element is accessed over the power line. In an embodiment, the power supply is a capacitor. In an embodiment, the power supply is a battery. In an embodiment, the power supply is a battery which does not receive power from the monitor power line, but comes fully charged from the manufacturer. In an embodiment, the power supply is a user replaceable battery.

In an embodiment, a physiological sensor obtains physiological information from a patient and transmits the information to a physiological monitor. The physiological sensor includes first and second conductors which provide first and second communication paths for communicating with the physiological monitor. The physiological sensor also includes a power supply which receives and stores power via the first conductor in a first mode and which releases the stored power in a second mode.

The physiological sensor also includes sensing circuitry which receives power from the first conductor in the first mode and which receives power from the power supply in the second mode. The sensing circuitry obtains and communicates physiological information to the physiological monitor through the second conductor in the first and second modes. The physiological sensor also includes an information element which communicates with the monitor through the first conductor in the second mode.

In an embodiment, the physiological sensor operates in two modes. The first mode corresponds to a power supply mode and the second mode corresponds to an information element communication mode. Other modes of operation are available in other embodiments.

In an embodiment, the physiological sensor includes three or more conductors. In an embodiment, at least one of the three or more conductors communicates a ground signal. In an embodiment the ground signal is a floating ground signal. In an embodiment two of the three or more conductors communicate physiological information from the sensing circuitry.

In an embodiment, the sensing circuitry includes acoustic monitoring circuitry. In an embodiment, the acoustic monitoring circuitry includes a piezoelectric element. In an embodiment, the acoustic monitoring circuitry includes one or more of an amplifier, a filter, or an electrostatic discharge circuit. In an embodiment, the sensing circuitry includes blood parameter monitoring circuitry. In an embodiment, the sensing circuitry includes ECG monitoring circuitry. In an embodiment, the sensing circuitry includes blood pressure monitoring circuitry.

In an embodiment, the power supply includes at least one capacitor. In an embodiment, the power supply includes two or more capacitors. In an embodiment where at least two capacitors are included, at least one of the at least two capacitors releases energy quickly. In an embodiment where at least two capacitors are included, at least one of the at least two capacitors releases energy slowly. In an embodiment where at least two capacitors are included, at least one of the at least two capacitors releases energy over a relatively short period of time. In an embodiment where at least two capacitors are included, at least one of the at least two capacitors releases energy over a relatively long period of time. In an embodiment the power supply includes a battery.

In an embodiment, the information element includes an EPROM. In an embodiment, the information element stores one or more of a sensor type, a manufacturer, a model number, a serial number, a patient type, manufacturing tolerances, acoustic sensitivity, voltage information, current information, gain, an expiration date, an age of the sensor, use information, or patient information.

In an embodiment, a method of communicating with a physiological sensor is disclosed. The method includes the steps of supplying power through a first conductor in a first mode to a physiological sensor, where the physiological sensor includes a power supply which receives and stores power from the first conductor in the first mode and sensing circuitry which receives power from the first conductor in the first mode; communicating with an information element through the first conductor in a second mode; and receiving physiological information through the second conductor in the first and second modes, where the power supply releases the stored power to the sensing circuitry in the second mode.

In an embodiment, communicating with the information element in the second mode further includes communicating with the information element using a communication protocol. In an embodiment, the communication protocol includes an I²C protocol. In an embodiment, communicating with the information element in the second mode includes reading information from the information element. In an embodiment, communicating with the information element in the second mode includes writing information to the information element.

In an embodiment, the sensing circuitry includes acoustic monitoring circuitry. In an embodiment, the acoustic monitoring circuitry includes a piezoelectric element. In an embodiment, the sensing circuitry includes blood parameter monitoring circuitry. In an embodiment, the sensing circuitry includes ECG monitoring circuitry. In an embodiment, the sensing circuitry includes blood pressure monitoring circuitry.

In other embodiments, a method of communicating with a physiological monitor using an attachment includes receiving power at a physiological monitor attachment from a physiological monitor via a conductive path during a first operating mode, receiving a communication request at the physiological monitor attachment from the physiological monitor via the conductive path to initiate a second operating mode, and communicating with the physiological monitor using the physiological monitor attachment via the conductive path during the second operating mode.

In another embodiment, the method also includes providing power to the physiological monitor attachment from a secondary internal power source. In another embodiment, the physiological monitor attachment includes a physiological sensor. In another embodiment, the physiological monitor attachment includes a cable. In another embodiment, communicating with the physiological monitor includes sending information stored in an information element. In another embodiment, communication with the physiological monitor includes receiving information from the physiological monitor.

In an embodiment, a system for allowing a physiological monitor to communicate with a physiological sensor is disclosed. The system includes a physiological sensor which obtains physiological information from a patient. The physiological sensor includes means for storing information, means for storing power, and means for obtaining physiological information. The system also includes a physiological monitor which receives the physiological information from the physiological sensor and analyzes the physiological information. The physiological monitor also sends the physiological information to a display device for display. The physiological sensor and the physiological monitor include at least two communication paths for communications between the physiological sensor and the physiological monitor as well as means for allowing the physiological monitor to communicate with the means for storing information included within the physiological sensor without providing a separate communication path for communicating with the means for storing information.

In certain embodiments, a physiological sensor is configured to sense a physiological parameter related to a patient and provide information related to the physiological parameter to a physiological monitor. The physiological sensor includes a power port, an information element, and a power supply. The power port is configured to electronically couple the physiological sensor to a physiological monitor and to receive electrical power from the physiological monitor during a first operational mode. The information element is configured to communicate with the physiological monitor via the power port during a second operational mode. The power supply is configured to receive electrical power from the power port during the first operational mode and to provide electrical power during the second operational mode.

In another embodiment, the physiological sensor also includes a sensing circuit configured to receive power from the power port during the first operational mode and from the power supply during the second operational mode.

In certain embodiments, a physiological sensor includes a sensing circuit configured to provide a signal indicative of a physiological condition to a physiological monitor, an information element configured to communicate stored information to the physiological monitor, and a secondary power supply configured to supply power to the sensing circuit when the information element communicates with the physiological element.

In certain embodiments, a physiological monitoring apparatus for processing signals indicative of a physiological parameter of a medical patient includes a first gain stage that receives an input signal and transmits a first output signal. A first ratio of the first output signal to the input signal includes a first gain value. A second gain stage receives the input signal and transmits a second output signal, and a second ratio of the second output signal to the input signal includes a second gain value. At least one sampling circuit is in communication with the first gain stage and with the second gain stage, which samples the first and second output signals and outputs corresponding first and second sampled outputs. A processor is in communication with the sampling circuit, which constructs a third output signal comprising selected samples from the first and second sampled outputs.

In another embodiment, the processor selects a sample from the first sampled output in response to detecting clipping in a sample of the second output signal. In another embodiment, the processor multiplies the sample from the first sampled output by a relative gain factor. In another embodiment, the relative gain factor is a ratio of the second gain value to the first gain value.

In another embodiment, the physiological monitoring apparatus also includes at least one digitally-controlled amplifier operative to receive input from the processor and to amplify the first or second output signal.

In another embodiment, the at least one digitally-controlled amplifier includes a digital-to-analog converter and an operational amplifier. In another embodiment, the second gain stage includes at least one operational amplifier. In another embodiment, the physiological monitoring apparatus also includes a phase compensation circuit operative to compensate for phase differences between the second output signal and the first output signal.

In another embodiment, the phase compensation circuit includes a low pass filter. In another embodiment, the phase compensation circuit maintains a constant phase delay between the first output signal and the second output signal. In another embodiment, the first gain value is substantially equal to 0 decibels (dB). In another embodiment, the physiological monitoring apparatus also includes an isolation circuit in communication with the at least one sampling circuit and with the processor.

In another embodiment, the physiological monitoring apparatus also includes at least one additional gain stage. Each at least one additional gain stage is operative to receive the input signal and to amplify the input signal into an output signal.

Various implementations include a method for processing signals indicative of a physiological parameter of a medical patient. The method includes receiving an input signal at a first gain stage and at a second gain stage, transmitting a first output signal from the first gain stage to a sampling circuit, where a first ratio of the first output signal to the input signal includes a first gain value, transmitting a second output signal from the second gain stage to the at least one sampling circuit, where a second ratio of the second output signal to the input signal includes a second gain value, sampling the first and second output signals, outputting corresponding first and second sampled outputs, and constructing a third output signal including samples selected from the first and second sampled outputs.

In another embodiment, constructing a third output signal includes detecting clipping in a sample of the second sampled output. In another embodiment, constructing a third output signal also includes selecting a corresponding sample from the first sampled output in response to detecting clipping in the sample of the second sampled output.

In another embodiment, constructing a third output signal also includes multiplying the corresponding sample from the first sampled output by a relative gain factor. In another embodiment, the relative gain factor is a ratio of the second gain value to the first gain value. In another embodiment, the method also includes maintaining a constant phase delay between the first output signal and the second output signal.

In some implementations, a physiological monitoring system for processing signals indicative of a physiological parameter of a medical patient includes a sensor operative to receive biological information from a patient and to generate a signal based upon the biological information. An adjustable gain bank including at least two gain stages operative to receive the signal and transmit output signals, where the gain stages each have a gain value such that each output signal from the gain stages is substantially equal to the input signal multiplied by the gain value of the associated gain stage. At least one sampling circuit is in communication with the gain stages, which samples the output signals from the gain stages and generates at least two sampled outputs. At least one analog-to-digital converter is in communication with the sampling circuit, which converts the sampled outputs into digital form. A processor is in communication with the analog-to-digital converter, which constructs a processor output signal including samples from one or more of the sampled outputs.

In another embodiment, the physiological monitoring system also includes a phase compensation circuit operative to compensate for phase differences between the output signals. In another embodiment, the phase compensation circuit maintains a constant phase delay between the output signals.

In certain implementations, a method for processing signals indicative of a physiological parameter of a medical patient includes receiving an input signal at a low gain stage and at a high gain stage, converting a first output signal from the low gain stage and a second output signal from the high gain stage into digital format, detecting a number of least significant bits (LSBs) in the second output signal that change with respect to time, and changing a gain of a digitally-controlled amplifier based upon whether the number of LSBs that change with respect to time is less than a lower threshold number.

In another embodiment, the method also includes changing the gain when the number of LSBs that change with respect to time is more than an upper threshold number.

In certain embodiments, a physiological monitoring apparatus for processing signals indicative of a physiological parameter of a medical patient includes means for receiving an input signal at a first gain stage and at a second gain stage, means for transmitting a first output signal from the first gain stage to a sampling circuit, means for transmitting a second output signal from the second gain stage to the at least one sampling circuit, means for sampling the first and second output signals, means for outputting corresponding first and second sampled outputs, and means for constructing a third output signal. A first ratio of the first output signal to the input signal includes a first gain value. A second ratio of the second output signal to the input signal includes a second gain value. The third output signal includes samples selected from the first and second sampled outputs.

In one embodiment, a multi-parameter sensor for sensing more than one physiological parameter of a medical patient includes a frame, a sensing element, and a printed circuit board. The sensing element is wrapped at least partially around the frame and includes a first face, a second face, and at least one though hole. The sensing element also includes a first conductive layer on the first face, inside the through hole, and on a first portion of the second face such that the first face and first portion are in electrical communication with each other. The sensing element also includes a second conductive layer on a second portion of the second face. The printed circuit board is positioned adjacent the sensing element such that the first portion and second portion contact one side of the printed circuit board.

In another embodiment, the multi-parameter sensor also includes a bonding layer positioned between the frame and the sensing element. In one embodiment, the frame, sensing element and printed circuit board define an acoustic chamber, wherein the bonding layer substantially prevents moisture from entering the acoustic chamber.

In another embodiment, the multi-parameter sensor frame includes at least one contact bump configured to provide pressure between the first portion of the sensing element and a corresponding contact on the printed circuit board. In another embodiment, the multi-parameter sensor also includes at least one locking post configured to securely hold the printed circuit board in contact with the sensing element. In other embodiments, the sensing element includes a piezoelectric material. In another embodiment, the first conductive layer includes silver.

In another embodiment, the multi-parameter sensor includes an information element in electrical communication with the printed circuit board, which can be positioned on the printed circuit board. In some embodiments, the frame includes a rounded edge and the sensing element is wrapped around the rounded edge. In yet other embodiments, the printed circuit board is pressed into the frame, which places the sensing element in tension. In some embodiments, the frame also includes a raised ridge having dimensions selected to control tension on the sensing element.

In another embodiment, the sensing element senses more than one physiological parameter of a medical patient when the multi-parameter sensor is connected to the patient, such as an acoustical parameter of the medical patient and/or an ECG or EKG parameter of the medical patient.

In yet another embodiment, a method of sensing more than one physiological parameter of a medical patient, includes providing a multi-parameter sensor and generating a signal indicative of more than one physiological parameter of the medical patient when the multi-parameter sensor is connected to the patient.

The multi-parameter sensor includes a frame, a sensing element, and a printed circuit board. In one embodiment, the sensing element is wrapped at least partially around the frame and includes a first face, a second face, and at least one though hole. The sensing element also includes a first conductive layer on the first face, inside the through hole, and on a first portion of the second face such that the first face and first portion are in electrical communication with each other. The sensing element also includes a second conductive layer on a second portion of the second face. The printed circuit board is positioned adjacent the sensing element such that the first portion and second portion contact one side of the printed circuit board.

In one embodiment, generating a signal indicative of more than one physiological parameter of the medical patient includes generating a signal indicative of an acoustic, ECG, and/or EKG parameter of the medical patient. In one embodiment, the signal includes a superposition of an acoustic signal and an ECG or EKG signal. In another embodiment, the multi-parameter sensor includes a piezoelectric sensor and can further include an ECG or EKG electrode, as well. In one embodiment, the multi-parameter sensor comprises an ECG or EKG electrode.

In yet another embodiment, a multi-parameter sensor for sensing more than one physiological parameter of a medical patient includes a frame, a sensing element, a bonding layer, and a printed circuit board. The sensing element is wrapped at least partially around the frame and includes a first face, a second face, a first conductive layer on the first face, and a second conductive layer on the second face.

The bonding layer is positioned between the frame and sensing element, and is configured to prevent current flow from the first conductive layer to the second conductive layer. The printed circuit board is positioned adjacent the sensing element such that the first portion and second portion contact one side of the printed circuit board.

### BRIEF DESCRIPTION OF THE DRAWINGS

FIG. 1A is a block diagram illustrating an embodiment of a physiological monitoring system;

FIG. 1B is a block diagram illustrating another embodiment of a physiological monitoring system;

FIG. 2A illustrates an embodiment of an acoustic sensor;

FIG. 2B is a block diagram illustrating another embodiment of a physiological monitoring system;

FIG. 2C illustrates an embodiment of an ECG sensor;

FIG. 2D illustrates an embodiment of a pulse oximetry sensor;

FIG. 3A is a schematic block diagram illustrating an embodiment of a power supply circuit;

FIG. 3B is a schematic block diagram illustrating an embodiment of a signal acquisition system;

FIG. 3C is a schematic block diagram illustrating an embodiment of a transient voltage suppression circuit;

FIG. 4 is a flowchart diagram illustrating an embodiment of a method of generating a biological signal;

FIG. 5A is a block diagram illustrating an embodiment of a signal processing and routing system;

FIG. 5B is a block diagram illustrating another embodiment of the signal processing and routing of FIG. 5A;

FIG. 5C is a block diagram illustrating a further embodiment of the signal processing and routing of FIG. 5A;

FIG. 5D is a block diagram illustrating yet another embodiment of the signal processing and routing of FIG. 5A;

FIG. 6 is a cross-sectional view illustrating an embodiment of a sensor sub-assembly;

FIG. 7 is a cross-section view illustrating an embodiment of a sensing element;

FIG. 8 is a cross-sectional view illustrating an embodiment of a frame of a sensor subassembly;

FIG. 9 illustrates an embodiment of a respiratory monitoring system;

FIG. 9A illustrates an embodiment of an acoustic sensor;

FIG. 9B illustrates an embodiment of a pulse oximeter sensor;

FIG. 9C illustrates an embodiment of an ECG sensor;

FIG. 10A illustrates an embodiment of a physiological monitoring system incorporating an information element in a physiological sensor which is accessible over a power line;

FIG. 10B illustrates an embodiment of a physiological monitoring system incorporating an information element in a cable which is accessible over a power line;

FIG. 10C illustrates a circuit diagram of an embodiment of a physiological monitoring system incorporating an information element accessible over a power line;

FIG. 11A illustrates a frequency response of an embodiment of a piezoelectric device;

FIG. 11B illustrates a circuit diagram of an embodiment of a piezoelectric circuit;

FIG. 11C illustrates a circuit diagram of an embodiment of a piezoelectric circuit with impedance compensation;

FIG. 12A illustrates a circuit diagram of an embodiment of an information element;

FIG. 12B illustrates a circuit diagram of a secondary power supply;

FIG. 12C illustrates a power supply response of a secondary power supply;

FIG. 13 illustrates a circuit diagram of a common voltage supply;

FIG. 14A illustrates a flowchart of an embodiment of a physiological monitor operation;

FIG. 14B illustrates a flowchart of an embodiment of a physiological sensor operation;

FIG. 14C illustrates a flowchart of an embodiment of an information element operation;

FIG. 15A illustrates a flowchart of an embodiment of a physiological monitoring system incorporating an information element accessible over a power line;

FIG. 15B illustrates a flowchart of another embodiment of a physiological monitoring system incorporating an information element accessible over a power line;

FIG. 16A illustrates a flowchart of an information element operation;

FIG. 16B illustrates a flowchart of an operation for communicating with an information element;

FIG. 17A illustrates a flowchart of another embodiment of an information element operation;

FIG. 17B illustrates a flowchart of another embodiment of an operation for communicating with an information element;

FIG. 18 is an exemplary block diagram showing a physiological monitoring system according to an embodiment of the present invention;

FIG. 19 is an exemplary block diagram showing further embodiments of the physiological monitoring system;

FIG. 20 is an exemplary block diagram showing further embodiments of the physiological monitoring system;

FIG. 21 is an exemplary schematic diagram showing a physiological monitoring system according to an embodiment of the present invention;

FIG. 22A is an exemplary amplitude plot diagram showing an amplitude plot in accordance with embodiments of the present invention;

FIG. 22B is an exemplary phase plot diagram showing a phase plot in accordance with embodiments of the present invention;

FIG. 23A is an exemplary amplitude plot diagram showing an amplitude plot in accordance with embodiments of the present invention;

FIG. 23B is an exemplary phase plot diagram showing a phase plot in accordance with embodiments of the present invention;

FIG. 24 is an exemplary amplitude plot diagram showing an amplitude plot in accordance with embodiments of the present invention;

FIG. 25 is an exemplary schematic diagram showing a digitally-controlled amplifier according to an embodiment of the present invention;

FIG. 26 is an exemplary block diagram showing another embodiment of a physiological monitoring system;

FIG. 27 is an exemplary flowchart diagram showing a process for selecting samples according to an embodiment of the present invention;

FIG. 28 is an exemplary flowchart diagram showing a process for constructing a signal according to an embodiment of the present invention;

FIG. 29A is an exemplary signal diagram showing an analog signal in accordance with embodiments of the present invention;

FIG. 29B is an exemplary signal diagram showing another analog signal in accordance with embodiments of the present invention;

FIG. 29C is an exemplary signal diagram showing an amplified analog signal in accordance with embodiments of the present invention;

FIG. 29D is an exemplary signal diagram showing a sampled signal in accordance with embodiments of the present invention;

FIG. 29E is an exemplary signal diagram showing another sampled signal in accordance with embodiments of the present invention;

FIG. 29F is an exemplary signal diagram showing still another signal in accordance with embodiments of the present invention;

FIG. 30 is an exemplary flowchart diagram showing a process for calibrating a physiological monitoring system according to an embodiment of the present invention;

FIG. 31 is a top perspective view of a multi-parameter sensor assembly in accordance with one embodiment of the present invention;

FIG. 32 is a bottom perspective view of the multi-parameter sensor assembly of FIG. 31;

FIG. 33 is an exploded, top perspective view of the multi-parameter sensor assembly of FIGS. 31 and 32;

FIG. 34 is a top perspective view of a sensor subassembly of the multi-parameter sensor assembly of FIGS. 31-33;

FIG. 35 is a top perspective view of a frame of the sensor subassembly of FIG. 34;

FIG. 36 is a cross-sectional view of the frame of FIG. 35 taken along section line 36-36;

FIG. 37 is a top perspective view showing a bonding layer affixed to the frame of FIG. 35;

FIG. 38 is a top perspective view showing a sensing element affixed to the subassembly of FIG. 37;

FIG. 39 is a cross-sectional view taken along section line 39-39 of FIG. 38;

FIG. 40 is a top perspective view of the sensing element of FIGS. 38 and 39;

FIG. 41 is a cross-section view taken along section line 41-41 of the sensing element of FIG. 40;

FIG. 42 is a cross-section view of the sensing element of FIGS. 40 and 41 shown in a wrapped configuration;

FIG. 43 is a cross-sectional view taken along section line 43-43 of FIG. 34;

FIG. 44 is a cable assembly adapted to be removably coupled to the multi-parameter sensor of FIGS. 31-33;

FIG. 45 is a top perspective of a sensor system, which includes the multi-parameter sensor of FIGS. 31-33 and the cable assembly of FIG. 45; and

FIG. 46 is a block diagram of a physiological monitoring system, including a physiological monitor, and the sensor system of FIG. 46.

### DETAILED DESCRIPTION

Various embodiments according to the invention will be described hereinafter with reference to the accompanying drawings. These embodiments are illustrated and described by example only, and are not intended to limit the scope of the invention.

In various embodiments, a physiological monitoring system comprises or includes an acoustic signal processing system that measures and/or determines any of a variety of physiological parameters of a medical patient. For example, in an embodiment, the physiological monitoring system includes an acoustic respiratory monitor. An acoustic respiratory monitor can determine any of a variety of respiratory parameters of a patient, including respiratory rate, expiratory flow, tidal volume, minute volume, apnea duration, breath sounds, rales, rhonchi, stridor, and changes in breath sounds such as decreased volume or change in airflow. In addition, in some cases the acoustic signal processing system monitors other physiological sounds, such as heart rate to help with probe off detection, heart sounds (S1, S2, S3, S4, and murmurs), and change in heart sounds such as normal to murmur or split heart sounds indicating fluid overload. Moreover, the acoustic signal processing system may use a second probe over the chest for better heart sound detection, keep the user inputs to a minimum (example, height), and use a Health Level 7 (HL7) interface to automatically input patient demography.

In certain embodiments, the physiological monitoring system comprises or includes an electrocardiograph (ECG) that measures and/or determines electrical signals generated by the cardiac system of a patient. The ECG includes one or more sensors for measuring the electrical signals. In some embodiments, the electrical signals are obtained using the same sensors used to obtain acoustic signals.

In still other embodiments, the physiological monitoring system comprises or includes one or more additional sensors used to determine other desired physiological parameters. For example, in some embodiments, a photoplethysmograph sensor determines the concentrations of analytes contained in the patient's blood, such as oxyhemoglobin, carboxyhemoglobin, methemoglobin, other dyshemoglobins, total hemoglobin, fractional oxygen saturation, glucose, bilirubin, and/or other analytes. In other embodiments, a capnograph determines the carbon dioxide content in inspired and expired air from a patient. In other embodiments, other sensors determine blood pressure, pressure sensors, flow rate, air flow, and fluid flow (first derivative of pressure). Other sensors may include a pneumotachometer for measuring air flow and a respiratory effort belt. In certain embodiment, certain of these sensors are combined in a single processing system which processes signal output from the sensors on a single multi-function circuit board.

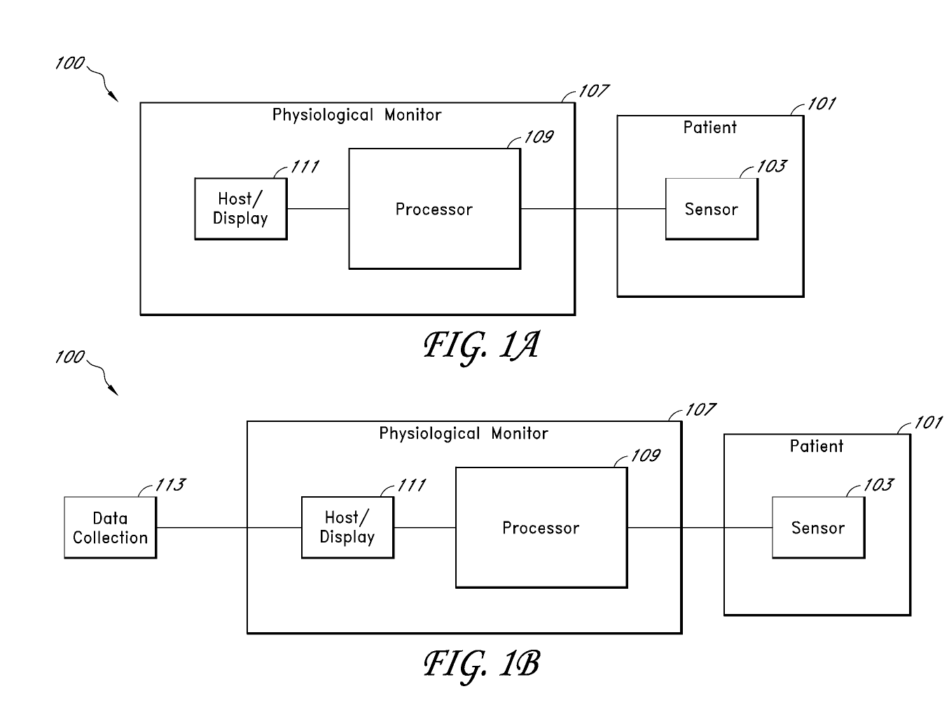

Turning to the Figures, FIG. 1A illustrates an embodiment of a physiological monitoring system. A medical patient 101 is monitored using one or more sensors 103, each of which transmits a signal over a cable 105 or other communication medium to a physiological monitor 107. The physiological monitor 107 includes a processor 109 and, optionally, a host computer or display 111 (“host 111”). The one or more sensors 103 include sensing elements, such as acoustic piezoelectric devices, electrical ECG leads, or the like. Each sensor 103 generates a signal by measuring a physiological parameter of the patient 101. The signal is then processed by one or more processors 109. The one or more processors 109 then communicate the processed signal to the host 111. In an embodiment, the host 111 is incorporated in the physiological monitor 107. In another embodiment, the host 111 is a separate computer or display from the physiological monitor 107.

FIG. 1B illustrates another embodiment of a physiological monitoring system. In this embodiment, the monitoring system also includes a data collection device 113 that receives as an input the output from the host 111 or, alternatively, an output directly from the processor 109. The data output from the host 111 or the processor 109 is transferred to the data collection device 113 over a cable 115. In an embodiment, the data collection device 113 is a personal computer, server, memory unit, or other electronic storage device having a storage capacity suitable for storing the data output from the physiological monitor 107.

For clarity, a single block is used to illustrate the one or more sensors 103 shown in FIGS. 1A and 1B. It should be understood that the sensor 103 block shown in the Figures is intended to represent one or more sensors. In an embodiment, the one or more sensors 103 include a single sensor of one of the types described below. In another embodiment, the one or more sensors 103 include at least two acoustic sensors. In still another embodiment, the one or more sensors 103 include at least two acoustic sensors and one or more ECG sensors. In each of the foregoing embodiments, additional sensors of different types are also optionally included. Other combinations of numbers and types of sensors are also suitable for use with the physiological monitoring system 100.

In some embodiments of the systems shown in FIGS. 1A and 1B, all of the hardware used to receive and process signals from the sensors are housed within the same housing. In other embodiments, some of the hardware used to receive and process signals is housed within a separate housing. In addition, the physiological monitor 107 of certain embodiments includes hardware, software, or both hardware and software, whether in one housing or multiple housings, used to receive and process the signals transmitted by the sensors 103.

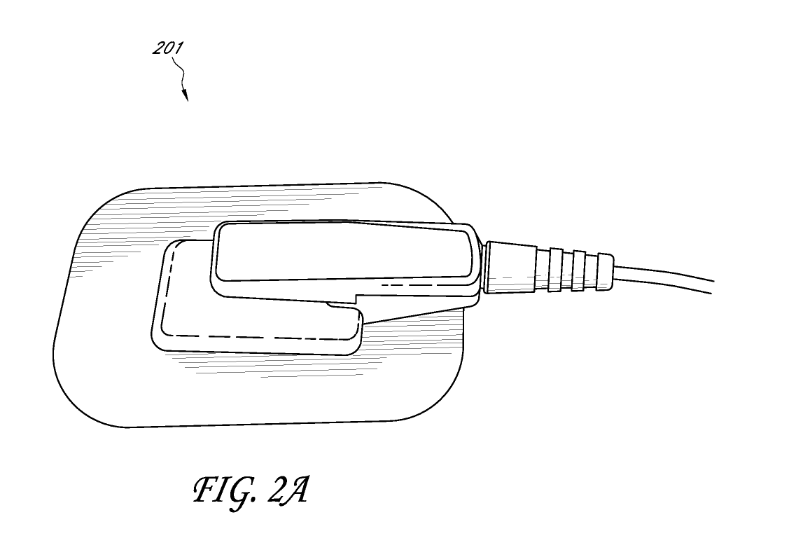

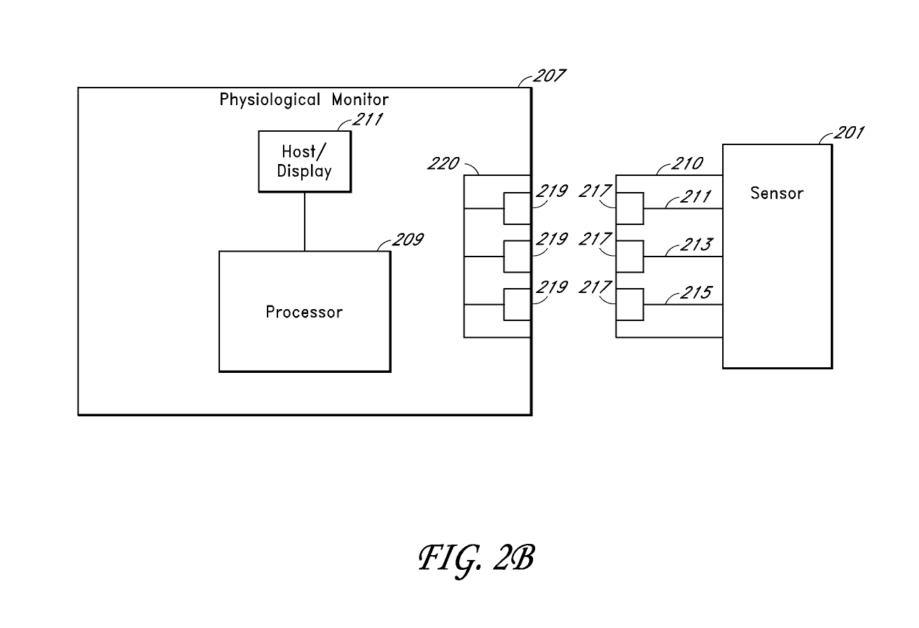

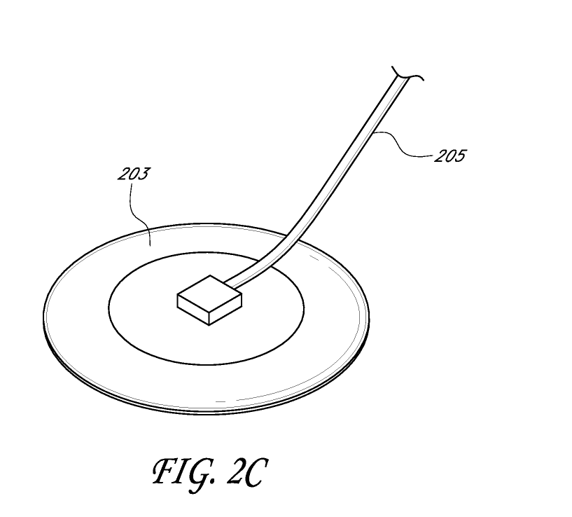

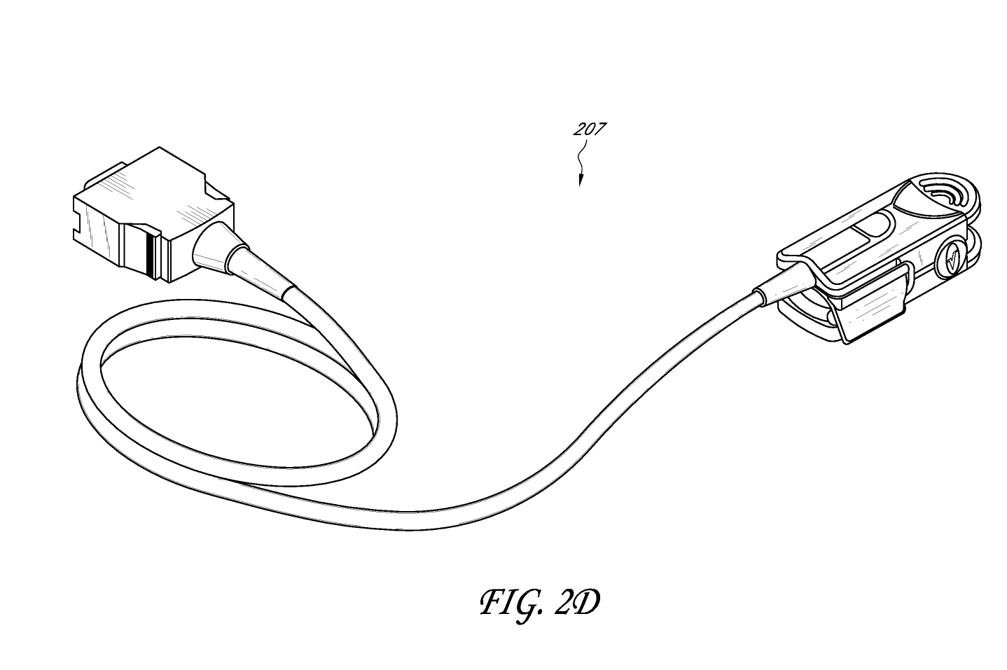

FIG. 2A illustrates an embodiment of an acoustic sensor 201 suitable for use with either of the physiological monitors shown in FIGS. 1A and 1B. In an embodiment, the acoustic sensor 201 includes a sensing element, such as, for example, a piezoelectric device or other acoustic sensing device. The sensing element generates a voltage that is responsive to vibrations generated by the patient, and the sensor includes circuitry to transmit the voltage generated by the sensing element to a processor for processing. In an embodiment, the acoustic sensor 201 includes circuitry for detecting and transmitting information related to biological sounds to a physiological monitor. These biological sounds may include heart, breathing, and/or digestive system sounds, in addition to many other physiological phenomena. The acoustic sensor 201 in certain embodiments is a biological sound sensor, such as the sensors described in U.S. Provisional No. 60/893,858, filed Mar. 8, 2007, entitled “Multi-Parameter Sensor for Physiological Monitoring”. In other embodiments, the acoustic sensor 201 is a biological sound sensor such as those described in U.S. Pat. No. 6,661,161, which is also incorporated by reference herein. Other embodiments include other suitable acoustic sensors known to those of skill in the art.

As shown in FIG. 2B, in an embodiment, the acoustic sensor 201 includes a cable 210 or lead. The cable 210 typically carries three conductors within a shielding: one conductor 211 to provide power to a physiological monitor 207, one conductor 213 to provide a ground signal to the physiological monitor 207, and one conductor 215 to transmit signals from the sensor 101 to the physiological monitor 207. In some embodiments, the “ground signal” is an earth ground, but in other embodiments, the “ground signal” is a patient ground, sometimes referred to as a patient reference, a patient reference signal, a return, or a patient return. In some embodiments, the cable 210 carries two conductors within a shielding, and the shielding layer acts as the ground conductor. Electrical interfaces 217 in the cable 210 enable the cable to electrically connect to electrical interfaces 219 in a connector 220 of the physiological monitor 207. In another embodiment, the sensor 201 and the physiological monitor 207 communicate wirelessly. Additional information relating to the acoustic sensor 201, including other embodiments of the sensor 201 and its interface with the physiological monitor 207, is included in U.S. Provisional No. 60/893,858, filed Mar. 8, 2007, entitled “Multi-Parameter Sensor for Physiological Monitoring”.

FIG. 2C illustrates an embodiment of an ECG sensor 203 suitable for use with either of the physiological monitors shown in FIGS. 1A through 2B above. The ECG sensor includes an electrode adapted to be connected to the body of a patient and to measure electrical impulses generated by the patient's heart. A cable 205 couples the electrode with a physiological monitor.

FIG. 2D illustrates an embodiment of a pulse oximetry sensor 207, also suitable for use with either of the physiological monitors shown in FIGS. 1A through 2B. The pulse oximetry sensor 207 includes an emitter that generates light at two or more wavelengths that is transmitted through a portion of the body tissue of the patient and which is then collected by a detector contained on the sensor 207. The electrical signal created by the detector is transmitted through a cable to a physiological monitor, where the electrical signal is used to determine the oxygen saturation of hemoglobin contained in the patient's blood.

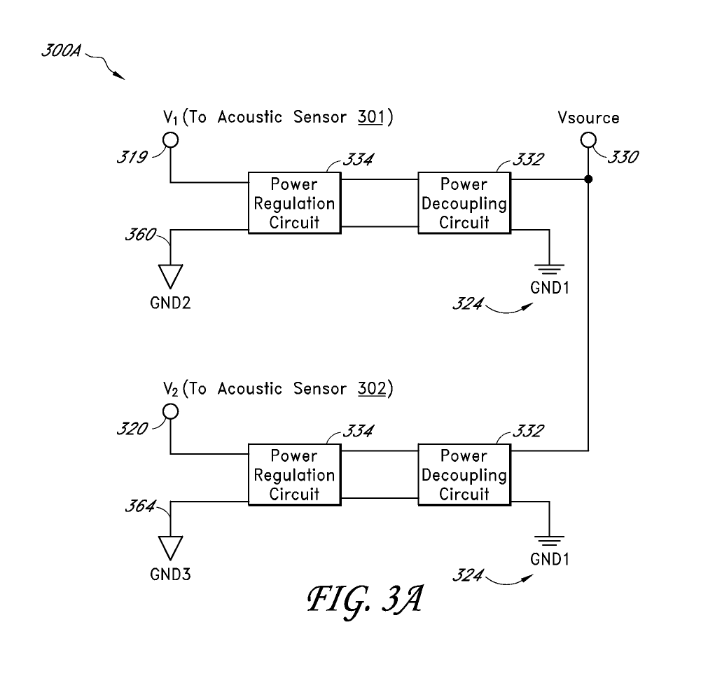

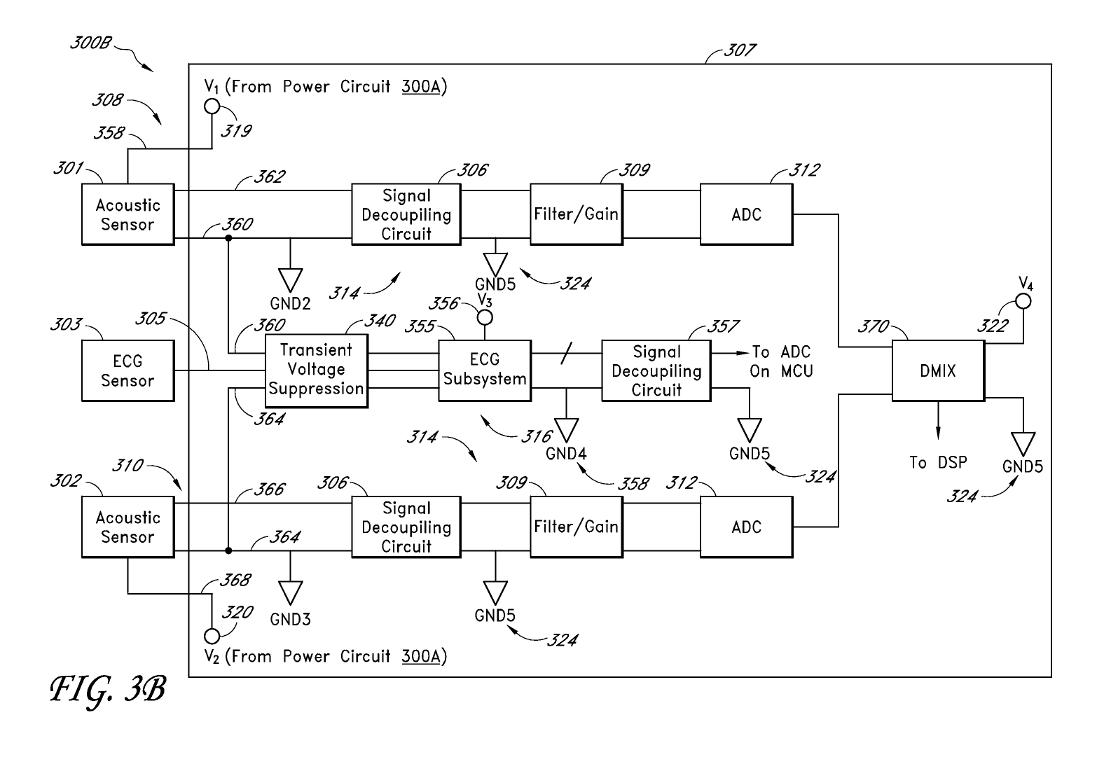

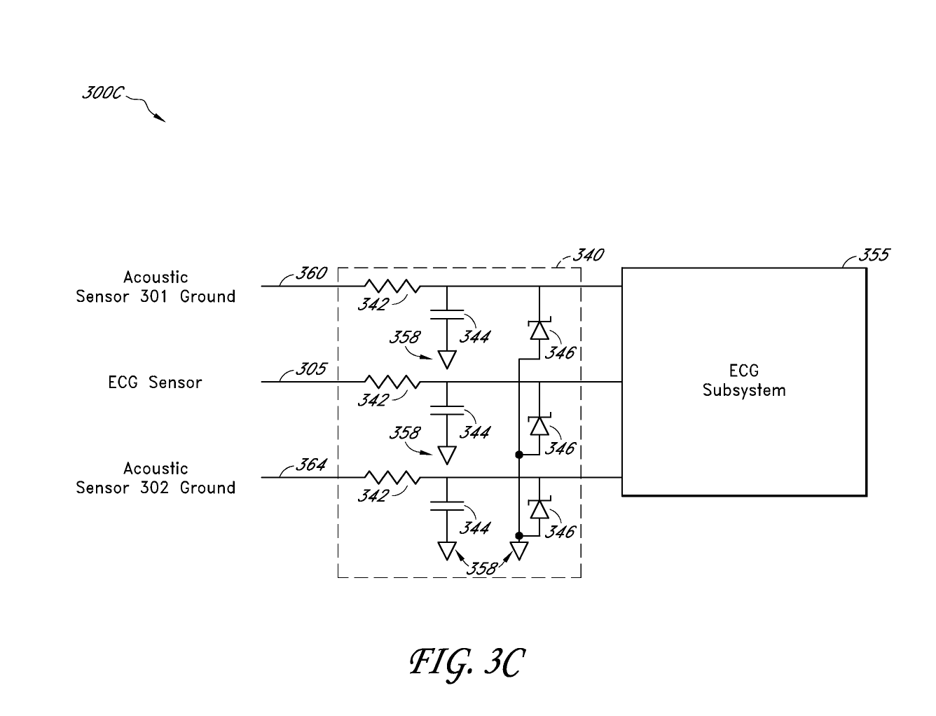

FIG. 3A illustrates, in schematic block diagram form, an embodiment of a power supply circuit 300A. In the embodiment shown, a voltage source 330 provides voltage from a wall outlet, generator, battery, or other voltage source to acoustic sensors (shown in FIGS. 3B, 3C) through power decoupling circuits 332 and power regulation circuits 334. Advantageously, the power supply circuit 300A of certain embodiments prevents potentially dangerous high voltage signals from reaching a medical patient and sensitive electronic components.

The power decoupling circuit 332 electrically decouples the voltage source 330 from acoustic sensors (shown in FIGS. 3B, 3C) which may be attached to a patient. The power decoupling circuit 332 may be implemented in several ways. In one embodiment, the power decoupling circuit 332 is a DC-DC converter. In other embodiments, the power decoupling circuit 332 may be an optocoupler, a wireless connection, or other suitable decoupling device.

In implementations where the power decoupling circuit 332 is a DC-DC converter, the DC-DC converter typically includes a transformer having two coils of wire wound around a magnetic core material such as iron or steel. The coils are not coupled electrically; that is, they do not make electrical contact. Instead, a switching circuit selectively applies a voltage to one coil of the transformer such that the voltage signal passes between the coils through magnetic fields (e.g., by inductance).

In embodiments where the power decoupling circuit 332 is an optocoupler, the optocoupler includes a light-emitting diode (LED) that transmits an optical signal to a phototransistor. The phototransistor converts the optical signal into a voltage signal. The LED and the phototransistor are not connected electrically but instead communicate optically. Thus, the optocoupler provides the same or similar benefits of electrical decoupling as a DC-DC converter.

In addition, in some implementations the power decoupling circuit 332 may decrease or step down the voltage from the voltage source 330 to a lower voltage. For example, if the power decoupling circuit 332 is a DC-DC converter, the coil windings of the DC-DC converter may be configured to reduce an incoming high voltage (e.g., 120 volts) from the voltage source 330 to a lower voltage appropriate for use by the acoustic sensor, such as 3.3, 5, 9 volts, or another appropriate voltage. By reducing the voltage delivered to the acoustic sensors and therefore to the patient, the power decoupling circuit 332 protects the sensors and the patient.

In the depicted embodiment, two power decoupling circuits 332 are shown. Multiple power decoupling circuits 332 enable the power supply circuit 300A to provide a separate voltage supply 319, 320 and a separate ground line 360, 364 to each acoustic sensor from a single voltage source 330. Because the power decoupling circuits 332 decouple the voltage source 330 from each acoustic sensor, separate voltage supplies 319, 320 (denoted V₁ and V₂ respectively), which may be equal or different in value, are provided to the acoustic sensors. Likewise, separate ground lines 360, 364 are provided to the acoustic sensors.

In one embodiment, by virtue of having separate ground lines 360, 364, the acoustic sensors output a potentially different current signal on each respective ground line 360, 364. In addition, because the ground lines 360, 364 are separate, a voltage potential can exist between the ground lines 360, 364. This voltage potential between the ground lines 360, 364 can be measured to provide an ECG signal, as described further below under FIG. 3B.

The power regulation circuit 334 receives a voltage signal output from the power decoupling circuit 332 and converts the voltage signal, which may be time-varying or rippled, into a stable, DC voltage signal. In one embodiment, the power regulation circuit 334 includes one or more diode rectifiers, one or more smoothing capacitors, and a voltage regulator (not shown). As will be understood by those of ordinary skill in the art, diode rectifiers and capacitors can be combined to convert a time-varying or alternating current (AC) signal into a direct current (DC) signal. In addition, the voltage regulator receives the rectified DC voltage and produces a steady output DC voltage. Thus, the power regulation circuit 334 of certain embodiments provides a well-regulated voltage signal to the acoustic sensors 301, 302.

In alternative embodiments, the power regulation circuit 334 may be transposed with the power decoupling circuit 332, such that the voltage source 330 provides voltage directly to the power regulation circuit 334. In one such embodiment, there is only one power regulation circuit 334, and the power regulation circuit 334 provides a single voltage output to two separate power decoupling circuits 332.

In certain embodiments, the power decoupling circuit 332 includes the functions of the power regulation circuit 334. Thus, in some implementations, a single off-the-shelf integrated circuit may be used to perform the functions of both power decoupling and power regulation. Moreover, in certain embodiments, a single power decoupling circuit 332 with multiple channels is employed instead of two separate power decoupling circuits 332.

FIG. 3B illustrates, in schematic block diagram form, an embodiment of a signal acquisition system 300B. In the embodiment shown, two acoustic sensors 301, 302 are each connected to a physiological monitor 307 by a cable 308, 310 or other suitable communication device. Each acoustic sensor 301, 302 outputs a voltage signal composed of time-varying voltages corresponding to physiological sounds from the patient. The voltage signals are communicated by the cables 308, 310 to the physiological monitor 307. An acoustic signal channel 314 is associated with each acoustic sensor 301, 302. For each acoustic signal channel 314, the signal acquisition system 300B includes a filter/gain adjustment stage 309, an analog-to-digital converter (ADC) 309, and a signal decoupling circuit 306. Each acoustic signal channel 314 is routed to a digital signal mixer (DMIX) 370, which transmits a combined digital signal to a processor, such as a digital signal processor (DSP).

The cables 308, 310 in one embodiment incorporate the same structure and functions of the cable 210 described in FIG. 2B above. The depicted cables 308, 310 each include a power line 358, 368 that receives power from a voltage supply 319, 320, a signal line 362, 366, and a ground line 360, 364. Each voltage supply 319, 320 supplies power to the respective acoustic sensor 301, 302. In one embodiment, power from the voltage supplies 319, 320 is supplied to an information element, a parasitic power supply, and sensing circuitry in the acoustic sensors 301, 302 in a manner described in U.S. Provisional No. 60/893,850, filed Mar. 8, 2007, entitled “Backward Compatible Physiological Sensor with Information Element”.

The voltage supplies 319, 320 in one embodiment are separate voltage supplies provided by the power supply circuit 300A, described above in connection with FIG. 3A. Because the voltage supplies 319, 320 are separate, the ground lines 360, 364 are also separate, such that each ground line 360, 364 is not connected to the other. In other words, each ground line 360, 364 floats relative to the other and a potential voltage difference exists between each ground line 360, 364. Advantageously, the floating ground lines 360, 364 of the acoustic sensors 301, 302 are useful for acquiring an ECG signal from a patient, as described more fully below.

In an embodiment, each signal decoupling circuit 306 receives a signal from the acoustic sensor 301, 302 through the signal line 362, 366 on each channel 314. The signal decoupling circuit 306 can be a DC-DC converter (such as the DC-DC converter described above), an optocoupler (such as also described above), or any other device that electrically decouples a signal. In addition, while one signal decoupling circuit 306 is shown on each channel, multiple signal decoupling circuits 306 may be used on each channel in certain embodiments to decouple a bus of data output from each ADC 312. Alternatively, a single multi-channel signal decoupling circuit 306 may be employed.

Electrical decoupling in certain embodiments creates a high degree of electrical isolation between components. In some implementations, this isolation is complete or nearly complete. However, in other embodiments, electrical decoupling occurs above a certain threshold, such that leakage currents above the threshold are prevented from passing between electrical contacts, e.g., between coils of a DC-DC converter. For example, in one implementation, electrical decoupling prevents leakage currents greater than 5 mA (milliamps) from passing between electrical components. In another example, electrical decoupling prevents leakage currents greater than 0.05 mA from passing between electrical components.

The signal decoupling circuit 306 also prevents potentially hazardous defibrillation currents or electrostatic discharge currents from damaging circuit components to the right of the signal decoupling circuits 306, e.g., the DMIX 370, the DSP, or one or more processors. Moreover, to further decouple these components, different power supplies are used on each side of the signal decoupling circuit 306. For example, a voltage supply 322 (also denoted V₄) and a ground line 324 (also denoted GND5) are connected to the DMIX 370, the DSP, and other components. In one embodiment, the voltage supply 322 is also electrically decoupled from the voltage source 330 through a power decoupling circuit 332 (see FIG. 3A), and hence the ground line 324 is a separate ground line from the ground lines 360 and 364 and the ECG lead 305.

In certain embodiments, the electrical decoupling between different voltage supplies 319, 320, and 322 provided by the signal decoupling circuits 306 facilitates each ground line 360, 364 floating relative to one another. There consequently exists a possible difference in potential (e.g., voltage) between each ground line 360, 364, which may be advantageously used to determine an ECG signal, as will be discussed in further detail below. The signal decoupling circuits 306 in certain embodiments therefore provide both power and signal decoupling to components in the signal acquisition system 300B.

Another advantage provided by the signal decoupling circuits 306 is that components to the right of the signal decoupling circuits 306 (e.g., the filter/gain adjustment stages 309 and ADCs 312) may all share a common ground 324 (denoted GND5). As a result, multiple sensors having different ground lines, e.g., the acoustic sensors 301, 302, ECG sensors, and other sensors, can communicate with one processor.

Though the signal decoupling circuit 306 is shown to the left of the filter/gain adjustment stage 309 and the ADC 312, the signal decoupling circuit 306 could optionally be placed after the filter/gain adjustment stage 309 or after the ADC 312. Other circuit components in the signal acquisition system 300B may likewise be rearranged without loss of functionality. In one embodiment, placing the signal decoupling circuit 306 after the ADC 312 allows the signal decoupling circuit 306 to transmit digital, rather than analog, signals. This arrangement reduces noise in the signal acquisition system 300B because the signal decoupling circuit 306 is less likely to add distortion-inducing noise to a digital signal than to an analog signal.

The filter/gain adjustment stage 309 in certain embodiments includes a filter or plurality of filters that selectively remove portions of the signal or otherwise shape the signal obtained from each acoustic sensor 301, 302. In addition, in an embodiment, the filter/gain adjustment stage 309 includes an adjustable gain stage that amplifies the signal to an appropriate level for analog-to-digital conversion and for later digital signal processing. The adjustable gain stage in certain embodiments operates by automatically adjusting the amplification or gain level of the voltage signal, without intervention by a human operator, in the manner described in U.S. Provisional No. 60/893,856, filed Mar. 8, 2007, entitled “Physiological Monitor With Fast Gain Data Acquisition”. In certain embodiments, the filter/gain adjustment stage 309 separates the signal into two or more separate signals. In such cases, it will be understood that each acoustic signal channel 314 actually includes two or more signal channels (not shown). In addition, it will be understood that the filter/gain adjustment stage 309 may be modified or otherwise removed from the physiological monitoring system 307 in some implementations.

The ADC 312 on each channel 314 receives the amplified signal from the filter/adjustable gain stage 306. In certain embodiments, the ADC 312 is a delta-sigma converter, such as the delta-sigma converter described in U.S. Pat. No. 5,632,272, which is hereby incorporated by reference in its entirety. In certain embodiments, the ADC 312 samples the signal into discrete voltage values and then converts the discrete sampled signal into a digital signal represented by digital values. In other embodiments, sampling and analog-to-digital conversion are performed by separate circuit components, such as a sample-and-hold circuit in combination with the ADC 312. In addition, in alternative embodiments, one multi-channel ADC is used in place of the two ADCs 309.

The output signal from each of the ADCs 309 is routed to the DMIX 370 in one embodiment. The DMIX 370 combines the signals from each channel 314 to provide a single output signal and routes the output signal to a digital signal processor (DSP). The multiple signal inputs to the DMIX 370 are interlaced using time division multiplexing (TDM), thereby providing communication of several signal streams from the acoustic channels 314 into a single communication channel. For example, in an embodiment, each channel 314 includes at least two voltage signals corresponding to two separate gain levels provided by the filter/gain adjustment stages 306. Accordingly, in an embodiment, the DMIX 370 accommodates at least four signal streams, which are interlaced and outputted as a single communication channel that is routed to the DSP.

In one embodiment, the DMIX 370 is able to perform TDM because of overclocking or oversampling by the ADCs 309. Each ADC 312 oversamples the signal received from the filter/gain adjustment stage 309 and thereby creates gaps in the digitized signal where no or little information is contained. In one embodiment, these gaps are regularly spaced apart at the same or approximately the same width by each ADC 312. The DMIX 370 in one embodiment creates a new signal by alternately taking samples from each ADC 312 output signal when the signal from the other ADC 312 is in a blindspot or gap. Thus, the DMIX 370 constructs a signal which has little or no gaps, alternating between output samples from each ADC 312. In other embodiments, the DMIX 370 is adapted to accommodate more or fewer signal streams.

Alternatively, in certain embodiments the signal acquisition system 300B does not include a DMIX 370. Instead both channels 314 provide a separate signal to the DSP or to multiple DSPs. However, including the DMIX 370 in certain embodiments provides a greater degree of synchronization between the channels 314 and reduces the number of pins on a DSP chip used by acoustic signal inputs.

The signal acquisition system 300B embodiment shown in FIG. 3B is adapted to acquire at least two acoustic signals from a patient, and to provide those signals to a digital signal processor, microcontroller, or the like. In an embodiment, the at least two acoustic signals obtained using the signal acquisition system 300B embodiment shown in FIG. 3B are the only signals used by the physiological monitor 307 to determine desired physiological parameters. In other embodiments, additional signals (either acoustic or non-acoustic) are obtained and used by the physiological monitor 307 to determine additional physiological parameters or other measurements.

As one example of an additional signal, an ECG sensor 303 obtains electrical signals from a patient. In one embodiment, the ECG sensor 303 is an electrode such as the ECG sensor 203 of FIG. 2C. The ECG sensor 303 is connected to the physiological monitor 307 by a cable 311 which includes an ECG lead 305. The ECG sensor 303 outputs a current signal composed of time-varying current corresponding to electrical signals produced by the patient's cardiac system. The current signals are communicated from the ECG sensor 303 through the ECG lead 305 to the physiological monitor 307. An ECG signal channel 316 is associated with the ECG sensor 303.

In an embodiment, the ECG lead 305 transmits the current signal obtained from the patient to an ECG subsystem 355. The ECG subsystem of certain embodiments is a circuit which generates composite ECG signals from 2, 3, 5, 12, or any number of leads by measuring voltage differences between the leads. In one embodiment, the ECG subsystem 355 includes an application-specific integrated circuit (ASIC) designed specifically to generate an ECG signal from various inputs. Commercially available ECG ASICs may be used, such as the ECG ASIC Part No. 91163 from Welch Allyn®, the datasheet of which is hereby incorporated by reference in its entirety.

The ECG subsystem 355 receives power from a voltage supply 356 (denoted V3), and the ECG subsystem 355 is connected to a ground line 358 (denoted GND4). In one embodiment, the voltage supply 356 is electrically decoupled from the voltage source 330 through a power decoupling circuit (e.g., the power decoupling circuit 332, shown in FIG. 3A). Thus, the ground line 358 is separate from the ground lines 324, 360, 364, and the ECG lead 305.

In the embodiment shown, the ESC subsystem 355 generates an ECG signal from 3 signal input lines. Signal input lines to the ECG subsystem 355 include the ECG lead 305 from the ECG sensor 303 and the ground lines 360, 364 from each of the two acoustic sensors 301, 302. In an embodiment, the acoustic sensors 301, 302 and the ECG sensor 303 are placed at appropriate locations on a patient in order to obtain time-varying voltage signals suitable for an ECG determination. For example, and without limitation, the acoustic sensor 301 may be attached to the patient on the patient's neck (tracheal lead), the acoustic sensor 302 may be attached to the patient's chest over the patient's heart, and the ECG sensor 303 may be attached to the patient's abdomen, arm, or leg. In one embodiment, the sensors are not aligned with one another.

Each of the ground lines 360, 364 from the acoustic sensors 301, 302 float relative to one another because they are electrically decoupled by the signal decoupling circuits 306, as explained above. In addition, the ground lines 360, 364 float relative to the ECG lead 305 because they are electrically decoupled from one another by a signal decoupling circuit 357, which is described in detail below. In addition, in one embodiment, the ground lines 360, 364, and the ECG lead 305 float relative to each other due to the power decoupling circuit 332. Consequently, potential voltage differences exist between each ground line 360, 364 and the ECG lead 305. In one embodiment, the peak-to-peak voltage difference between each lead is approximately 1 mV (millivolt). This voltage value, however, can vary significantly depending on the electrical activity of the patient's heart.

The ECG subsystem 355 in one embodiment measures voltage differences between the ground lines 360, 364, and the ECG lead 305. In one embodiment, the ECG subsystem 355 measures the voltage between the ground line 360 and the ECG lead 305, the voltage between the ground line 364 and the ECG lead 305, and the voltage between the ground line 360 and the ground line 364. Using these voltages, the ECG subsystem 355 develops a three-lead ECG signal. Thus, the acoustic sensors 301, 302 in certain embodiments also operate as ECG sensors. Because the acoustic sensors 301, 302 also act as ECG sensors, fewer sensors are attached to the patient than are used in currently available devices.

In certain embodiments, the number of ECG leads and acoustic sensors used to take ECG measurements can vary. For instance, several ECG leads may be added in one embodiment to the signal acquisition system 300B to produce 5- or 12-lead ECG readings. Alternatively, a combination of added acoustic sensors and ECG leads may be used to produce 5- or 12-lead ECG readings. Moreover, solely acoustic sensors may be used to take 3-, 5-, 9-, 12-, 15-lead, or any other number of lead ECG readings. In one embodiment, the ground signals from two acoustic sensors may even be used to generate a two-lead ECG reading. In addition, when multiple sensors are employed, the ECG subsystem 355 may take voltage measurements between fewer than all of the sensors in some implementations.

Because the ECG subsystem 355 measures the voltage differences between the ground lines 360, 364 and the ECG lead 305, in certain embodiments the ground lines 360, 364, and the ECG lead 305 are brought relatively close together. The proximity of the ground lines 360, 364, and the ECG lead 305 creates a risk that high voltage between the ground lines, such as may be created by defibrillator current or electrostatic discharge (ESD), may short the ground lines 360, 364 or the ECG lead 305. In addition, high current from a defibrillator or ESD can damage the ECG subsystem 355 or other components in the signal acquisition system 300B. Accordingly, in certain embodiments, a transient voltage suppression circuit 340 is interposed or otherwise connected between the acoustic sensor ground lines 360, 364, the ECG lead 305, and the ground line 358 of the ECG subsystem 355. The transient voltage suppression circuit 340 might be implemented with, for example, transient voltage suppression diodes, zener diodes, varistors, or the like. In one embodiment, the transient voltage suppression circuit 340 protects against high voltages of up to 3 kV (kilovolts), 5 kV, or higher.

One implementation of a transient voltage suppression circuit 340 is depicted in FIG. 3C. The transient voltage suppression circuit 340 includes resistors 342, shunt capacitors 344, and zener diodes 346. The ground line from each acoustic sensor 301, 302 and the ECG lead 305 from the ECG sensor 303 communicates in series with a resistor 342 and in parallel with a shunt capacitor 344 and a zener diode 346. In the event of a defibrillation current reaching either of the acoustic sensors 301, 302 or the ECG sensor 303, the defibrillation current passes in part through the shunt capacitor 344 to ground 330, minimizing the delivery of such current to the ECG subsystem 355. In addition, the current passes in part through the zener diode 346 to ground 330, further minimizing the delivery of defibrillation current to the ECG subsystem 355. Consequently, sensitive electronic components in the ECG subsystem 355 are protected from harmful currents.

In addition, in certain embodiments, the resistor 342 and shunt capacitor 344 together act as a low-pass filter to protect the ECG subsystem 355 again high frequency signals. In one such embodiment, the resistor 342 and shunt capacitor 344 act as an electrosurgery interference suppression (ESIS) filter by reducing the amount of high frequency voltage sent to the ECG subsystem 355 caused by electrosurgery instruments.

The resistor 342 of various embodiments has a value of 39.2KΩ (kilohms), though several values on the order of kilohms may be chosen (e.g., 10-100 KΩ). The shunt capacitor 344 in certain embodiments has a value of 220 pF (picofarads), though many other values on the order of picofarads may also be chosen (e.g., 100-250 pF). One of skill in the art will appreciate that many other values of the resistors 342 and capacitors 344, other than the ranges described herein, may also be chosen.

Referring to FIG. 3B, the signal output from the ECG subsystem 355 is provided as an input to a signal decoupling circuit 357. While one signal decoupling circuit 357 is shown, in certain embodiments the ECG subsystem 355 outputs multiple signal lines to multiple decoupling circuits 357. Like the signal decoupling circuits 306 described above, the signal decoupling circuit 357 may be implemented as a DC-DC converter, an optocoupler, or the like.

The signal decoupling circuit 357 prevents harmful defibrillation currents and other current spikes from harming delicate electronics such as a processor or microcontroller. In addition, the signal decoupling circuit 357 enables the ECG lead 305 to float with respect to the acoustic sensor ground lines 360, 364, e.g., the ground lines 360, 364 and the ECG lead 305 are not connected. Thus, the signal decoupling circuit 357 facilitates generating a 2-, 3-, or higher lead ECG signal.

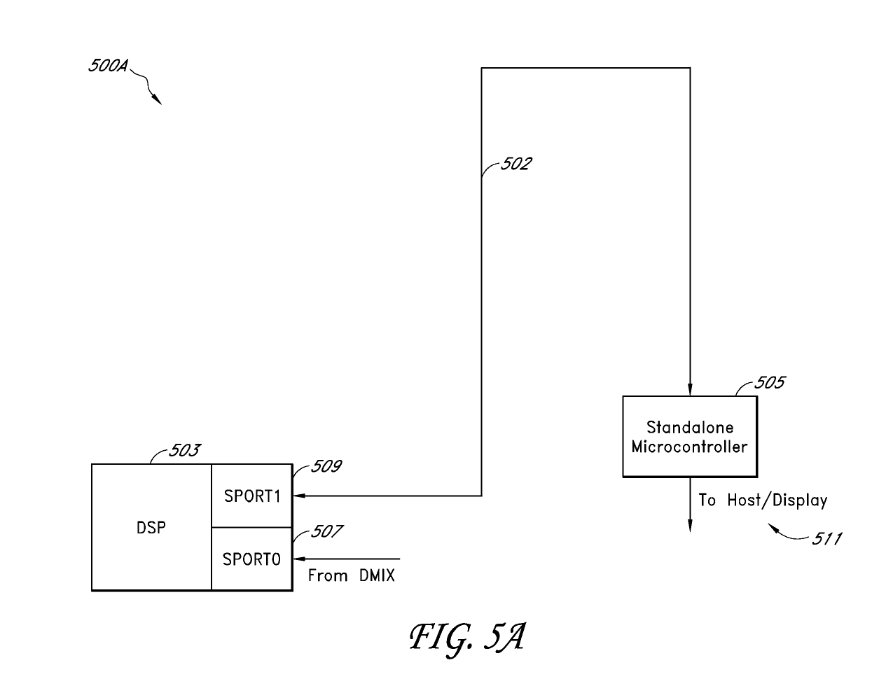

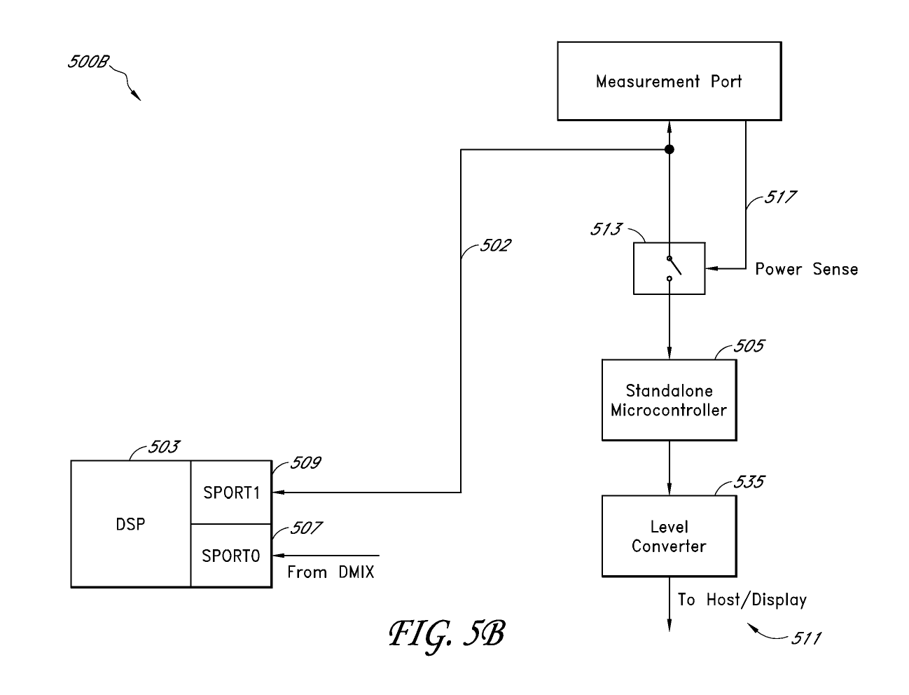

The signal decoupling circuit 357 provides the composite ECG signal to an ADC (see FIGS. 5C and 5D) for analog-to-digital conversion. The ADC then transmits the digital ECG signals to a processor, microcontroller (MCU), or the like, such as is shown in FIGS. 5C and 5D.

While three signal decoupling circuits 306, 357 are shown in FIG. 3B, fewer signal decoupling circuits may be included in alternative embodiments. In addition, if more sensors (e.g., acoustic, ECG, or other forms of sensors) are included in the signal acquisition system 300B, multiple sensors may be combined with one decoupling circuit, or more than one decoupling circuit may be used per sensor. For example, if a 12-lead ECG reading is desired, several acoustic sensors and one or more ECG sensors may be used to determine the 12-lead ECG reading. Of these sensors, several of the sensors may share one or more decoupling circuits. In addition, other sensors, such as capnographic sensors or the like, may be included in the same system but may or may not be coupled with the decoupling circuits.

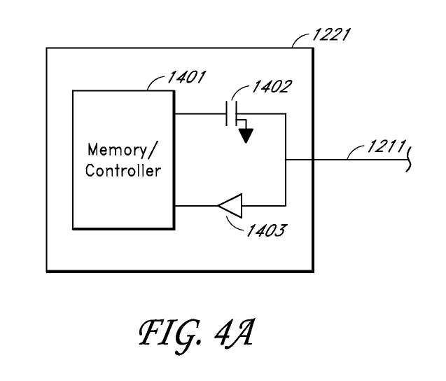

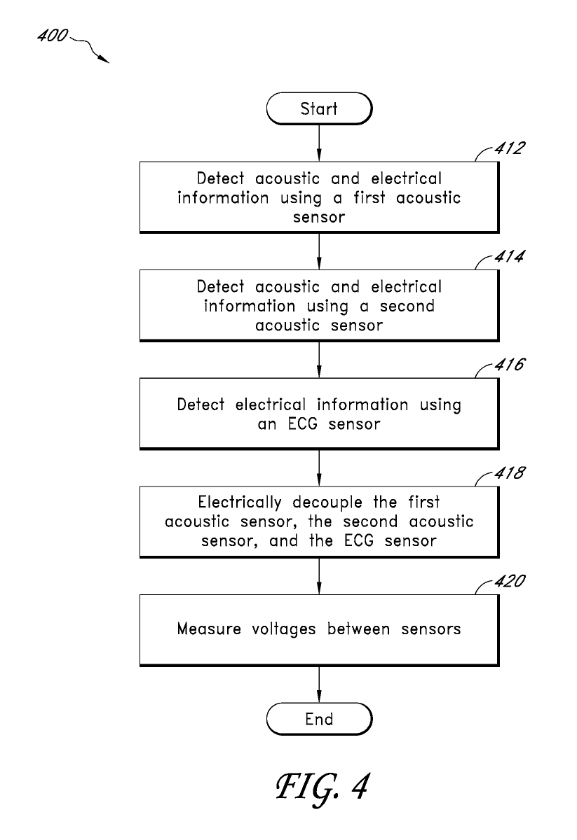

FIG. 4 illustrates certain embodiments of a method of generating an ECG signal. The method 400 may be performed by any of the signal acquisition systems described above. Advantageously, the method 400 provides a process for generating an ECG signal together with an acoustic signal using fewer sensors than are employed in currently available devices.

At 412 the method 400 detects acoustic and electrical information using a first acoustic sensor. At 414 the method 400 also detects acoustic and electrical information using a second acoustic sensor. At 416, the method detects electrical information using an ECG sensor. In certain embodiments, by detecting electrical information from two acoustic sensors and an ECG sensor, the method 400 can generate a 3-lead ECG. In addition, the method 400 generates acoustic information using two of the same sensors employed to generate ECG signals, and hence three sensors are placed on a patient rather than five sensors.

In alternative embodiments, the method 400 detects acoustic and electrical information from only one acoustic sensor and from one ECG sensor. In still other embodiments, the method 400 detects acoustic and electrical information from one acoustic sensor and from two ECG sensors. The method 400 may also detect acoustic and electrical information solely from acoustic sensors, enabling the method 400 to generate ECG signals without using ECG sensors. In some embodiments, the method 400 detects only electrical information even when acoustic sensors are employed. Furthermore, in various embodiments, the method 400 detects acoustic and electrical information using any combination of acoustic and ECG sensors to generate 3-, 5-, 12-lead, 15-lead, or other appropriate number of lead ECG signals.

At 418, the method 400 electrically decouples the first acoustic sensor, the second acoustic sensor, and the ECG sensor. In one embodiment, electrical decoupling at 418 facilitates obtaining different electrical signals from the sensors. The electrical decoupling at 418 may be performed by power decouplers, signal decouplers, or any combination of power decouplers and signal decouplers. For example, in one embodiment of the signal acquisition system 300B described above, electrical decoupling may occur through the power decoupling circuits 332, the signal decoupling circuits 306 and 357, and through other power decoupling circuits (not shown) which supply power to the ECG subsystem 355 and the DMIX 370. Alternatively, the method 400 electrically decouples the sensors using fewer power and/or signal decoupling circuits.

At 420, the method 400 measures voltages between the sensors. In embodiments where the method 400 determines a 3-lead ECG, the sensors may be placed in various locations (not shown), such as on the left arm (LA), right arm (RA), and left leg (LL, or alternatively, right leg (RL)). The method 400 measures the voltages between the sensors at 420, which may be represented by the voltage difference of the sensor on the left arm and the sensor on the right arm (LA−RA), the voltage difference of the sensor on the left leg and the sensor on the right leg (LL−RA), and the sensor on the left leg and the sensor on the left arm (LL−LA). Other locations for the sensors may be chosen without limitation.

In one embodiment, the method 400 also produces waveforms corresponding to the voltage differences viewed over time. These waveforms may include limb leads I, II, and III, where each limb lead captures an electrical view of the heart from a different angle (or “vector”). Lead I corresponds to the voltage difference LA−RA over time, lead II corresponds to the voltage difference LL−RA over time, and lead III corresponds to the voltage difference LL−LA over time. The method 400 may further derive waveforms from augmented limb leads, which also view the heart from a different angle (or vector). For example, the method 400 may derive augmented limb leads aV_{R} (augmented vector right), aV_{L} (augmented vector left), and aV_{F} (augmented vector foot) by calculating various formulas. For instance, in one embodiment, the method 400 determines aV_{R} by the formula RA−(LA+LL)/2. The method 400 determines aV_{L} through the formula LA−(RA+LL)/2, and aV_{F} through the formula LL−(RA+LA)/2.

In addition, if the method 400 is used to compute a higher-lead ECG, such as a 12-lead ECG, the method 400 also computes waveforms from one or more precordial leads V₁, V₂, V₃, V₄, V₅, and V₆, which are placed directly over the chest. In one embodiment, the waveform for each lead Vₙ, where n is any number from 1 to 6, are determined using the formula Vₙ−(RA+LA+LL)/3.

Alternatively, the method 400 at 420 may derive limb leads from other leads, such as limb lead I from the formulas (lead II−lead III) or ((LL−RA)−(LL−LA)). The method 400 may also determine limb lead II from the formulas (lead I+lead III) or ((LL−RA)+(LL−LA)). In addition, the method 400 may determine aV_{R} with the formulas (−(I+III/2)) or (III/2−II), aV_{L} with the formulas ((I−III)/2) or (II/2−III), and aV_{F} with the formulas ((II+III)/2) or (I/2+III).

Hence, it will be appreciated that the method 400 may generate ECG signals by measuring the voltages between sensors at 420 and also by deriving or calculating other voltages from the measured voltages. In one embodiment, the method 400 performs the calculations in software or firmware on the ECG subsystem 355; alternatively, the method 400 may perform the calculations in a separate component, such as a processor.

Turning next to FIGS. 5A through 5D, the figures illustrate several embodiments of a signal processing and routing system 500. Each of the embodiments of the signal processing and routing system 500 is operably associated with one or more of the signal acquisition systems 300B, 300B described above in relation to FIGS. 3A and 3B. In certain embodiments, the signal processing and routing system 500 shown and described includes a multi-function circuit board which can process signals from multiple physiological sensors. Accordingly, the signal processing and routing system 500 of various embodiments eliminates or reduces the need for multiple devices to have unique processing systems, thereby increasing compatibility among such devices.

FIG. 5A illustrates an embodiment of the signal processing and routing system 500A, which includes a digital signal processor (DSP) 503 that is coupled via a communication path 502 to a standalone microcontroller 505. The DSP 503 includes a pair of communication path interfaces, such as implemented by two synchronous serial ports (SPORTs) identified in FIG. 5A as SPORT0 507 and SPORT1 509. The digital signal output from the DMIX 370 (see FIGS. 3A and 3B) is provided as an input to the DSP 503 via the first communication interface SPORT0 507. After processing, the signal output from the DSP 503 is provided as an input to the standalone microcontroller 505 by way of the second DSP communication interface (SPORT1 509) and the communication path 502. The standalone microcontroller 505 then provides an output that is provided to a suitable host or display unit 511 that displays the physiological measurement output to the user.

In an embodiment, the DSP 503 is a processing device based on the Super Harvard Architecture (“SHARC”), such as those commercially available from Analog Devices, Inc. However, the DSP 503 can comprise a wide variety of data and/or signal processors capable of executing programs for determining physiological parameters from input data. In particular, the DSP 503 includes program instructions capable of receiving multiple channels of data related to one or more time-varying voltage signals, such as those provided by the acoustic sensors 201 described herein. In alternative embodiments, however, a processor, microcontroller, or the like performs DSP functions in place of the dedicated DSP 503.

In an embodiment, the standalone microcontroller 505 operates as an instrument manager for a physiological monitor. For example, the microcontroller 505 controls system management, including communications of calculated parameter data and the like to the host or display 511. The microcontroller 505 may also act as a watchdog circuit by, for example, monitoring the activity of the DSP 503 and resetting it when appropriate.

In an embodiment, the host or display 511 communicates with the standalone microcontroller 505 to receive signals indicative of the physiological parameter information calculated by the DSP 503. The host or display 511 includes one or more display devices capable of displaying indicia representative of the calculated physiological parameters measured from the patient. In an embodiment, the host or display 511 may advantageously comprise a handheld housing capable of displaying one or more physiological parameters such as respiratory parameters, cardiac parameters, circulatory parameters, blood analyte concentrations, or other measurable parameters. The host or display 511 may also be capable of storing or displaying historical or trending data related to one or more of the measured parameter values (or contextual data), combinations of the parameters values, other data, or the like. The host or display 511 may also include an audible indicator and a user input device, such as, for example, a keypad, touch screen, pointing device, voice recognition device, or the like. In one embodiment, the host or display 511 is the host described in U.S. patent application Ser. No. 11/367,033, filed on Mar. 1, 2006, titled “Noninvasive Multi-Parameter Patient Monitor,” which is assigned to Masimo Corporation and is incorporated by reference herein.

In an embodiment, the signal processing and routing system 500A is adapted to receive digital signals provided by the signal acquisition circuit described above in relation to FIG. 3A. The signals are provided by the pair of acoustic sensors 201 which, in turn, are attached to a patient 101 in a manner so as to detect biological sounds susceptible to acoustic monitoring. As described above, the acquired signal is converted to a digital signal by the ADCs 309, and is provided by the DMIX 370 as an input to the DSP 503. The DSP 503 processes the digital signal by implementing program code. The DSP 503 in some embodiments uses the digital signal to determine or calculate a value of a physiological parameter of the patient. The DSP 503 might also use the digital signal to calculate respiratory rate or heart rate according to an algorithm. Examples of such algorithms are described in International Application No. PCT/CA2005/000568, published as International Publication No. WO 2005/099562, and International Application No. PCT/CA2005/000536, published as International Publication No. WO 2005/096931, which are hereby incorporated by reference.

In operation, the signal processing and routing system 500A embodiment shown in FIG. 5A supports a standalone mode of operation adapted for determining and displaying physiological parameters in a standalone device. This standalone mode supports monitoring and displaying such patient data to support patient therapy, patient wellness monitoring, patient physiological trend monitoring, clinical research, or other desired purposes. In an embodiment, the DSP 503 receives data signals from the signal acquisition system 300 and uses those signals to determine various physiological parameters of the patient, as described above. The output signals from the DSP 503 are provided as input to the standalone microcontroller 505 via the communication path 502. In an embodiment, the standalone microcontroller 521 provides the signal as an input to a host or display 511, where the physiological parameters are displayed for the user.

Turning to FIG. 5B, in another embodiment, the signal processing and routing system 500B includes a digital signal processor (DSP) 503 that is coupled via a communication path 502 to a standalone microcontroller 505. A switch 513, described more fully below, is included on the communication path 502. The DSP 503 includes a pair of communication path interfaces, such as implemented by two synchronous serial ports (SPORTs) identified in FIG. 5B as SPORT0 507 and SPORT1 509 on the DSP 503. The digital signal output from the DMIX 370 (see FIGS. 3A and 3B) is provided as an input to the DSP 503 via the first communication interface SPORT0 507. After processing, the signal output from the DSP 503 is optionally provided as an input to the standalone microcontroller 505 by way of the second DSP communication interface (SPORT1 509) and the communication path 502. The standalone microcontroller 505 then provides an output that is provided to a suitable host or display unit 511 that displays the physiological measurement output to the user. The signal output from the DSP 503 may instead be provided as an input to a measurement port 515.

In an embodiment, the DSP 503, the standalone microcontroller 505, and the host or display 511 are the same as described above in relation to FIG. 5A. In an embodiment, the measurement port 515 comprises processing circuitry arranged on one or more printed circuit boards capable of installation into the physiological monitor 107, or capable of being distributed as some or all of one or more original equipment manufacture (OEM) components for a wide variety of host instruments monitoring a wide variety of patient information. In an embodiment, the measurement port 515 comprises a printed circuit board that determines and outputs one or more physiological parameters such as pulse rate, plethysmograph data, perfusion quality such as perfusion quality index, signal or measurement quality, and values of blood constituents in body tissue, including for example, SpO2, carboxyhemoglobin (HbCO), and methemoglobin (HbMet). Such printed circuit boards are commercially available from Masimo Corporation. In an embodiment, the measurement port 515 comprises drivers, a front-end, a digital signal processor (DSP), one or more sensor ports, and an instrument manager. The drivers convert digital control signals into analog drive signals capable of driving emitters associated with, for example, a pulse oximetry sensor. The front-end converts composite analog intensity signal(s) from light sensitive detector(s) into digital data input to the DSP contained within the measurement port 515.

In an embodiment, the switch 513 included in the communication path 502 is intended to provide the signal processing and routing system 500B with the capability of operating in at least two modes. In a first mode, corresponding to a closed position of the switch 513, the signal output from the DSP 503 is routed over the communication path 502 through the switch 513 to the standalone microcontroller 505, where the signal is output to the host or display 511. This first mode may correspond, for example, with a standalone mode for the signal processing and routing system 500B. The standalone mode does not include the use of the measurement port 515. The measurement port 515 may be present in the signal processing and routing system 500B but may be nonoperative or not active.

In a second mode, corresponding to an open position of the switch 513, the signal output from the DSP 503 is routed over the communication path 502 and is provided as an input to the measurement port 515. This second mode may be, for example, a daughter board mode for the signal processing and routing system 500B. In this mode, all or portions or the signal processing and routing system 500B are on a daughter board to the measurement port 515, which resides on a motherboard. Also in this mode, the DSP 503 provides a signal that is used as an input to the measurement port 515. The signal from the DSP 503 is then optionally displayed by a display or host (not shown) associated with the measurement port 515, which may be combined with and/or displayed with any other signals acquired by the measurement port 515.

Advantageously, in an embodiment, the switch 513 is controlled by a power sense communication path 517 that detects whether power is supplied to the measurement port 515. For example, in an embodiment, an electrical trace is provided between the switch 513 and the power interface to the measurement port 515. A small resistor is placed in the trace line to limit the voltage applied to the switch 513. The power signal is sent as a binary input to the switch 513. When power is detected as being supplied to the measurement port 515, the switch 513 is opened in order to route the signal output from the DSP 503 to the measurement port 515. This corresponds to the second mode, or daughter board mode, described above. When power is not detected as being supplied to the measurement port 515, the switch 513 is closed, routing the signal output from the DSP 503 to the standalone microcontroller 505. This corresponds to the first mode, or standalone mode, described above.

Turning to FIG. 5C, in another embodiment, the signal processing and routing system 500C includes the DSP 503 (including the pair of communication path interfaces, such as implemented by two synchronous serial ports (SPORTs) 507 and 509), the measurement port 515, and the communication path 502. In one embodiment, these are the same components described above in relation to FIGS. 5A and 5B. In an alternative embodiment, the standalone microcontroller 505 and switch 513 are also included in the signal processing and routing system 500C of FIG. 5C but are not operable. The module 501 further comprises a primary microcontroller 521 that is in electrical communication with the DSP 503 via the communication path 502 and which is in electrical communication with the measurement port 515 via another communication path 522.

In an embodiment, the primary microcontroller 521 includes an analog-to-digital converter (ADC) 523. The primary microcontroller ADC 523 receives the voltage signal from the ECG subsystem 355 of FIG. 3B (e.g., through the signal decoupling circuit 357) and also optionally receives data signals from additional analog inputs 533 described more fully below. In certain embodiments, the ADC 523 is a delta-sigma converter, such as the delta-sigma converter described in U.S. Pat. No. 5,632,272, which is hereby incorporated by reference. In certain embodiments, the ADC 523 samples the received signal(s) into discrete voltage values and then converts the discrete sampled signal(s) into a digital signal(s) represented by digital values. In other embodiments, sampling and analog-to-digital conversion are performed by separate circuit components. In one embodiment, the ADC 523 contains multiple channels to receive multiple, decoupled analog inputs in addition to the acoustic and ECG inputs described above. Alternatively, multiple inputs may be provided to several ADCs on the primary microcontroller 521.

In an embodiment, the primary microcontroller 521 also includes a plurality of communication path interfaces that support communication between the primary microcontroller 521 and other components of the signal processing and routing system 500C and other components of the physiological monitor 107. For example, in an embodiment, at least two such communication path interfaces are implemented by two universal asynchronous receiver/transmitter ports (UARTs) identified in FIG. 5C as UART0 527 and UART1 525, and at least one other communication path interface is implemented by a serial peripheral interface (SPI) bus identified in FIG. 5C as SPI 529. Other types and forms of communication path interface components are provided in other embodiments, as will be recognized by a person of skill in the art. In an embodiment, the UART1 525 communication path interface supports communication over the communication path 522 by and between the primary microcontroller 521 and the measurement port 515. In an embodiment, the communication path 522 supports communication of a digital signal, such as a plethysmographic wave signal, from the measurement port 515 to the microcontroller 521. The UART0 527 communication path interface supports communication with other system components, as described more fully below in relation to FIG. 5D. The SPI 529 communication path interface supports communication over the communication path 502 by and between (on the one hand) the primary microcontroller 521 and (on the other hand) either the measurement port 515 or the DSP 503.

In an embodiment, the primary microcontroller 521 also includes a communication interface to support communication with a data collection host 533 or other external component. For example, in an embodiment, at least one such communication interface is implemented by a universal serial bus (USB) identified in FIG. 5C as USB 531. Alternatively, a standard RS232 serial interface or other form of interface may be used to communicate with the data collection host 533.

In operation, the signal processing and routing system 500C shown in FIG. 5C supports a mode of operation adapted for collecting data corresponding to the measurements of physiological parameters of the patient 101. This data collection mode supports monitoring and storing such patient data to support patient therapy, patient wellness monitoring, patient physiological trend monitoring, clinical research, or other desired purposes. In an embodiment, the DSP 503 receives data signals from the signal acquisition system 300 and uses those signals to determine various physiological parameters of the patient, as described above. In addition, the measurement port 515 determines various physiological parameters of the patient (either the same as or different from those determined by the DSP 503), as also described above. The output signals from each of the DSP 503 and the measurement port 515 are provided as inputs to the primary microcontroller 521 via the communication path 502 and the communication path interface, namely, the SPI 529. In addition, another digital signal output from the measurement port 515 is provided as an input to the primary microcontroller 521 via the communication path 522 and the communication path interface, namely, the UART1 525. In an embodiment, the primary microcontroller 521 operates as an instrument manager for the physiological monitor 107. For example, the primary microcontroller 521 controls system management, including communications of calculated parameter data and the like to a data collection member 533. The primary microcontroller 521 may also act as a watchdog circuit by, for example, monitoring the activity of the DSP 503 and/or the measurement port 515 and resetting it or them when appropriate.

In an embodiment, the data collection member 533 comprises a data storage device such as a personal computer, a server, a disk storage member, or other suitable device. In an embodiment, the data collection member 533 also includes a display for displaying the physiological parameters determined by the physiological monitor 107.

In FIG. 5D, an embodiment of the signal processing and routing system 500D is shown having all of the components described above in relation to FIGS. 5A, 5B, and 5C. In addition, the signal processing and routing system 500D embodiment shown in FIG. 5D includes another communication path 532 supporting communication between the primary microcontroller 521 via the UART0 527 interface and a level convert member 535, which provides an output to the host or display 511. The level convert member 535 converts the voltage signal received as an output from the primary microcontroller 521 (e.g., typically about 3.3 volts) to a level suitable for supplying to the host or display 511 (e.g., typically about 5 volts).

In an embodiment, the signal processing and routing system 500D includes all of the components and communication paths shown in FIG. 5D and described above. In other embodiments, one or more of the components are absent, made inoperative, or are not utilized in order to operate the module 501 according to one or more of the modes of operation described above in relation to FIGS. 5A, 5B, and 5C. For example, the signal processing and routing system 500D may be provided in multiple design variations depending upon the desired mode of operation. In these embodiments, module components are advantageously not included (or rendered inoperable) within the module 501 when not needed for the desired mode of operation, in order to reduce power consumption and/or to obtain other desired benefits.

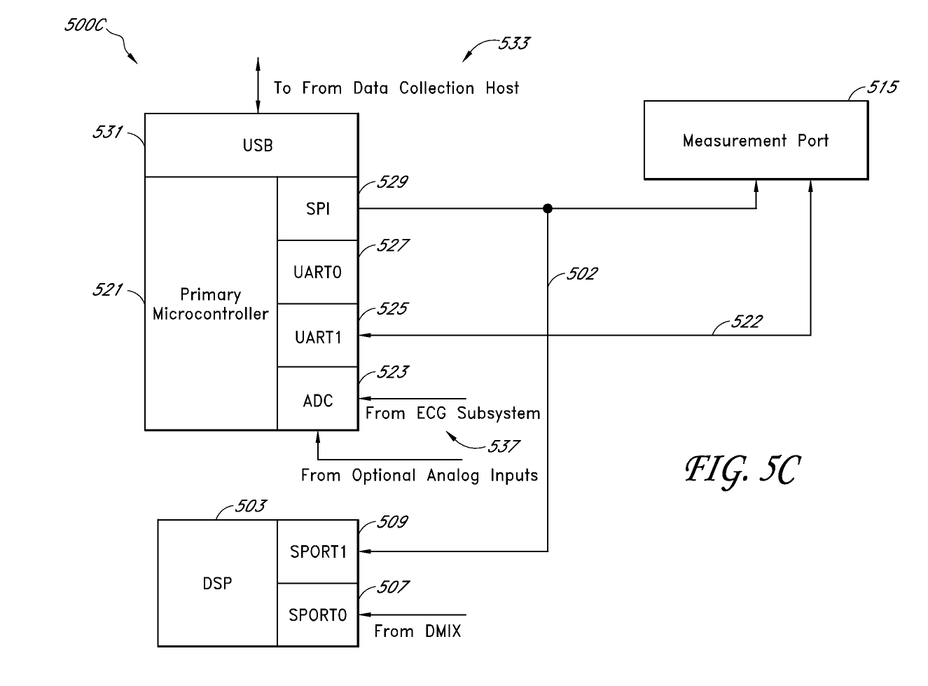

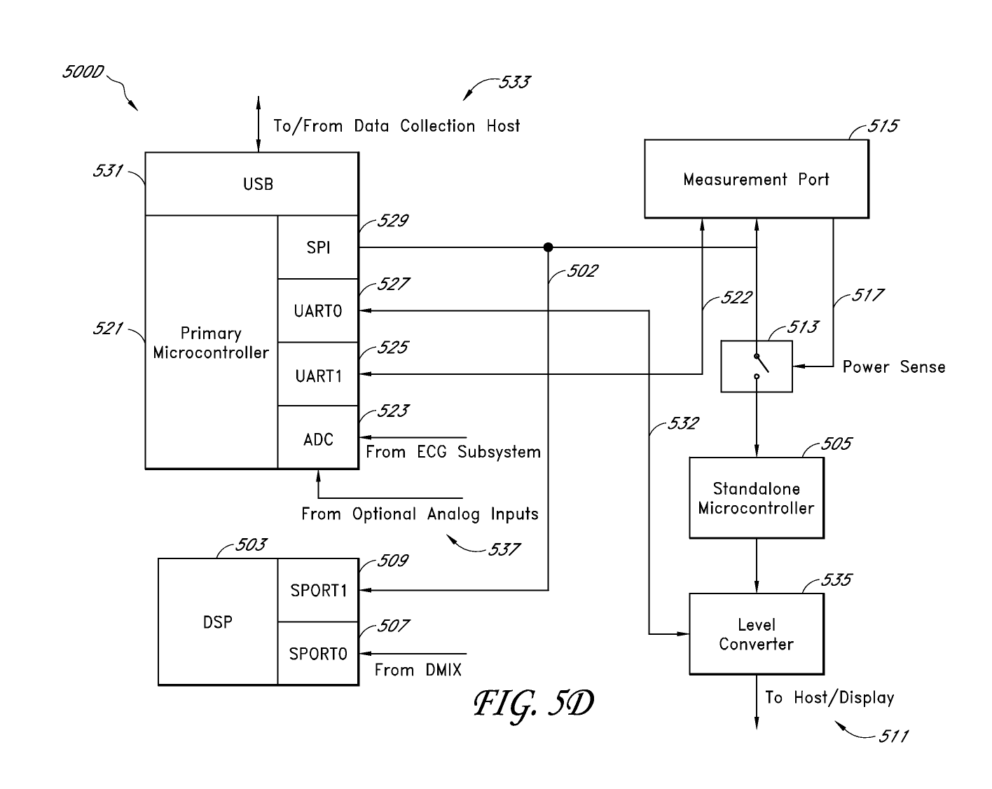

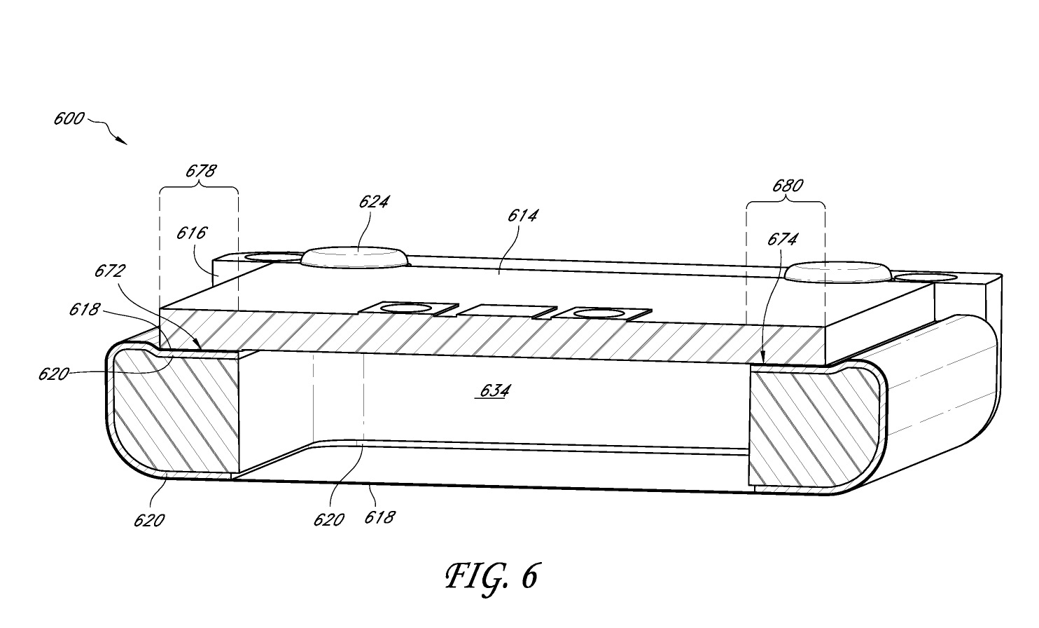

FIG. 6 shows an embodiment of a cross-sectional view of a sensor sub-assembly 600. In one embodiment, the sensor sub-assembly 600 is incorporated in any of the acoustic sensors described above. In addition, the sensor sub-assembly 600 may be incorporated in a multi-parameter sensor described in U.S. Provisional No. 60/893,858, filed Mar. 8, 2007, titled “Multi-Parameter Sensor For Physiological Monitoring,” which is hereby incorporated by reference in its entirety.

A bonding layer 620 is attached to a frame 616 which substantially prevents moisture, such as a patient's sweat, from entering an acoustic chamber or cavity 634 defined by the sensor sub-assembly 600. After a sensing element 618 and bonding layer 620 are attached to the frame 616, a printed circuit board 614 is then provided. The printed circuit board 614 is placed on top of the sensing element 618 such that a first edge 678 of the printed circuit board 614 is placed over a first conductive portion 672 of the sensing element 618, and a second edge 680 of the printed circuit board 614 is placed over the second conductive portion 674 of the sensing element 618.

The printed circuit board 614 is pressed down into the sensing element 618 in the direction of the frame 616. As the printed circuit board 614 is pressed downward, contact bumps 636 (see FIG. 8) of the frame 616 push the bonding layer 620 and sensing element 618 into contact strips located along the first and second sides or edges 678, 680 of the printed circuit board 614. The contact strips of the printed circuit board 614 are made from conductive material, such as gold. Other materials having a good electronegativity matching characteristic to the conductive portions 672, 674 of the sensing element 618, may be used instead. The elasticity or compressibility of the bonding layer 620 acts as a spring, and provides some variability and control in the pressure and force provided between the sensing element 618 and printed circuit board 614.

Once the desired amount of force is applied between the printed circuit board 614 and the frame 616, locking posts 624 are vibrated or ultrasonically welded until the material of the locking posts 624 flows over the printed circuit board 614. The locking posts 624 can be welded using any of a variety of techniques, including heat staking, or placing ultrasonic welding horns in contact with a surface of the locking posts 624, and applying ultrasonic energy. Once welded, the material of the locking posts 624 flows to a mushroom-like shape, hardens, and provides a mechanical restraint against movement of the printed circuit board 614 away from the frame 616 and sensing element 618. By mechanically securing the printed circuit board 614 with respect to the sensing element 618, the various components of the sensor sub-assembly 600 are locked in place and do not move with respect to each other when a multi-parameter sensor incorporating the sensor assembly 600 is placed in clinical use. This prevents the undesirable effect of inducing electrical noise from moving assembly components or inducing unstable electrical contact resistance between the printed circuit board 614 and the sensing element 618.

Therefore, the printed circuit board 614 can be electrically coupled to the sensing element 618 without using additional mechanical devices, such as rivets or crimps, conductive adhesives, such as conductive tapes or glues, like cyanoacrylate, or others. In addition, the mechanical weld of the locking posts 624 helps assure a stable contact resistance between the printed circuit board 614 and the sensing element 618.

The contact resistance between the sensing element 618 and printed circuit board 614 can be measured and tested by accessing test pads on the printed circuit board 614. For example, in one embodiment, the printed circuit board 614 includes three discontinuous, aligned test pads that overlap two contact portions between the printed circuit board 614 and sensing element 618. A drive current is applied, and the voltage drop across the test pads is measured. For example, in one embodiment, a drive current of about 600 mA is provided. By measuring the voltage drop across the test pads the contact resistance can be determined by using Ohm's law, namely, voltage drop (V) is equal to the current (I) through a resistor multiplied by the magnitude of the resistance (R), or V=IR.

The printed circuit board 614 includes various electronic components mounted to either or both faces of the printed circuit board 614. When the multi-parameter sensor assembly 600 is assembled, the electronic components of the printed circuit board 614 may extend into the assembly's cavity 634 or acoustic chamber. To reduce space requirements and to prevent the electronic components from adversely affecting operation of a multi-parameter sensor incorporating the sensor sub-assembly 600, the electronic components can be low-profile, surface mounted devices. The electronic components are often connected to the printed circuit board 614 using conventional soldering techniques, for example the flip-chip soldering technique. Flip-chip soldering uses small solder bumps of predictable depth to control the profile of the soldered electronic components.

In some embodiments, the electronic components include filters, amplifiers, etc. for pre-processing or processing a low amplitude electric signal received from the sensing element 618, prior to transmission through a cable to a physiological monitor. In other embodiments, the electronic components include a processor or pre-processor to process electrical signals. Such electronic components may include, for example, analog-to-digital converters for converting the electric signal to a digital signal and a central processing unit for analyzing the resulting digital signal.

In one embodiment, the printed circuit board 614 also includes a wireless transmitter, thereby eliminating mechanical connectors and cables. For example, optical transmission via at least one optic fiber or radio frequency (RF) transmission is implemented in other embodiments. In other embodiments, the sensor assembly 600 includes a security device, such as an information element, to assure compatibility between the sensor sub-assembly 600 and the physiological monitor to which it is attached. In addition, the sensor sub-assembly 600 can include any of a variety of information storage devices, such as readable and/or writable memories. Information storage devices can be used to keep track of device usage, manufacturing information, duration of sensor usage, other sensor, physiological monitor, and/or patient statistics, etc.

In other embodiments, the printed circuit board 614 includes a frequency modulation circuit having an inductor, capacitor and oscillator, such as that disclosed in U.S. Pat. No. 6,661,161, which is incorporated by reference herein in its entirety. In another embodiment, the printed circuit board 614 includes a field-effect transistor (FET) and a DC-DC converter or isolation transformer and phototransistor. Diodes and capacitors may also be provided. In addition, certain of the circuit components described above under FIGS. 3A, 3B, and 3C may also be provided. In yet another embodiment, the printed circuit board 614 includes a pulse-width modulation circuit.

In yet another embodiment, the printed circuit board 614 includes an information element that communicates calibration and/or identification information to a physiological monitor. For example, in one embodiment, the information element identifies the manufacturer, lot number, expiration date, and/or other manufacturing information. In another embodiment, the information element includes calibration information regarding a multi-parameter sensor incorporating the sensor sub-assembly 600.

In one embodiment, the information element includes an EPROM, EEPROM, ROM, Flash, or other readable memory device. Information from the information element is provided to the physiological monitor according to any communication protocol known to those of skill in the art. For example, in one embodiment, information is communicated according to an I2C protocol. U.S. Provisional No. 60/893,850, filed Mar. 8, 2007, titled “Backward Compatible Physiological Sensor with Information Element,” which is incorporated by reference herein, teaches various methods of communicating information from an information element in a multi-parameter sensor incorporating the sensor sub-assembly 600 to a physiological monitor.

The information element may be provided on or in electrical communication with the printed circuit board 614. In one embodiment, the information element is provided on a cable connected to the printed circuit board.

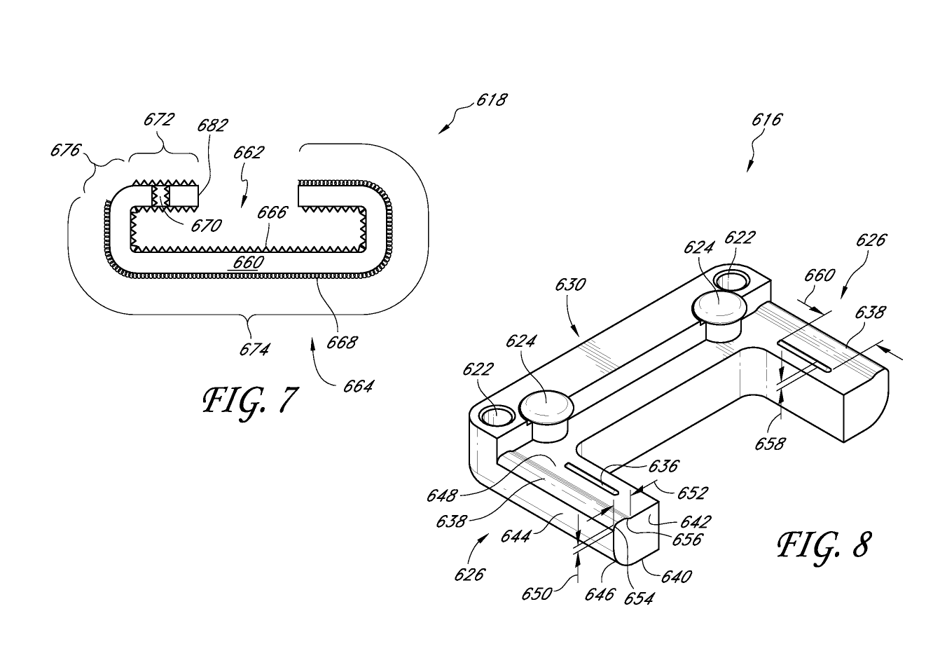

One embodiment of a piezoelectric sensing element 618 is provided in FIG. 7. The sensing element 618 includes a substrate 660 and coatings 662, 664 on each of its two planar faces 666, 668. The planar faces 666, 668 are parallel or substantially parallel to each other along at least a portion of the substrate 660. At least one through hole 670 or via extends between the two planar faces 666, 668. In one embodiment, the sensing element 618 includes two, three, or more through holes 670.

In one embodiment, a first coating 662 is applied to the first planar face 666, the substrate 660 wall of the through holes 670, and a first conductive portion 672 of the second planar face 668. By applying a first coating 662 to the through holes 670, a conductive path is created between the first planar face 666 and the first conductive portion 672 of the sensing element 618. A second coating 664 is applied to a second conductive portion 674 of the second planar face 668. The first conductive portion 672 and second conductive portion 674 are separated by a gap 676 such that the first conductive portion 672 and second conductive portion 674 are not in contact with each other. In one embodiment, the first conductive portion 672 and second conductive portion 674 are electrically isolated or substantially electrically isolated from one another.

In some embodiments, the first and second conductive portions 672, 674 are sometimes referred to as masked portions, or coated portions. The conductive portions 672, 674, can be either the portions exposed to, or blocked from, material deposited through a masking or deposition process. However, in some embodiments, masks are not used. Either screen printing or silk screening process techniques can be used to create the first and second conductive portions 672, 674.

In another embodiment, the first coating 662 is applied to the first planar face 666, an edge portion 682 of the substrate 660, and a first conductive portion 672. By applying the first coating 662 to an edge portion 682 of the substrate 660, through holes 670 can optionally be omitted.

In one embodiment, the first coating 662 and second coating 664 are conductive materials. For example, the coatings 662, 664 can include silver, such as from a silver deposition process. By using a conductive material as a coating 662, 664, the multi-parameter sensor assembly 600 can function as an electrode as well.

Electrodes are devices well known to those of skill in the art for sensing or detecting electrical activity, such as the electrical activity of the heart. Changes in heart tissue polarization result in changing voltages across the heart muscle. The changing voltages create an electric field, which induces a corresponding voltage change in an electrode positioned within the electric field. Electrodes are typically used with echo-cardiogram (EKG or ECG) machines, which provide a graphical image of the electrical activity of the heart based upon signal received from electrodes affixed to a patient's skin.

Therefore, in one embodiment, the voltage difference across the first planar face 666 and second planar face 668 of the sensing element 618 can indicate a piezoelectric response of the sensing element 618, such as to physical aberration and strain induced onto the sensing element 618 from acoustic energy released from within the body. In addition, current through one of the planar faces 666, 668 can indicate an electrical response, such as to the electrical activity of the heart. Circuitry within the multi-parameter sensor assembly 600 and/or within a physiological monitor (not shown) coupled to a multi-parameter sensor incorporating the sensor sub-assembly 600 distinguish and separate the two information streams. One such circuitry system is described above under one or more of FIGS. 1-5.

Referring back to FIG. 7, the sensing element 618 is flexible and can be wrapped at its edges, as shown in FIG. 7. In one embodiment, the sensing element 618 is wrapped around the frame 616, as shown in FIG. 6. In addition, by providing both a first conductive portion 672 and a second conductive portion 674, both the first coating 662 and second coating 664 can be placed into direct electrical contact with the same surface of a printed circuit board 614, as shown in FIG. 6. This provides the advantage of being able to symmetrically place the sensing element 618 under tension, and avoids uneven stress distribution through the sensing element 618.

FIG. 8 shows a cross-sectional view of one embodiment of the frame 616. A patient-contact side 640 of each frame segment 626 extends from an inside surface 642 to an outside surface 644. The patient-contact side 640 transitions to the outside surface 644 via a first curve 646. The dimensions of the first curve 646 are selected such that the sensing element 618 smoothly wraps around the frame 616 when attached. In one embodiment, the first curve 646 has a radius of about 1 mm, or is within the range of about 0.5 to 1.5 mm.

The outside surface 644 transitions to a PCB-contact side 648 via a raised ridge 638. The height 650 and width 652 of the raised ridge 638 are defined by a second curve 654 and a chamfer 656 of the raised ridge 638. In one embodiment, the height 650 is about 0 to 0.70 mm, sometimes about 0.13 mm. In other embodiments, the width 652 is about 0.67 mm, or in the range of about 0 to 1.5 mm. In some embodiments the second curve 654 radius is 0.41 mm, 0 to 1.0 mm. In other embodiments, the chamfer 656 extends at an angle of 30 degrees, or 0 to 90 degrees with respect to the PCB-contact side 648. In the illustrated embodiment, the inside surface 642 is parallel or substantially parallel to the outside surface 644, and the patient-contact side 640 is parallel or substantially parallel to the PCB-contact side 648.

The contact bumps 636 are dimensioned to press a portion of the sensing element 618 into the printed circuit board 614 when the sensor sub-assembly 600 is assembled. In one embodiment, the contact bumps 636 have a height 658 of about 0.26 mm, or in the range of about 0.2 to 0.3 mm. The height 658 is generally selected to provide adequate force and pressure between the sensing element 618 and printed circuit board 614 as is described above.

In one embodiment, the contact bumps 636 have a triangular cross-sectional shape. The triangular cross-sectional shape allows greater pressure between the sensing element 618 and printed circuit board 614. However, in other embodiments, the contact bumps 636 have a trapezoidal, semi-circular, or semi-elliptical cross-sectional shape. The particular cross-sectional shape may be selected to control the pressure and force between the printed circuit board 614 and sensing element 618. By controlling pressure and force, the contact resistance between the two conductive surfaces of the printed circuit board 614 and sensing element 618 can be controlled.

In certain embodiments of the above-described physiological monitoring system, it may be desirable to retrofit an existing system to incorporate, for example, added acoustic monitoring capability. Existing systems, however, may have a limited number of conductors. Therefore, it may be desirable to utilize existing conductors for more than one purpose, e.g. for both power delivery and communication with an information element. In addition, it is often advantageous to reduce the number of conductors used to communicate between a physiological monitor and a sensor. In this regard, certain embodiments, such as those described below, provide the advantage of reducing sensor lead conductors, which in turn reduces design complexity, improves cable flexibility, and simplifies manufacturing requirements.

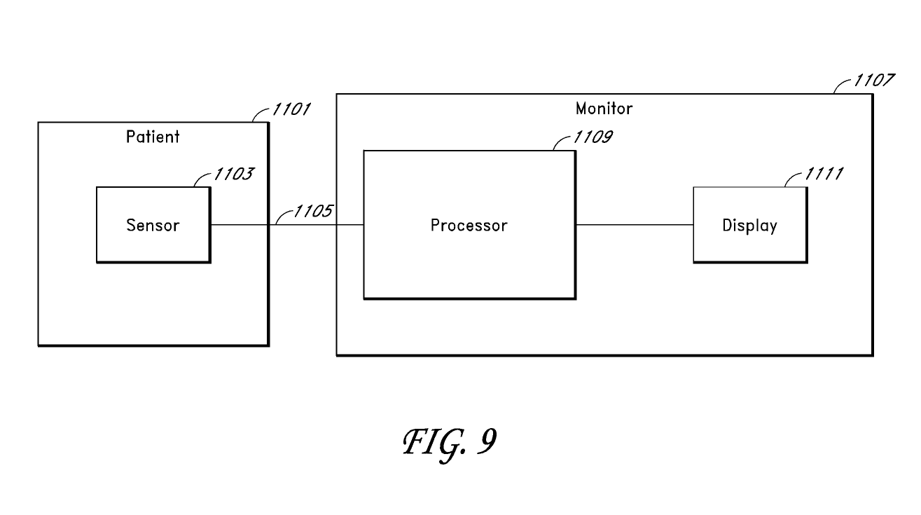

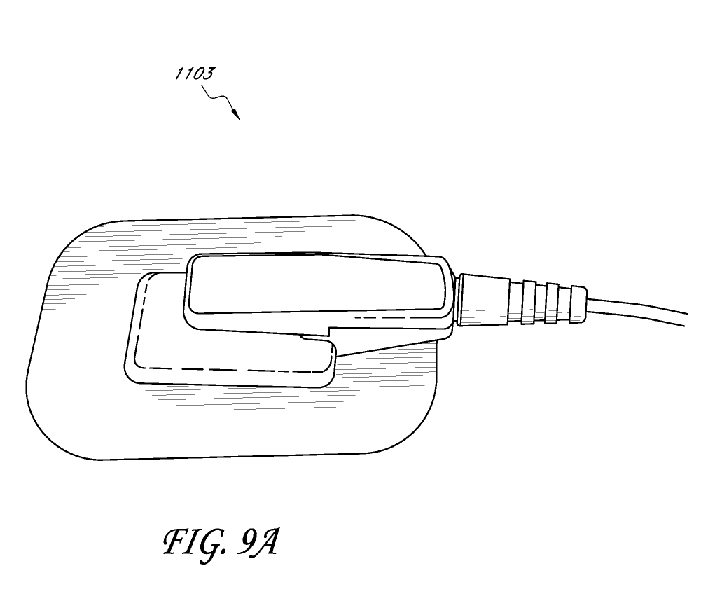

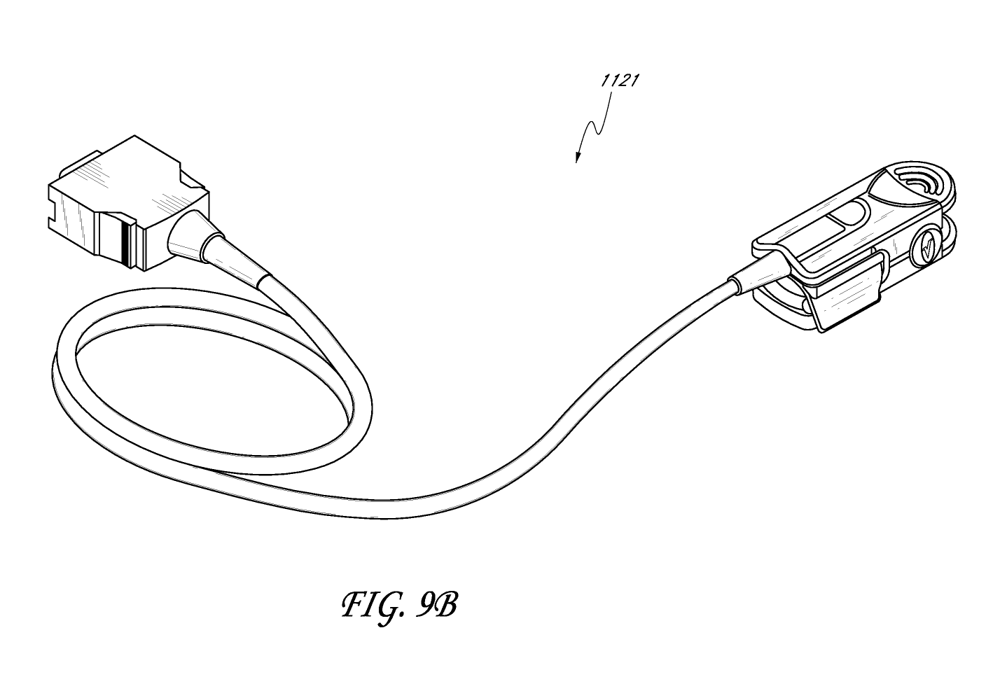

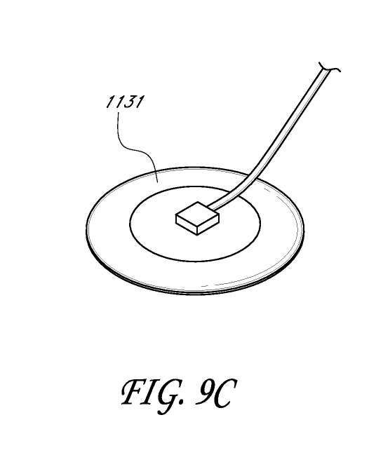

FIG. 9 illustrates an embodiment of a respiratory monitoring system. As shown in FIG. 9, a patient 1101 is monitored using one or more acoustic sensors 1103 which transmit a signal over a cable 1105 to a physiological monitor 1107. The physiological monitor 1107 includes a processor 1109 and, optionally, a display 1111. The acoustic sensor detects biological sounds and vibrations emanating from the throat, chest, or other area of the patient and produces an electrical signal output. The electrical signal output is then processed by the processor 1109. The processor 1109 then communicates information to the display 1111. In an embodiment, the display 1111 is incorporated in the monitor 1107. In an embodiment, the display 1111 is separate from the monitor 1107.

In an embodiment, all of the hardware used to receive and process signals from the acoustic sensor are housed within the same housing. In an embodiment, some of the hardware used to receive and process signals is housed within a separate housing. As used herein, the term “Physiological Monitor” refers to all of the hardware and software, whether in one housing or multiple housing used to receive and process the signals transmitted by the physiological sensor.

In an embodiment, the cable 1105 provides three separate conductors. The three separate conductors include a power line, a ground line, and a signal line. Although described with respect to a three conductor cable, a person of ordinary skill in the art will understand from the disclosure herein that an information element can be accessed over the power conductor independent of the number of other conductors connecting the sensor and the physiological monitoring device. For example, the cable may have four or more conductors including two or more signal lines, and the information element can still be accessed over the power line. In an embodiment, one or more of the conductors is part of the cable's electrical shielding. In an embodiment, information is communicated between the sensor and the monitor wirelessly. In some embodiments, the “ground line” or ground signal refers to an earth ground, but in other embodiments, the “ground line” or ground signal refers to a patient ground, sometimes referred to as a patient reference, a patient reference signal, a return, or a patient return.

In an embodiment, the acoustic sensor is detachable from the respiratory monitoring system to allow for periodic replacement. In an embodiment where a cable is used to connect the sensor and the monitor, the cable is detachable from the respiratory monitoring system and from the sensor to allow for periodic replacement.

In an embodiment, an acoustic sensor 1103 is provided with an information element. In an embodiment, the acoustic sensor 1103 is backward compatible with old, previously installed, or existing physiological monitoring systems. In an embodiment, the information element is accessible over the power line connecting the acoustic sensor 1103 to the physiological monitor 1107. In an embodiment, in order to allow the acoustic sensor 1103 to continue to operate while the information element is accessed over the power line, a power supply is provided to the sensor. In an embodiment, the existing physiological monitoring systems are reconfigured, either in software or hardware, to access the information element on the acoustic sensor.

FIG. 9A illustrates an embodiment of an acoustic sensor 1103. In an embodiment, the acoustic sensor 1103 includes a sensing element, such as, for example, a piezoelectric device or other acoustic sensing device. The sensing element generates a voltage which is responsive to vibrations. In an embodiment, the acoustic sensor includes circuitry configured to transmit the voltage generated by the sensing device to a processor for processing. In an embodiment, the acoustic sensor 1103 includes circuitry for detecting and transmitting information related to biological sounds to a physiological monitor. In an embodiment, the acoustic sensor 1103 includes an information element. Although the present disclosure is described with respect to an acoustic sensor, a person of ordinary skill in the art will understand from the disclosure herein that any type of physiological sensor can be used with the present disclosure. For example, an information element can be used in a pulse oximetry sensor, such as, for example, the pulse oximetry sensor 1121 illustrated in FIG. 9B; an ECG sensor, such as, for example, the ECG sensor 1131 illustrated in FIG. 9C; a blood pressure sensor; or the like.

In an embodiment, the information element is a memory device, such as, for example, an EPROM. In an embodiment, the information element is an impedance value associated with the sensor, such as, for example, a resistive value, an impedance value, or an inductive value. In an embodiment, the information element can be included in the connector (e.g., the cable), the sensor, or a separate housing.

In an embodiment, the information element includes sensor use information which provides information about the use of the sensor. In an embodiment, sensor use information includes information regarding the expiration of the useful life of the sensor, such as, for example, the amount of time the sensor is in use, the number of patients who have used the sensor, the age of the sensor, or the like. In an embodiment, the information element includes information regarding the type and/or identification of the sensor associated with the information element, such as, for example, the manufacturer, the model number, the serial number the patient type (e.g., adult, child, etc.), or the like. In an embodiment, the information element includes manufacturing tolerances and sensing properties, such as, for example, acoustic sensitivity, voltage ranges, current ranges, gain, frequency response, calibration information, or the like. In an embodiment, the sensor stores use information, such as, for example, use time, use temperature, information regarding current use, voltage use, age of the sensor, or the like. In an embodiment, the information element can store patient specific information, such as a patient identification; age, weight, sex, etc. of the patient; the amount of time used on a specific patient; the patient specific problems discovered by the sensor; the user; or the like. In an embodiment, the information element stores information obtained by the sensor before a major event occurs. For example, if a heart attack is detected by the monitor, the information element can store the acoustic information sensed by the sensor for a period of time before the heart attack occurred. In this way, a user can latter review and analyze what the sensor picked up right before the major event occurred. In an embodiment, the monitor uses the sensor information to keep track of which sensors have been attached to the monitor. In an embodiment, the information element is used as a key to upgrade the patient monitor it is connected to.

In an embodiment, the sensor's power supply stores power received from the power line while the power line supplies power. When the power line stops supplying power, the sensor's power supply releases its stored power to the sensing device and the sensing circuitry. This allows the sensing device and the sensing circuitry to continue to operate while the information element is accessed over the power line. In an embodiment, the power supply is a capacitor. In an embodiment, the power supply is a battery. In an embodiment, the power supply is a battery which does not receive power from the monitor power line, but comes fully charged from the manufacturer. In an embodiment, the power supply is a user replaceable battery.

FIG. 10A illustrates an embodiment of a physiological monitoring system incorporating an information element accessible over a power line. The monitor 1201 is connected to at least one sensor 1203. The monitor 1201 includes at least a power interface 1205, a signal interface 1207 and a ground interface 1209. The sensor has a corresponding power interface 1211, signal interface 1213 and ground interface 1215. In some embodiments, the power interface 1211 is referred to as the power line 1211. In an embodiment, the input/output interfaces are connected by connectors 1217 which can be any male/female connectors and/or cables. In one embodiment, the connector 1217 coupled to the power interface 1211 is referred to as a power port 1217, power coupling 1217, or power connector 1217. In an embodiment, the monitor 1201 and the sensor 1203 communicate wirelessly. In an embodiment, the monitor 1201 includes a switch 1227, a power supply interface 1229, an information signal interface 1231, and a processor 1233. In an embodiment, the sensor 1203 includes an information element 1221, a secondary power supply 1223 and a sensing circuitry/device 1225.

In operation, power is supplied to the sensor 1203 from the monitor 1201 through the power interfaces 1229, 1205, 1211. The power is supplied to the information element 1221, the secondary power supply 1223 and the sensing circuitry/device 1225. The sensing circuitry/device 1225 measures a physiological parameter and outputs an indication of the physiological parameter to the signal interfaces 1213, 1207, to the monitor 1201 and processor 1233. Sensing circuitry/device 1225 can be any type of physiological monitor system capable of monitoring a physiological characteristic, such as, for example, biological sounds, blood parameters, cardiac signals, blood pressure, or the like.

In one embodiment, the information element 1221 is silent until accessed by the monitor 1201 over the power interface 1211. The monitor 1201 activates the switch 1227 which switches the power interface 1205 from connecting with the power interface 1229 to connecting with the information signal interface 1231. At this point, the monitor stops providing power to the sensor 1203. The information element 1221 then communicates with the monitor over the power interface. However, the sensor 1203 continues to monitor physiological parameters and transmits information to the monitor 1201 and processor 1233. The secondary power supply 1223 supplies power to the sensing circuitry/device 1225 while the monitor communicates with the information element. Throughout the information element communication process, the processor 1233 continues to receive and process the signals and send the processed information to the display. A user utilizing the present system is generally unaware that the process is occurring because there is no break in the acquisition of physiological information. In an embodiment, the monitor displays an indication that the information element is being accessed.

FIG. 10B illustrates an embodiment of a physiological monitoring system incorporating an information element into a cable. As illustrated in FIG. 10B, a cable 202 is used to connect the monitor 1201 with the sensor 1203. The cable 202 includes an information element 1221 which is accessed over the power conductor. In an embodiment, sensor 1203 does not include an information element, but does include a secondary power supply. In an embodiment, the sensor 1203 includes an information element and a secondary power supply. In an embodiment, the cable includes both an information element 1221 and a secondary power supply 1223 and the sensor 1203 does not include either an information element or a secondary power supply. Those of skill in the art will appreciate from the disclosure herein, that any combination of the above described elements are possible. For example, in an embodiment, the information element and secondary power supply can be included in both the cable and the sensor. The drawings and descriptions of the above described combinations is made by way of example, and not limitation.

FIG. 10C illustrates a circuit diagram of an embodiment of a physiological monitoring system incorporating an information element accessible over a power line. The monitor 1201 of the embodiment of FIG. 10C includes a voltage power supply 1241 and resistor 1243. The monitor also includes a power sink device 1245, such as, for example, a transistor, such as for example, a field effect transistor, bipolar junction transistor or the like. The power sink 1245 is used to pull the voltage power supply substantially to ground to cause a “low” or zero signal across the power line. The monitor also includes a one way communication device 1247, such as, for example, a diode. The one way communication device 1247 communicates the voltage level of the power line to the monitor. The monitor 1201 also has a signal input interface 1207 and a ground interface 1209 for connection with the sensor 1203. Signal interface 1207 connects the signal output of the sensor 1203 with the processor 1233. The processor 1233 processes the signals and sends information to a display.

In an embodiment, the processor 1233 is configured to extract information regarding various physiological phenomena and conditions from the sensor signal. For example, in an embodiment, the process is configured to determine one or more of inspiratory time, expiratory time, inspiratory to expiratory ratio, inspiratory flow, expiratory flow, tidal volume, minute volume, apnea duration, breath sounds—including rales, rhonchi, or stridor, changes in breath sounds, heart rate, heart sounds—including S1, S2, S3, S4, or murmurs, or changes in heart sounds.

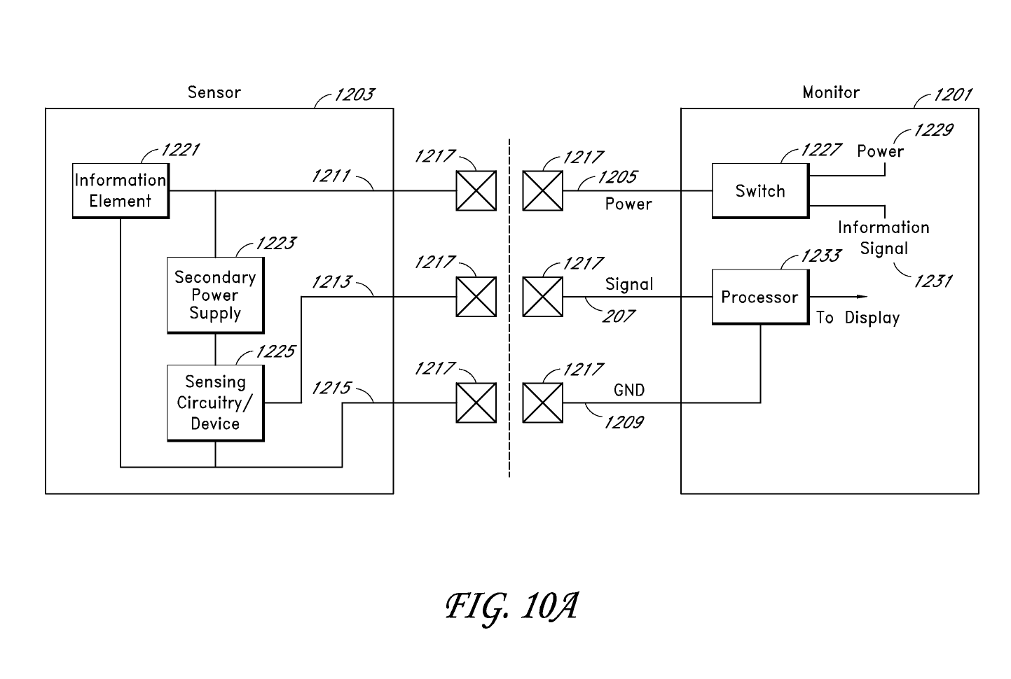

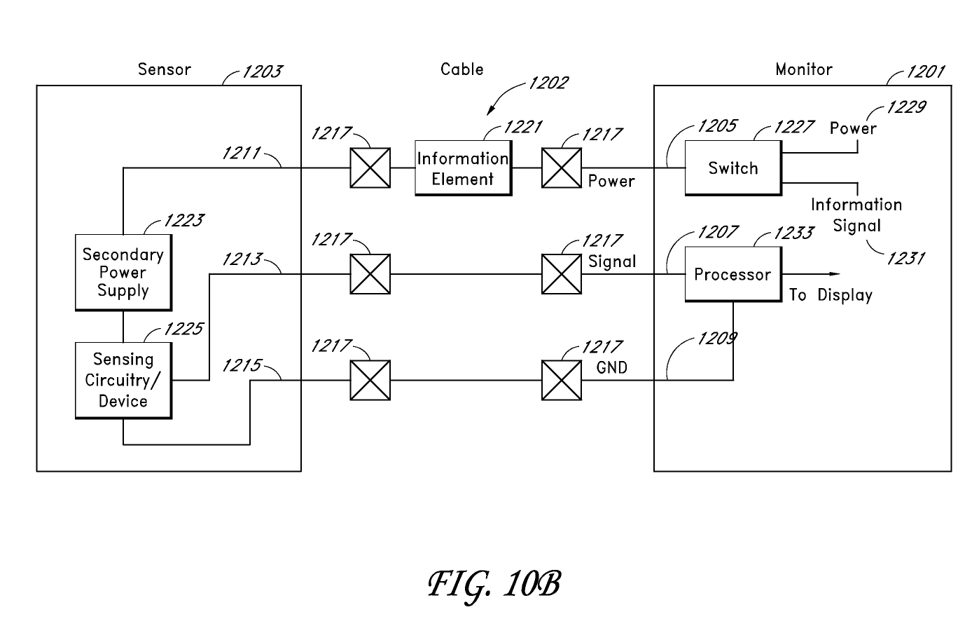

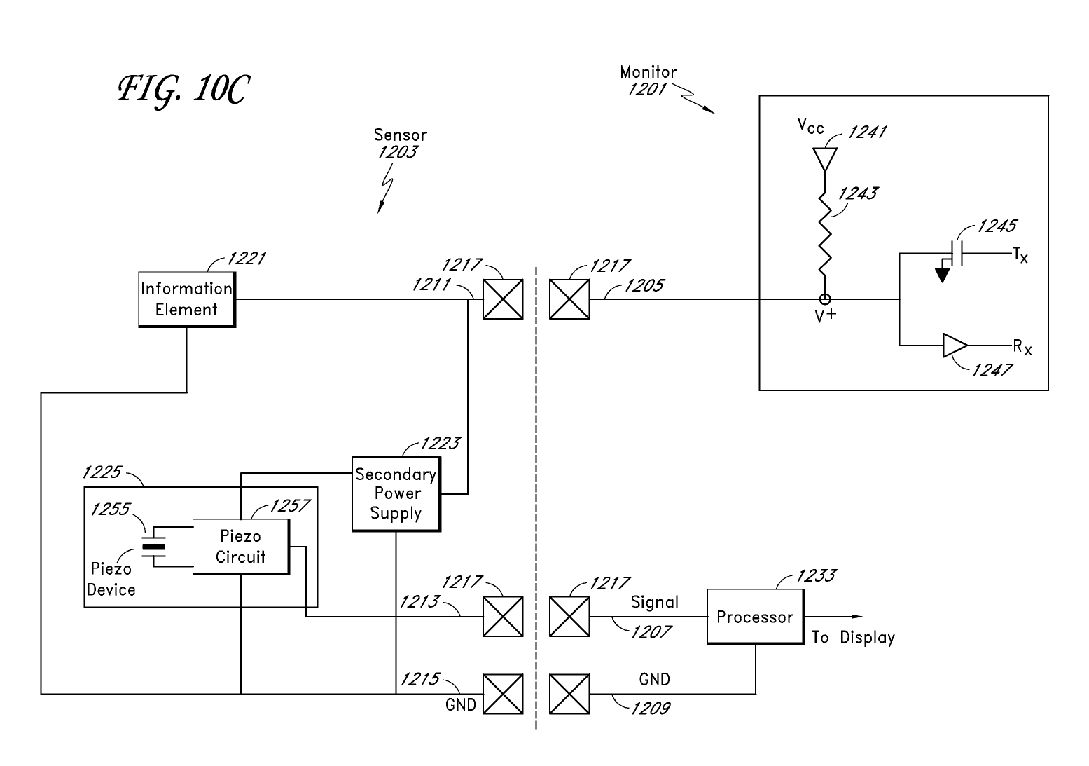

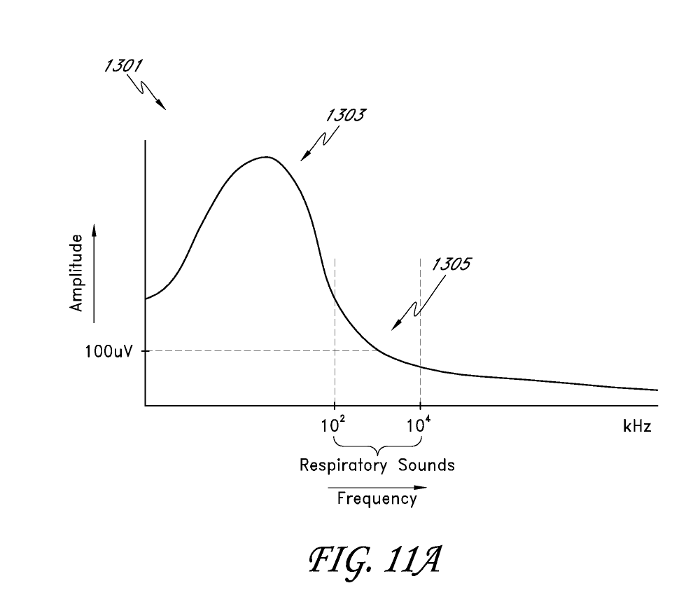

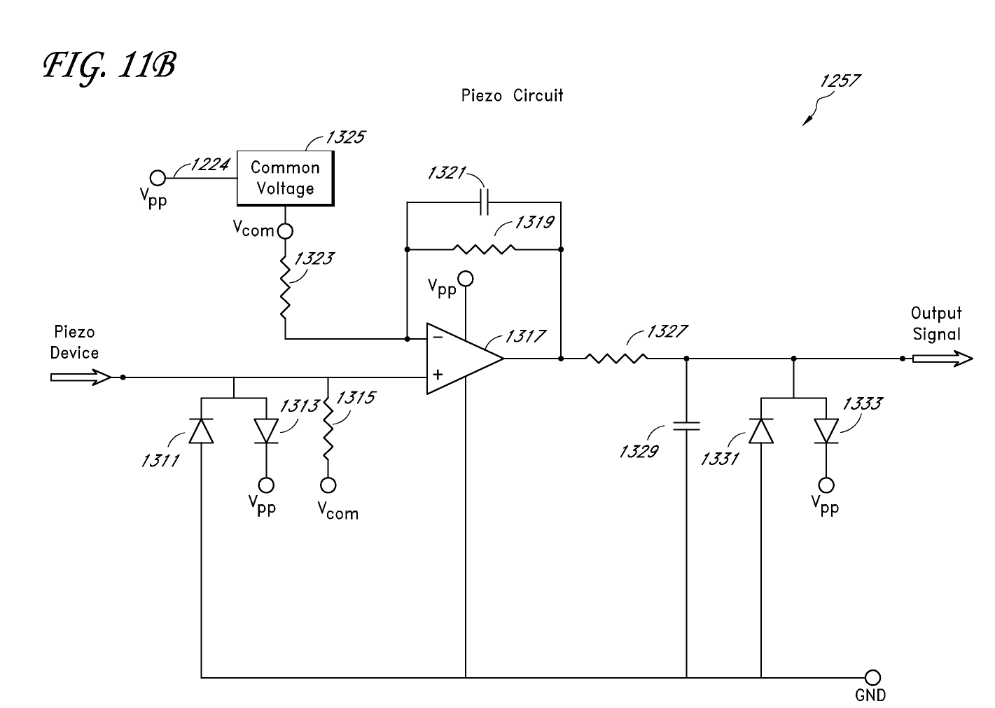

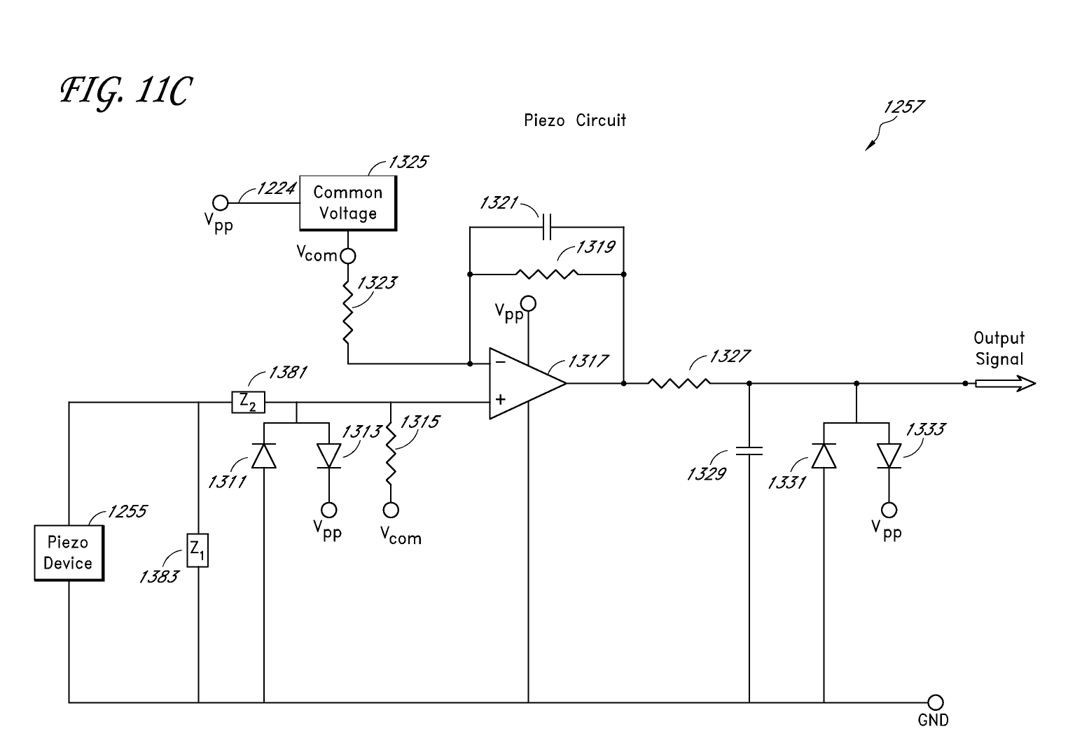

Referring still to FIG. 10C, in an embodiment, the sensor 1203 includes information element 1221, power interface 1211, secondary power supply 1223 and sensing circuitry/device 1225. In an embodiment, the sensing circuitry/device 1225 includes a piezoelectric sensing element 1255 and a piezo circuit 1257. The piezoelectric sensing element 1225 senses vibrations and generates a voltage in response to the vibrations. The vibrations generated by the sensing element 1225 are then communicated to the piezo circuit 1257 which conditions the signal and transmits the signal over signal interface 1213 to the processor 1233 for processing. The piezo circuit 1257 is described in further detail in relation to FIG. 11B below.

In operation, the monitor 1201 is able to communicate with the information element 1221 of the sensor 1203 similar to the process described in relation to FIG. 10A. During the first mode of operation, or the power supply mode, the power is supplied from the monitor 1201 to the sensor 1221 through power interface 1211. The information element 1221 is silent during this mode.

When the monitor 1201 wants to access the information element 1221, the monitor “pings” the sensor 1203 by temporarily sinking the power supply output using power sink 1245. This is done buy applying a voltage to line Tx sufficient to turn on the power sink 1245 and sink the power supply to zero. At this point, power is no longer supplied to the sensor 1203. This effectively pings the information element 1221, and communicates a command to begin communication with the monitor 1201. It is to be understand from the disclosure herein that more complicated communication protocols could also be used, for example, a series of pings could be used to initiate communications.

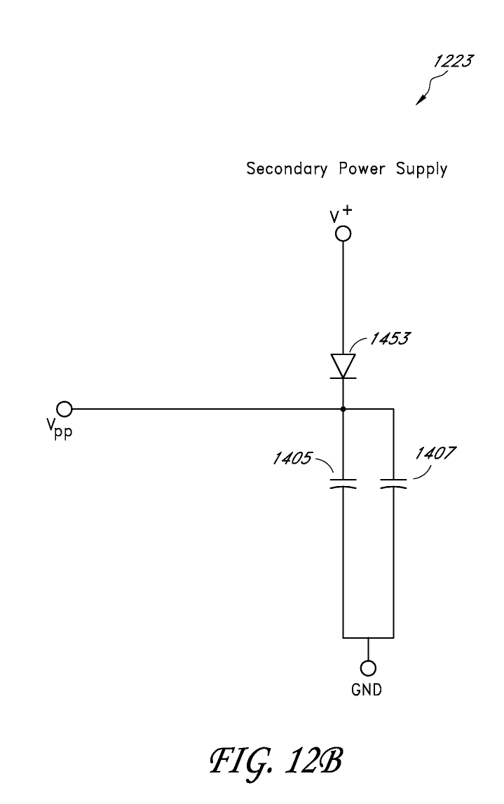

While the power line is low and power is not being supplied to the sensor 1203, the secondary power supply 1223 begins supplying power to the sensing circuitry/device 1225, which continues to monitor the patient. The secondary power supply's operation is explained in greater detail in relation to FIGS. 12B-12C below. While the secondary power supply 1223 supplies power to the rest of the sensor 1203, the monitor 1201 is free to communicate with the information element 1221.

In response, the information element 1221 communicates information by similarly driving the power line 1211 low for each bit of communication. The monitor 1201 receives the communications through the diode 1247 to line Rx. In an embodiment, the information received by the monitor 1201 is sent to the processor 1233 for analysis or sent to a separate processor. In an embodiment, the processor 1233 can be a single processor or multiple separate processors.

As described above, the information element 1221 is one part of the sensor 1203. The sensor 1203 also includes circuitry and/or devices for obtaining physiological information from the patient. In an embodiment, the sensor 1203 is an acoustic sensor. In an embodiment, the sensor 1203 is one or more of an acoustic sensor, an optical sensor, an ECG sensor, a blood pressure sensor, or the like.

In an embodiment, an acoustic sensor, including a piezoelectric device, configured to sense acoustic parameters of a patient. FIG. 11A illustrates an example of a frequency response 1301 of an embodiment of the piezoelectric device 1255. The frequency response 1301 includes a large low frequency response near point 1303 and then diminishes at higher frequencies. Many respiratory important noises occur near point 1305 at frequencies between about 10² and 10⁴ kHz. In an embodiment, due to the unwanted low and high frequency response, some conditioning of the signal is done before it is sent to the monitor 1201 for analysis.

FIG. 11B illustrates an embodiment of a circuit diagram for use with a piezoelectric circuit 1257 for conditioning the piezoelectric device signal. The piezoelectric circuit includes diodes 1311, 1313, a resistor 315, an operational amplifier (“op amp”) 1317, a resistor 1319, a capacitor 1321, a resistor 1323, a common voltage supply 1325, a resistor 1327, a capacitor 1329, and diodes 1331, 1333.

In an embodiment, diodes 1311, 1313, 1331, 1333 provide electrostatic discharge (ESD) protection. In an embodiment, if the voltage at the diodes goes above about 0.7 volts below V⁺, also referred to herein as V_{CC} or the power supplied by the monitor, or below about 0.7 volts above ground, the voltage is clamped or at least partially discharged so that the rest of the circuit is not affected. This provides valuable protection against, for example, a defibrillator shock. Those of skill in the art will appreciate that other ways of protecting the circuit from ESD can also be used with the present disclosure. For example, different clamping voltages can be used or a different ESD protection circuit design can be used.

In an embodiment, common voltage 1325 and resistor 315 provide a mid-level voltage DC offset, Vcom, for the piezoelectric signal to ride or to be superimposed or added to. In one embodiment, the mid-level voltage is about 2.5 volts. The mid-level voltage prevents the piezoelectric signal from being clamped by the diodes 1311 and 1313. The mid-level voltage also provides a system where a negative power supply is not needed to operate the op amp 1317 because the signal generally stays positive. The time-varying voltage provided by the piezoelectric device is added to the substantially constant voltage provided by Vcom to create a DC offset to the piezo signal. Those of skill in the art will appreciate that other ways of providing a DC offset can also be used with the present disclosure.

In an embodiment, resistor 1315 in conjunction with the inherent capacitance of the piezoelectric device 1255 provide a high pass filter, which eliminates unwanted low frequencies. In an embodiment, the high pass filter filters frequencies below about 100 Hz. In an embodiment, an additional capacitor is inserted between the piezoelectric device and ground or between the piezoelectric device and the resistor 1315 in order to provide a high pass filter. Those of skill in the art will appreciate that other ways of providing filtering can also be used with the present disclosure. In an embodiment, a low pass filter can used in conjunction with or instead of the high pass filter.

In an embodiment, op amp 1317 is configured to provide gain to the piezoelectric signal. In an embodiment, the op-amp 1317 is configured in a non-inverting configuration. In an embodiment, the gain of the op amp 1317 is configured to be about 2 for desired frequencies as determined by the capacitor 1321 and resistor 1319. In an embodiment, the gain of op amp 1317 is 2 for frequencies below about 10-15 kHz, and 1 for frequencies above about 10-15 kHz. Those of skill in the art will appreciate that other ways providing amplification can also be used with the present disclosure.

In an embodiment, resistor 1327 and capacitor 1329 provide a low pass filter on the output of the op amp 1317. In an embodiment, the low pass filter filters out frequencies above about 10-15 kHz. Those of skill in the art will appreciate that other ways of providing a low pass filter can also be used with the present disclosure. In an embodiment, a high pass filter is also provided on the output in addition to or instead of the low pass filter.

In operation, in the embodiment of FIG. 11B, a piezoelectric device provides a signal to the piezo circuit 1257 carried on the mid-level DC offset voltage supplied by the common voltage 1325 and the resistor 1315. The signal is high-pass filtered and then amplified by the op amp configuration. The signal is then low-pass filtered and outputted for communication to the processor for further processing and analysis. In an embodiment, the piezoelectric device signal is low pass filtered before being amplified by the op amp configuration. In an embodiment, the signal is high pass filtered after it is outputted from the op amp configuration.

FIG. 11C illustrates a circuit diagram of an embodiment of a piezoelectric circuit with impedance compensation. Impedances Z₁ 1383 and Z₂ 1381 are used to control the signal level strength and frequency of interest input to the op amp 1317. In one embodiment, the impedances are used to minimize the variation of the piezo device 1255 signal output. In an embodiment, only one impedance, either Z₁ 1383 or Z₂ 1381 are used. In one embodiment, bot impedances Z₁ 1383 and Z₂ 1381 are used. Impedance Z₁ 1383 and Z₂ 1381 can be constructed of any combination of impedances, including resistive, capacitive and inductive impedances. In one embodiment, an RC circuit is used as the impedance. In one embodiment, an RLC circuit is used as the impedance. In one embodiment, only a capacitor is used as the impedance. In one embodiment a resistor and a capacitor are used in series. In one embodiment a resistor is used in parallel with a capacitor. In one embodiment, impedances Z₁ 1383 and Z₂ 1381 are constructed of different types of impedances. In one embodiment, impedances Z₁ 1383 and Z₂ 1381 are constructed of the same type of impedance. As one of skill in the art would understand from the disclosure herein, any combination of impedance can be used depending on the frequency of interest.

FIG. 12A illustrates a circuit diagram of an embodiment of an information element 1221. The information element 1221 includes a memory device and/or controller 1401, a power sink 1402, such as, for example, a transistor, and an optional diode 1403. The transistor can include an FET, JFET, CMOS, or bipolar transistor.

FIG. 12B illustrates a circuit diagram of an embodiment of a secondary power supply 1223, such as illustrated in FIGS. 10A-10C. The secondary power supply 1223 includes capacitor 1405 and capacitor 1407. The capacitors 1405, 1407 store energy or power when power is supplied from the monitor and discharge the stored energy or power when energy or power is not being supplied by the monitor. The capacitor 1405 provides a fast but relatively short discharge, while capacitor 1407 provides a slow but relatively long discharge. Thus, when power supplied from the monitor is turned off, the fast response capacitor 1405 quickly provides power to the rest of the sensor, while the slow response capacitor 1407 continues to provide power to the rest of the sensor after the fast response capacitor 1405 has released all of its stored energy. The fast response capacitor 1405 can have a capacitance of about 0.01 μF and the slow response capacitor 1407 can have a capacitance of about 0.1 μF. In one embodiment, the slow response capacitor 1407 has a capacitance of about ten-times the capacitance of the fast response capacitor 1405. In other embodiments, the capacitance of the slow response capacitor 1407 is about 5-10 or more than 10 times the capacitance of the slow response capacitor 1407. Furthermore, in one embodiment, the fast response capacitor 1405 allows continuous, un-interrupted operation of the sensor 1203 as it changes modes from receiving power from the physiological monitor to communicating information with the physiological monitor over the power line 1211, or through its power port 1217. When the secondary power supply begins to supply power to the sensing circuitry/device 1225, diode 1453 prevents the power supplied by the secondary power supply from interfering with communications between the monitor 1201 and the information element 1221. Those of skill in the art will understand from the disclosure herein that one, two, three or more capacitors can be used to provide secondary power to the sensing circuitry/device 1225.

FIG. 12C illustrates the voltage response of the secondary power supply 1223. Power is supplied as represented by V⁺ 1481. As shown, V⁺ 1481 is pulled low at t_{off} 1482. Secondary power 1483 begins to be supplied at t_{off} 1482 as well. As shown in FIG. 12C, the power stored in the secondary power supply 1223 decrease with time, but continues to discharge a power supply sufficient to operate the rest of the sensor circuitry. At tₒₙ 1484, V⁺ 1481 is restored an the secondary power supply 1223 as represented by the secondary power 1483 is replenished.

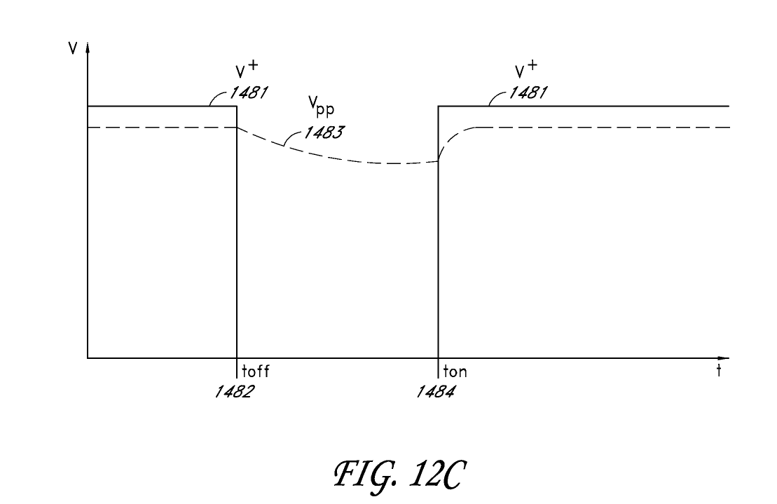

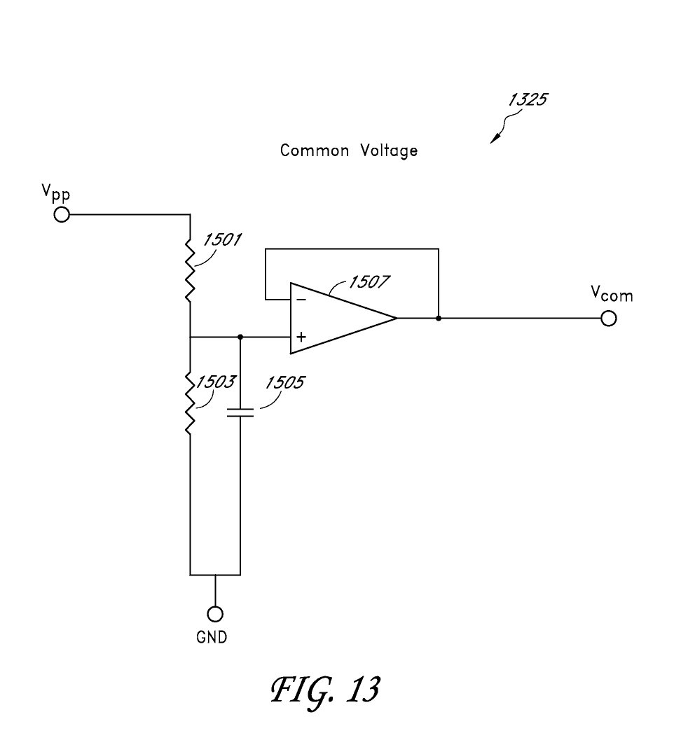

FIG. 13 illustrates a circuit diagram of a common voltage supply 1325, such as illustrated in FIG. 11B. The common voltage supply 1325 provides a voltage follower circuit which isolates the voltage output from the op amp 1507. The common voltage supply 1325 includes resistors 1501, 1503, capacitor 1505 and op amp 1507. The resistors 1501, 1503 and capacitor 1505 are provided to the op amp 1507 in order to provide the correct circuit configuration to provide a common voltage power supply.

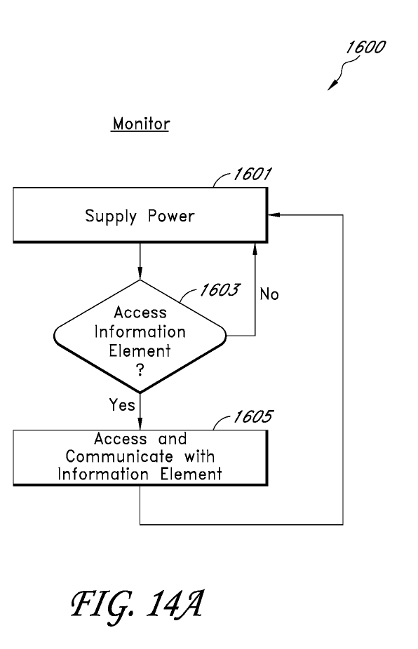

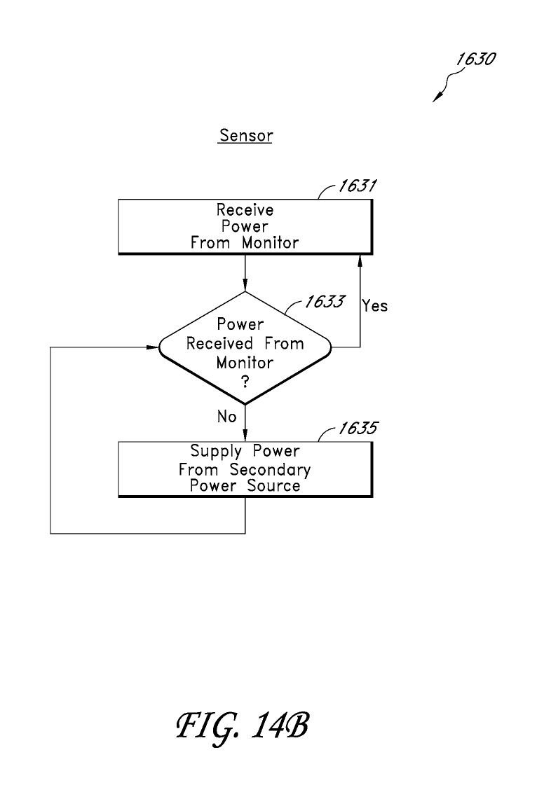

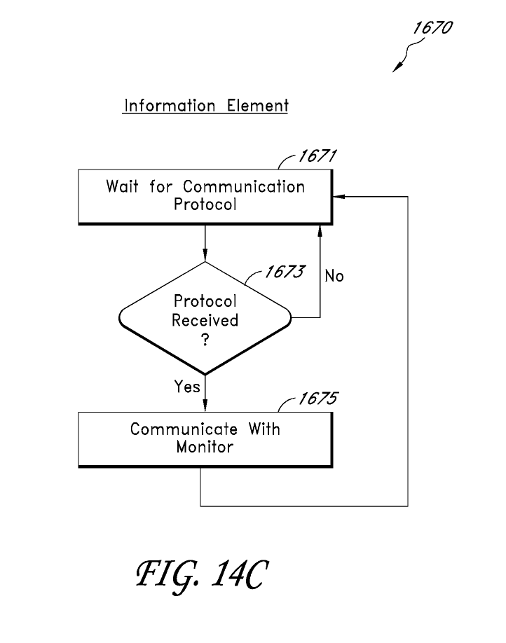

FIG. 14A illustrates a flowchart of an embodiment of a method 1600 performed by a physiological monitor in order to communicate over a power conductor with an information element. The process 1600 begins at block 1601 where the monitor supplies power. At decision block 1603, the monitor decides whether to access the information element. The decision of whether to access the information element can be based on many different factors, such as, for example, whether the monitor has previously accessed the information element; the time since the last access; whether there has been an event that triggers the access, such as, for example, power on, power off, a physiologically important event, or the like; whether a user has requested the monitor to access the information element; or the like. If the answer at decision block 1603 is no, then the process 1600 returns to block 1601. If the answer at decision block 1603 is yes, then the process 1600 moves to block 1605. At block 1605, the monitor accesses and communicates with the information element, and once communication is complete, the process 1600 returns to block 1601.

FIG. 14B illustrates a flowchart of an embodiment of a method 1630 performed by a physiological sensor in order to continue to power itself when the monitor stops supplying power. The process 1630 begins at block 1631 where the sensor receives power from the monitor 1631. The process 1630 then moves to decision block 1633 where the sensor decides whether power is being received from the monitor. If the answer at decision block 1633 is yes, then the process 1630 returns to block 1631. If the answer at decision block 1633 is no, then the process 1630 moves to block 1635 where the sensor supplies power to itself from the secondary power supply source. The process 1630 then returns to decision block 1633.

FIG. 14C illustrates a flowchart of an embodiment of a method 1670 performed by an information element in communicating with a monitor. The process 1670 begins at block 1671 where the information element waits for the communication protocol to occur. The process 1670 then moves to decision block 1673, where the information element determines whether the communication protocol has been received. If the answer is no at decision block 1673, then the process 1670 returns to block 1671. If the answer is yes, then the process 1670 moves to block 1675 where the information element communicates with the monitor. After communication is complete at block 1675, the process 1670 returns to block 1671.

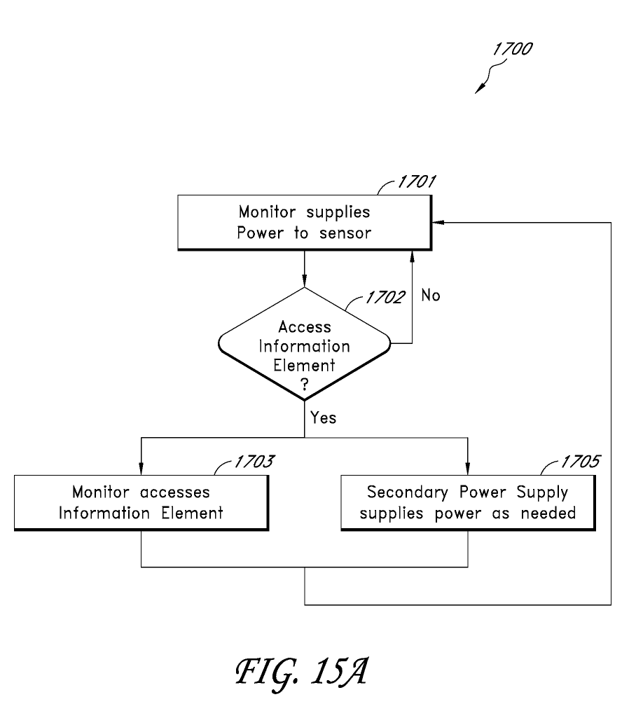

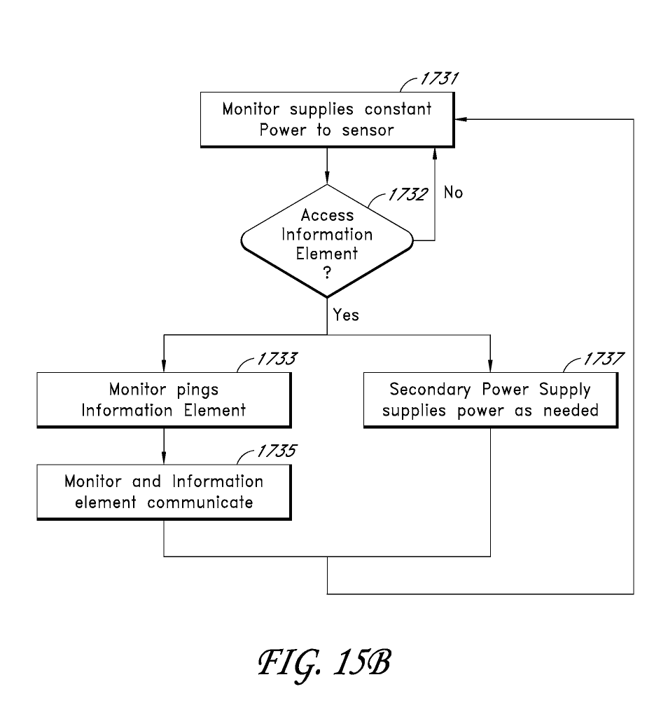

FIG. 15A illustrates a flowchart of an embodiment of a method 1700 performed by a physiological monitoring system incorporating an information element accessible over a power interface. The information element/monitor communication process 1700 begins at block 1701 where the monitor supplies power to the sensor. The process 1700 then moves concurrently to block 1703 and block 1705. At block 1703 the monitor accesses and communicates with the information element 1703. At block 1705, the secondary power supply supplies power as needed to the rest of the sensor circuit while communication is occurring. After the communication between the monitor and the information element is finished, the process 1700 then returns to block 1701.

FIG. 15B illustrates a flowchart of another embodiment of a method of accessing an information element over a power interface. The process begins at block 1731 where the monitor supplies constant power to the sensor. The process then moves concurrently to block 1733 and block 1737. At block 1733, the monitor pings the information element. Once communication between the monitor and the information element is established at block 1733, the system moves to block 1735. At block 1735, the monitor and information element communicate. At the same time, at block 1737, the secondary power supply supplies power as needed to the rest of the sensor circuitry. After communication is completed between the monitor and the information element, the process then returns to block 1731.

FIG. 16A illustrates an embodiment of an information element communication process 1800. The process 1800 begins at block 1801 where the information element waits for a read request. The process 1800 moves to decision block 1803 where the process 1800 determines whether a read request has been received. If a read request has not been received at decision block 1803, the process 1800 returns to block 1801. If a read request has been received at block 1803, then the process 1800 moves to block 1805 where the information element transmits stored data. The process 1800 then moves to decision block 1807 where the process 1800 determines if the data transmission is complete. If data transmission is not complete at decision block 1807, then the process 1800 returns to block 1805. If the data transmission is complete at decision block 1807, then the process 1800 returns to block 1801.

FIG. 16B illustrates a process 1850 for requesting and receiving data from an information element over a power line. The process 1850 begins at supply power block 1851 where power is supplied over the power line. The process 1850 then moves to decision block 1853 where the process 1850 determines if it should send a read request. If the decision at decision block 1853 is no, then the process 1850 returns to supply power block 1851. If the decision at decision block 1853 is yes, then the process moves to block 1855 where the process 1850 sends a read request to the information element. The process 1850 then moves to block 1857 where data is received from the information element. The process 1850 then moves to decision block 1859 where the process 1850 determines if data transmission is complete. If data transmission is complete, then the process moves to block 1851. If the data transmission is not complete, then the process 1850 returns to block 1857.

FIG. 17A illustrates another embodiment of an information element communication process 1900. The process 1900 begins at block 1901 where the information element waits for a read or write request. The process 1900 then moves to decision block 1903 where the process 1900 determines whether a read request has been received. If a read request has been received at block 1903, then the process 1900 moves to transmit data block 1905 where the information element transmits stored data. The process 1900 then moves to decision block 1907 where the process 1900 determines if the data transmission is complete. If data transmission is not complete at data transmission decision block 1907, then the process 1900 returns to transmit data block 1905. If the data transmission is complete at data transmission decision block 1907, then the process 1900 returns to wait for read or write request at block 1901.

If at decision block 1903, a read request has not been received, the process 1900 moves to decision block 1909. At decision block 1909, the process 1909 determines if a write request has been received. If a write request has not been received, then the process 1900 returns to block 1901. If a write request has been received, then the process 1900 moves to block 1911 where data is received and stored in memory. The process 1900 then moves to decision block 1913 where the process 1900 determines if data transmission and storage is complete. If data transmission and storage is complete, then the process 1900 moves to block 1901. If data transmission and storage is not complete, then the process returns to block 1911.

FIG. 17B illustrates a process 1950 for receiving or writing data to an information element over a power line. The process 1950 begins at supply power block 1951 where power is supplied over the power line. The process 1950 then moves to decision block 1953 where the process 1950 determines if it should send a read request. If the decision at decision block 1953 is yes, then the process moves to block 1955 where the process 1950 sends a read request to the information element. The process 1950 then moves to block 1957 where data is received from the information element. The process 1950 then moves to decision block 1959 where the process 1950 determines if data transmission is complete. If data transmission is complete, then the process moves to block 1951. If the data transmission is not complete, then the process 1950 returns to block 1957.

If at block 1953, the decision is no, the process 1950 moves to decision block 1961 where the process 1950 determines if it should send a write request. If the decision at decision block 1961 is no, then the process 1950 returns to block 1951. If the decision is yes, then the process 1950 moves to block 1963 where a write request is sent. The process 1950 then moves to block 1965 where data is transmitted to the information element. The process 1950 then moves to decision block 1967 where the process 1950 determines if the transmission is complete. If the transmission is not complete, the process 1950 returns to block 1965. If transmission is complete then the process 1950 moves to block 1951.

In many cases, it is advantageous to provide information acquisition control with any of the physiological monitoring systems described herein. Such control systems, in many cases, can be used to dynamically adjust the gain of the sensing device (e.g., piezoelectric sensor or microphone), to accommodate changes in input signal amplitudes.

In various embodiments, acoustic signal processing systems are systems that monitor acoustic signals generated by a medical patient and process the signals to determine any of a variety of physiological parameters of the patient. For example, in some cases, an acoustic signal processing system is an acoustic respiratory monitor. An acoustic respiratory monitor can determine any of a variety of respiratory parameters of a patient, including respiratory rate, inspiratory time, expiratory time, i:e ratio, inspiratory flow, expiratory flow, tidal volume, minute volume, apnea duration, breath sounds, rales, rhonchi, stridor, and changes in breath sounds such as decreased volume or change in airflow. In addition, in some cases the acoustic signal processing system monitors other physiological sounds, such as heart rate to help with probe off detection, heart sounds (e.g., S1, S2, S3, S4, and murmurs), and changes in heart sounds such as normal to murmur or split heart sounds indicating fluid overload. Moreover, the acoustic signal processing system may use a second probe over the chest for better heart sound detection, keep the user inputs to a minimum (for example, only input height), and use an HL7 interface to automatically input demography.

Acoustic signal processing systems generally include a sensor, a gain adjustment stage, an analog-to-digital converter, and a processor. In various embodiments other components are also included, such as filters, displays, controllers, and/or isolators, as described in greater detail below.

In certain embodiments, acoustic information is received by a sensor which converts the acoustic information into a voltage signal. The voltage signal is transmitted to a bank of amplifiers in parallel with one another. These amplifiers may have different gain levels. In one embodiment, a low gain amplifier is in parallel with a high gain amplifier. Each amplifier receives the voltage signal and outputs an amplified voltage signal to one or more analog-to-digital converters, which in turn transmit digital signals corresponding to each amplifier output to a processor. One such suitable sensor is described in U.S. Provisional No. 60/893,858, filed Mar. 8, 2007, which is incorporated by reference herein. In addition, U.S. Provisional No. 60/893,850, filed Mar. 8, 2007, and U.S. Provisional No. 60/893,853, filed Mar. 8, 2007, are also incorporated by reference herein.

The processor in various embodiments constructs an output signal by evaluating the two digital input signals. First the processor determines if a sample from the high gain amplifier signal is clipping. If the sample is not clipping, the processor selects that sample. However, if the sample is clipping, the processor selects a corresponding sample from the low gain amplifier signal. The processor then multiplies the sample from the low gain amplifier by a compensation factor. In this manner, the processor constructs an output signal including samples from both the low and high gain amplifier signals.

The processor of certain embodiments automatically calibrates one or more digitally-controlled amplifiers. For example, in one embodiment having two amplifiers, output signals from the low and high gain amplifiers are transmitted to low and high gain digitally-controlled amplifiers. The low and high gain amplifiers amplify the signals to a voltage level determined by the processor. The output from the low gain digitally-controlled amplifier in some embodiments is used by the processor as a baseline calibration level. Using the baseline calibration level, the processor determines whether a certain number of least significant bits (LSBs) are changing on the output of the high gain digitally-controlled amplifier. If the number of changing LSBs exceeds a threshold value, the processor calibrates the low and high gain digitally-controlled amplifiers by adjusting their gains accordingly.

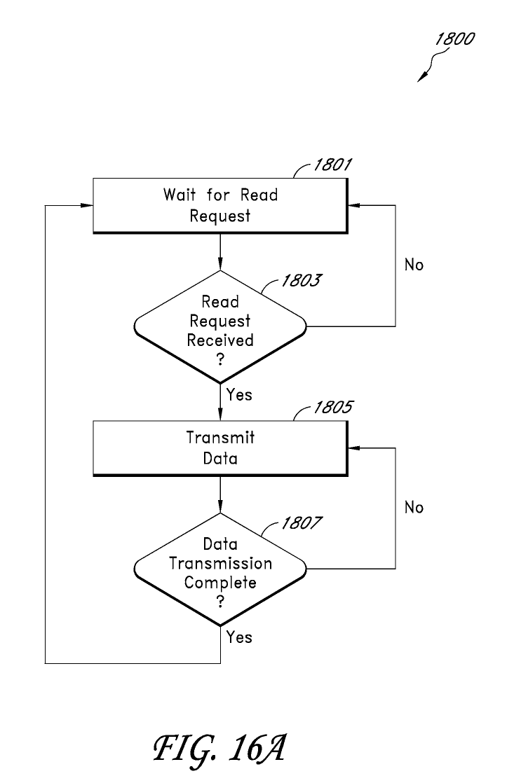

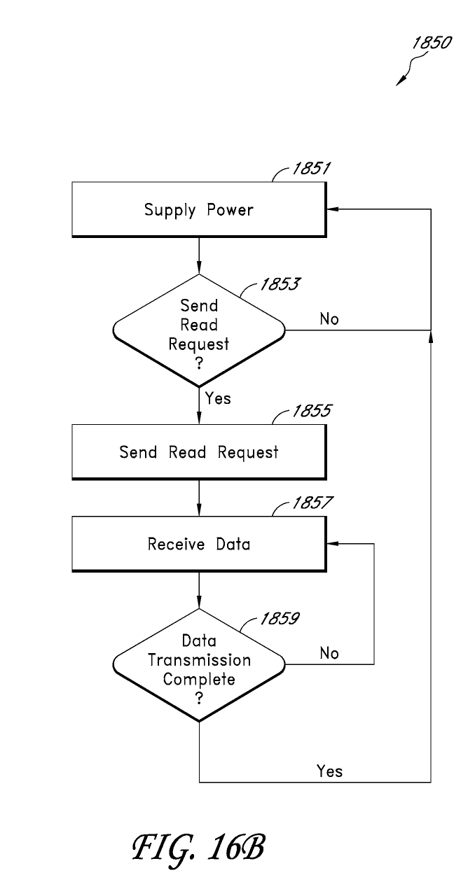

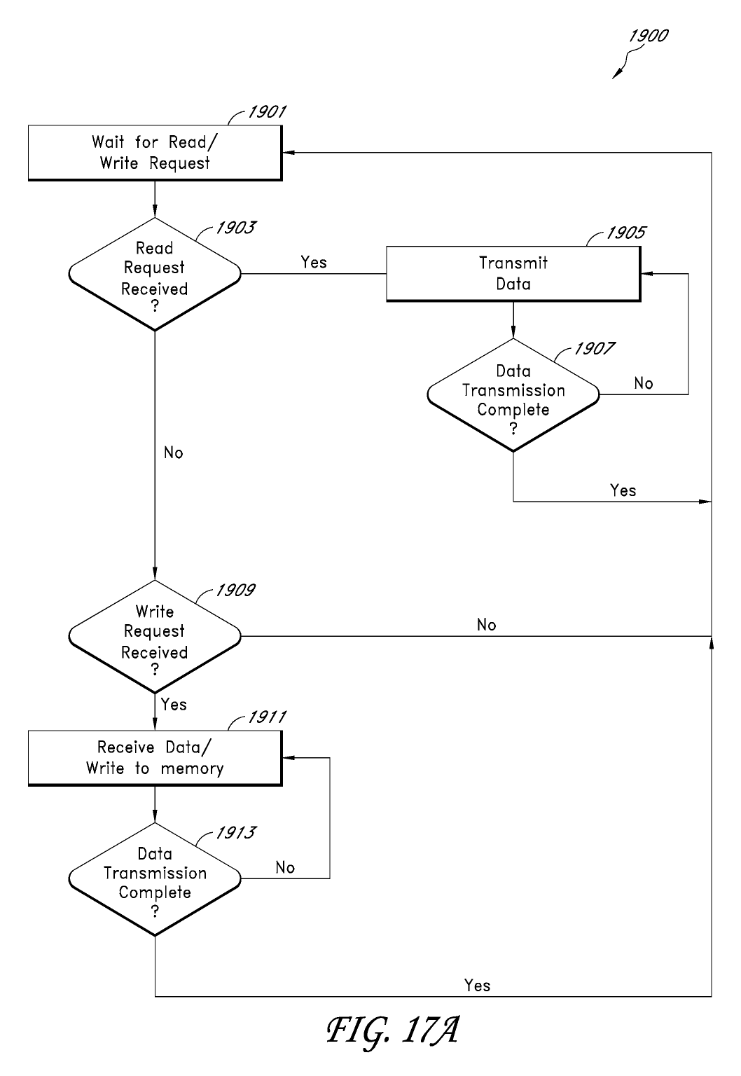

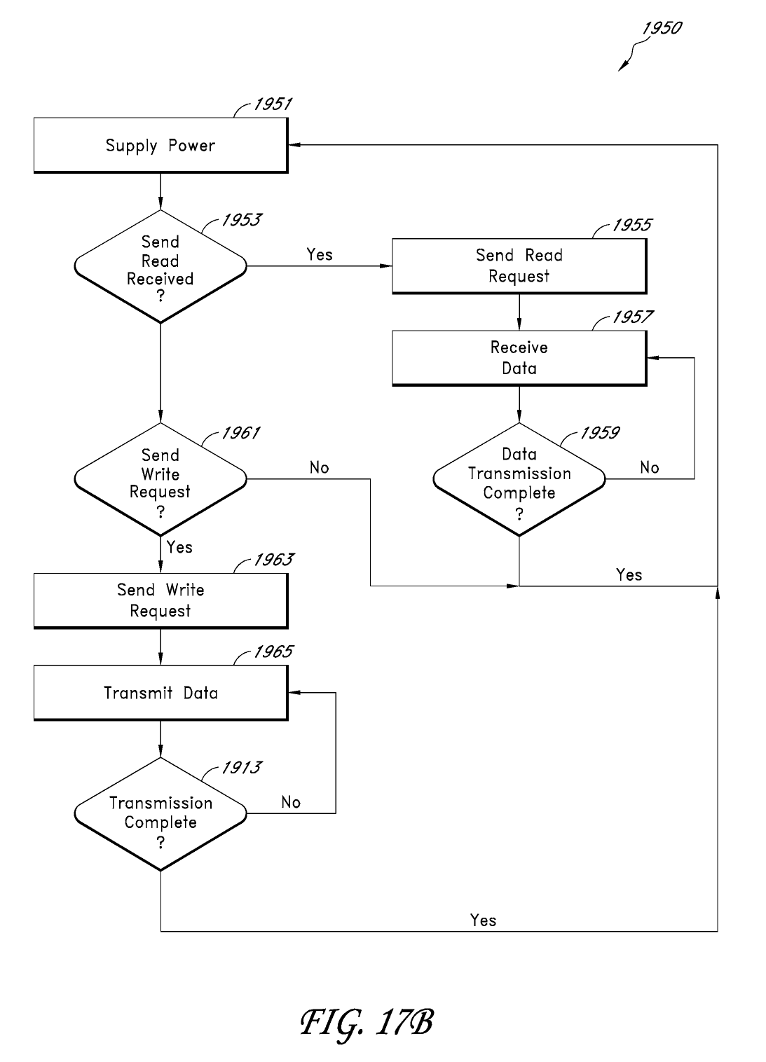

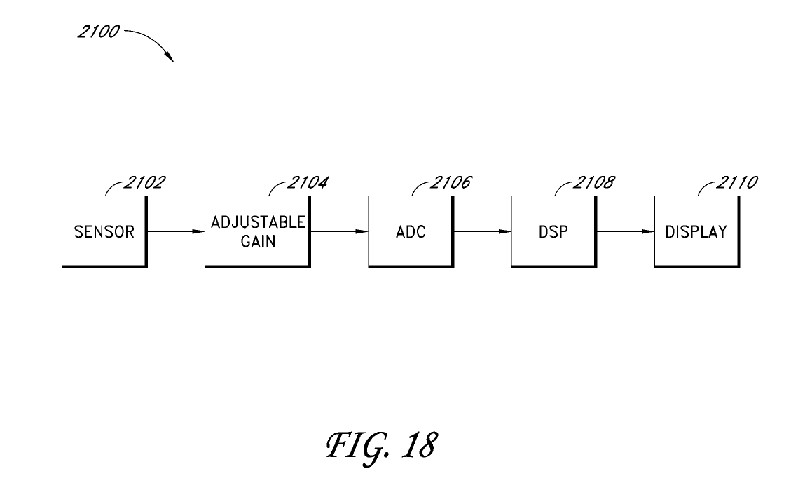

Referring to FIG. 18, certain embodiments of an acoustic signal processing system 2100 include a sensor 2102 that monitors physiological sounds from a patient. These physiological sounds may include heart, breathing, and digestive system sounds, in addition to many other physiological phenomena. Sensor 2102 in certain embodiments is a biological sound sensor, such as a sensor described in U.S. Pat. No. 6,661,161, which is hereby incorporated by reference. Sensor 2102 or possibly multiple sensors 2102 outputs a voltage signal composed of time-varying voltages to an adjustable gain stage 2104. In alternative embodiments, the sensor 2102 outputs an optical, wireless, or other type of signal. Accordingly, wires, buses, channels, and other electrical contacts described herein may be replaced with or additionally include fiberoptic cable, antennas, waveguides, and the like.

The adjustable gain stage 2104 amplifies the voltage signal to an appropriate level for analog to digital conversion and for later digital signal processing. In certain embodiments, the adjustable gain stage 2104 automatically adjusts the amplification or gain level of the voltage signal, without intervention by a human operator, in situations where the voltage signal reaches a high voltage or exceeds a predetermined threshold level. Such situations might occur when a patient talks, coughs, or snores, where the loud sound of talking, coughing, or snoring creates a correspondingly high voltage in the sensor 2102. In such cases, an amplifier with a non-adjustable gain might saturate and thereby lose information concerning the patient's breathing pattern. Accordingly, the adjustable gain stage 2104 overcomes this problem by automatically compensating for the high voltage signals.

An analog-to-digital converter (ADC) 2106 receives the amplified voltage signal from the adjustable gain stage 2104. In certain embodiments, the ADC 2106 is a delta-sigma converter, such as the delta-sigma converter described in U.S. Pat. No. 5,632,272, which is hereby incorporated by reference. In certain embodiments the ADC 2106 samples the signal into discrete voltage values and then converts the discrete sampled signal into a digital signal represented by digital values. In other embodiments, sampling and analog-to-digital conversion are performed by separate circuit components.

The digital signal then proceeds to a processor, such as a multi-purpose microprocessor, CPU, digital signal processor, or application specific integrated circuit (ASIC). In the depicted embodiment, the processor is a digital signal processor (DSP) 2108. The DSP 2108 processes the digital signal by implementing program code. The DSP 2108 in some embodiments uses the digital signal to determine or calculate a value of a physiological parameter of the patient. The DSP 2108 might also use the digital signal to calculate respiratory rate or heart rate according to an algorithm. Examples of such algorithms are described in International Application No. PCT/CA2005/000568, published as International Publication No. WO 2005/099562, and International Application No. PCT/CA2005/000536, published as International Publication No. WO 2005/096931, which are hereby incorporated by reference. In addition, the DSP 2108 may further provide a value or an indication of a physiological parameter to a display 2110 or to a storage device (not shown).

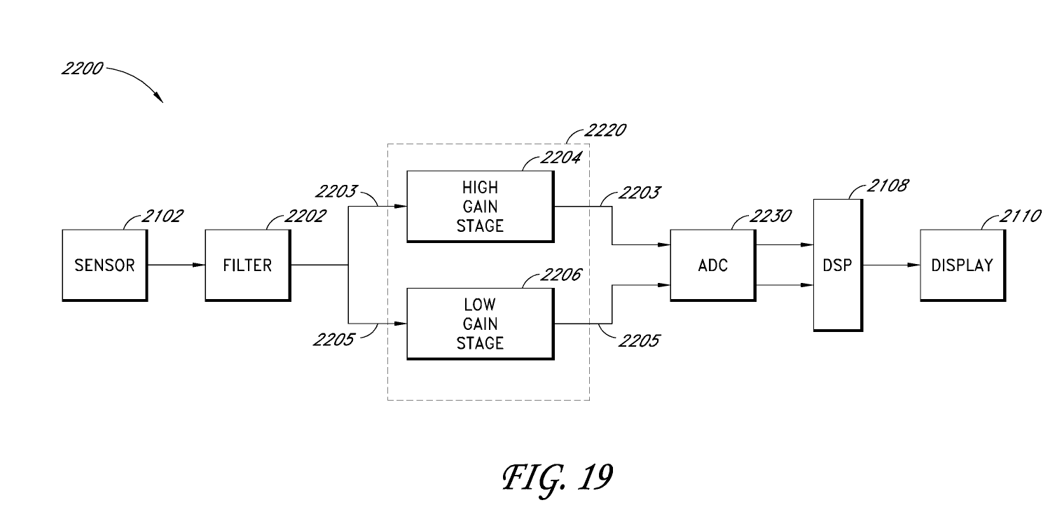

FIG. 19 depicts certain embodiments of an acoustic signal processing system 2200 in accordance with another embodiment of the present invention. In the acoustic signal processing system 2200, a sensor 2102 transmits a voltage signal to a filter 2202. The filter 2202 in certain embodiments modifies the voltage signal by, for example, smoothing or flattening the signal. In one embodiment, the filter 2202 is a high pass filter. The high pass filter allows high frequency components of the voltage signal above a certain predetermined cutoff frequency to be transmitted and attenuates low frequency components below the cutoff frequency. Low frequency signals are desirable to attenuate in certain embodiments because such signals can saturate amplifiers in the gain bank 2220.

Other types of filters may be included in the filter 2202. For example, the filter 2202 may include a low pass filter that attenuates high frequency signals. It may be desirable to reject high frequency signals because such signals often include noise. In certain embodiments, the filter 2202 includes both a low pass filter and a high pass filter. Alternatively, the filter 2202 may include a band-pass filter that simultaneously attenuates both low and high frequencies.

The output from the filter 2202 is split in certain embodiments into two channels, for example, first and second channels 2203, 2205. In other embodiments, more than two channels are used. For example, in some embodiments 3, 4, 8, 16, 32 channels or more are used. The voltage signal is transmitted on both first and second channels 2203, 2205 to gain bank 2220. Gain bank 2220 in certain embodiments includes one or more gain stages. In the depicted embodiment, there are two gain stages 2204, 2206. A high gain stage 2204 amplifies the voltage signal into a higher voltage signal. A low gain stage 2206 in certain embodiments does not amplify or attenuates the voltage signal. In alternative embodiments, the low gain stage 2206 may simply amplify the voltage signal with a lower gain than the gain in the high gain stage 2204.

The amplified signal at both first and second channels 2203, 2205 then passes to an analog-to-digital converter (ADC) 2230. The ADC 2230 has two input channels to receive the separate output of both the high gain stage 2204 and the low gain stage 2206. The ADC bank 2230 samples and converts analog voltage signals into digital signals. The digital signals then pass to the DSP 2108 and thereafter to the display 2110. In certain embodiments, a separate sampling module samples the analog voltage signal and sends the sampled signal to the ADC 2230 for conversion to digital form. Additionally, in certain embodiments two ADCs 2230 may be used in place of one ADC 2230.

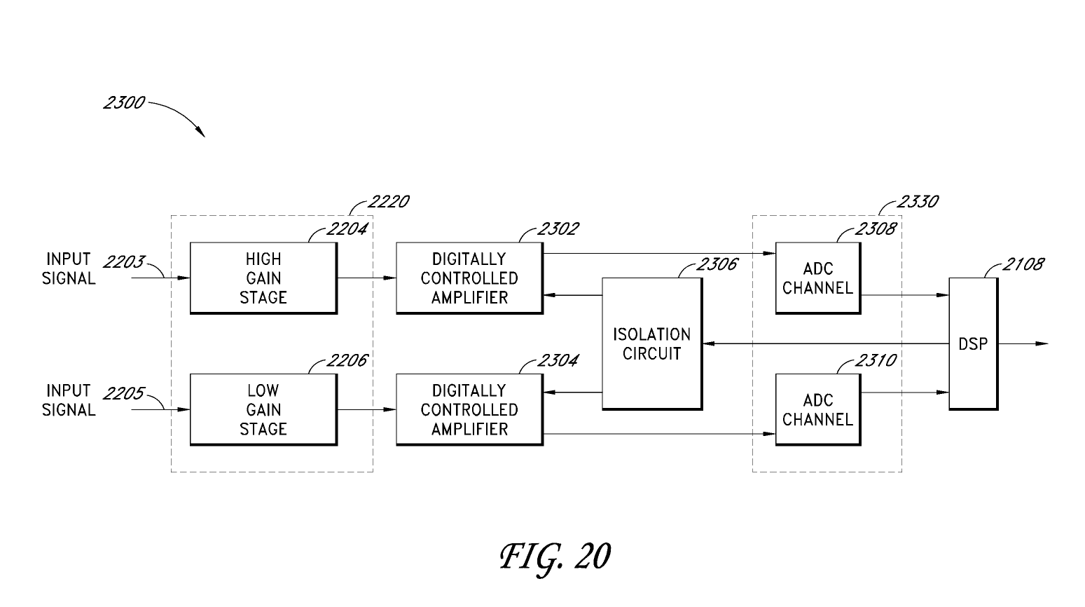

FIG. 20 depicts an acoustic signal processing system 2300 in accordance with yet another embodiment of the present invention. The system 2300 includes a gain bank 2220, which receives an input voltage signal from the sensor 2102 or the filter 2220 (not shown). Two channels 2203, 2205 transmit the voltage signal to the high gain stage 2204 and to the low gain stage 2206. The voltage signal then passes to digitally controlled amplifiers 2302 and 2304.

The digitally controlled amplifiers 2302, 2304 amplify the voltage signals received from the gain bank 2220. The gain level in the digitally controlled amplifiers 2302, 2304 is determined by a control signal from the DSP 2108. In certain embodiments, the DSP 2108 receives instructions from program code that indicate a desired gain level. The DSP 2108 then transmits the desired gain level to the digitally controlled amplifiers 2302, 2304, which in turn establish a gain level equal to or approximately equal to the desired gain level.

In certain embodiments, the DSP 2108 transmits control signals to the digitally controlled amplifiers 2302, 2304 via an isolation circuit 2306. The isolation circuit 2306 electrically isolates digital components such as the DSP 2108 from analog components such as the gain bank 2220. Isolating the digital components from the analog components protects the digital components from transient and potentially high voltages which could damage the digital components. In certain embodiments, the isolation circuit 2306 serves to protect the DSP 2108 from electrostatic discharge. The isolation circuit 2306 may also prevent the analog portion of the circuit from acting as a resistive load on the DSP 2108.

The isolation circuit 2306 in certain embodiments includes one or more DC to DC isolators, which may include two transformers (not shown). One transformer in some implementations is in communication with the DSP 2108, and the other transformer is in communication with the digitally controlled amplifiers 2302, 2304. When the DSP 2108 sends signals to the digitally controlled amplifiers 2302, 2304, changing magnetic fields in the transformer connected to the DSP 2108 induce magnetic fields in the transformer connected to the digitally controlled amplifiers 2302, 2304, thereby transmitting the signal from the DSP 2108 to the digitally controlled amplifiers 2302, 2304. The transformers are therefore inductively coupled and are not in direct electrical contact with one another. In alternative embodiments, the isolation circuit 2306 may include optoisolators or other forms of isolators in place of, or in addition to, transformers.

The output of the digitally controlled amplifiers 2302, 2304 proceeds to the ADC 2230. The output of the digitally controlled amplifier 2302 enters one channel 2308 of the ADC 2230, and the output of the digitally controlled amplifier 2304 enters another other channel 2310 of the ADC 2230. Using two channels 2308, 2310 in the same ADC 2230 synchronizes the voltage signal. In certain embodiments, synchronization of the two channels means that analog-to-digital conversion occurs at the same time or substantially the same time on each channel 2308, 2310. Thus, samples of the output of the digitally controlled amplifier 2302 correspond in time to samples of the output of the digitally controlled amplifier 2304. After sampling and conversion to digital form, the ADC 2230 passes two digital signals corresponding to the output from each digitally controlled amplifier 2302, 2304 to the DSP 2108.

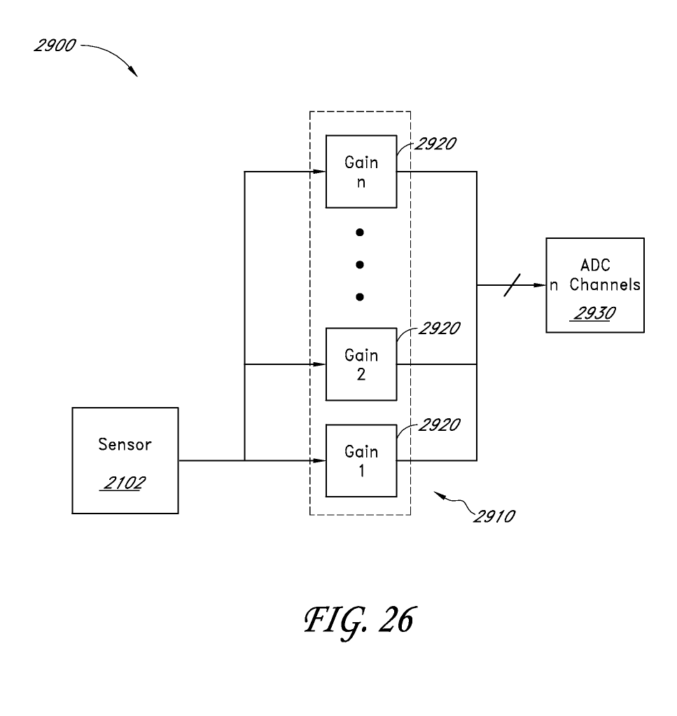

Other configurations of the ADC 2230 may be employed in various embodiments. For instance, two or more ADCs can be used in place of a single ADC 2230. Having two ADCs provides additional customizability, such as employing two ADCs with different resolutions (e.g., the number of discrete values that the ADC can produce over a range of voltage values). In addition, for a gain bank 2220 having more than two stages, as described more fully below in connection with FIG. 26, more than two ADCs may be employed. Alternatively, in embodiments where the gain bank 2220 has more than two stages, an ADC with more than two channels may be used. In still further embodiments, a combination of multi-channel and multi-ADC configuration may be employed.

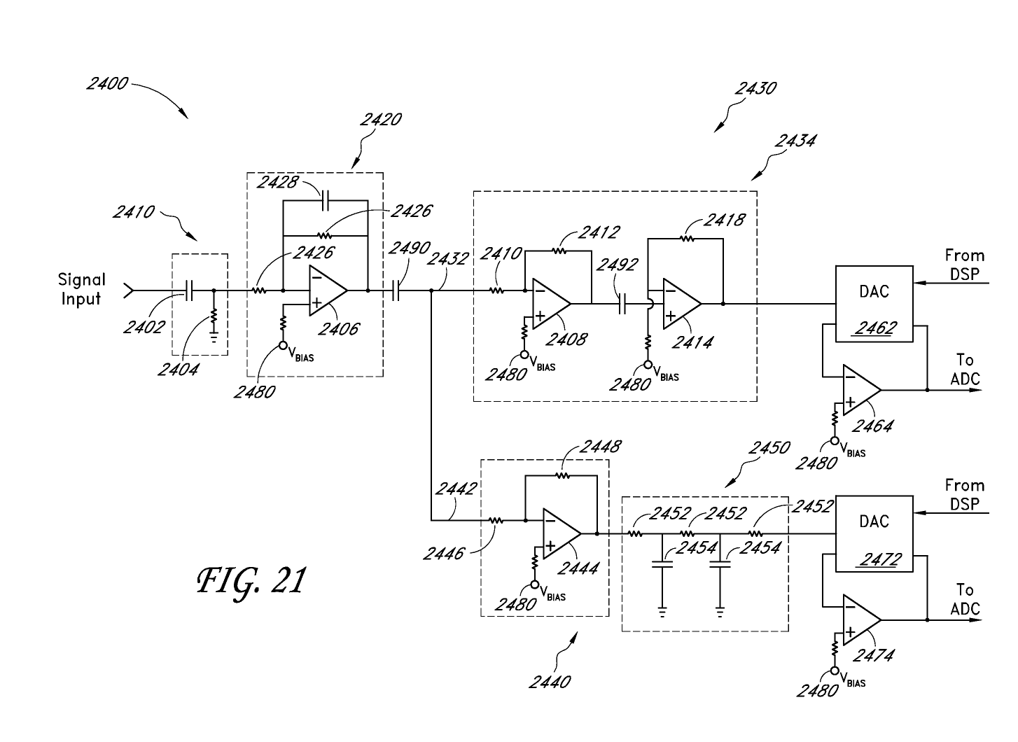

FIG. 21 illustrates embodiments of an acoustic signal processing system 2400. A high pass filter 2410 receives an input voltage signal from a sensor. The high pass filter includes a capacitor 2402 and a resistor 2404. In certain embodiments, the high pass filter 2410 attenuates signals of frequencies below a certain cutoff frequency. This cutoff frequency is determined by the values of the capacitor 2402 and of the resistor 2404. In one embodiment, the high pass filter attenuates signals that are below 100 Hertz (Hz). Signals below 100 Hz are attenuated because the sensors in certain embodiments are sensitive to low frequency sounds (e.g., below 100 Hz) and will saturate amplifiers in the acoustic signal processing system 2400. In other words, signals having frequencies below 100 Hz may create relatively high voltages in the sensor which may saturate the amplifiers in the acoustic signal processing system 2400.

A preprocessor stage 2420 receives the voltage signal from the high pass filter 2410. The preprocessor stage 2420 includes an operational amplifier (“op amp”) 2406, resistors 2426 and capacitors 2428. Like the high pass filter 2410, one or more resistors 2426 and capacitor 2428 determine a cutoff frequency. The preprocessor stage 2420 attenuates frequencies above the cutoff frequency. Attenuating frequencies above the cutoff frequency reduces the noise in the voltage signal, allowing for a more accurate analog-to-digital conversion of the signal.

The preprocessor stage 2420 in the depicted embodiment is also connected to a bias voltage source 2480. The bias voltage source 2480 creates a direct current (DC) bias in the acoustic signal processing system 2400. Without a bias voltage source 2480, the voltage signal output from the preprocessor stage 2420 would typically alternate in voltage about zero volts. In other words, part of the time the voltage signal may be above zero volts, and part of the time the voltage signal may be below zero volts. The bias voltage source 2480 adds a non-alternating voltage to the acoustic signal processing system 2400 which causes the voltage signal to alternate about the bias voltage instead of about zero volts. If the bias voltage source 2480 is high enough in voltage, all or substantially all of the voltage signal will output from the preprocessor stage 2420 at a level above zero volts.

In certain embodiments, a capacitor 2490 removes the DC component of the voltage signal so that DC current from the voltage source does not damage the sensor. Op amps 2408 and 2444, however, also receive the bias voltage source 2480, so that these op amps reintroduce the bias voltage into the voltage signal. Capacitor 2492 therefore also removes the DC component of the voltage signal, and op amp 2414 includes the bias voltage source 2480 in a similar manner.

From the preprocessor stage 2420, the voltage signal proceeds to two channels, namely channel 2432 and channel 2442. The two channels 2432, 2442 are connected to a gain bank 2430. The depicted gain bank 2430 includes a high gain stage 2434 and a low gain stage 2440. In certain embodiments, the high gain stage 2434 amplifies the voltage signal at a higher level than the low gain stage 2440 amplifies the voltage signal.

In one embodiment the high gain stage 2434 includes two op amps 2408, 2414. The op amp 2408 is connected to resistors 2410 and 2412, while the op amp 2414 is connected to resistors 2416 and 2418. The resistors 2410 and 2412 determine a gain value for the op amp 2408. Likewise, the resistors 2416 and 2418 determine a gain value for the op amp 2414. In the depicted embodiment, the op amp 2410 is in an inverting configuration. That is, the gain of the op amp 2410 is determined by the following equation:

Gain=−R_{f}/Rᵢ.

R_{f}, a feedback resistor, is divided by Rᵢ, an input resistor, and the negative value of this division is the gain of the op amp 2410. The negative sign indicates that the op amp 2410 inverts the phase of the voltage signal. In the depicted embodiment, the feedback resistor is the resistor 2412, and the input resistor is the resistor 2410. Conversely, the op amp 2414 is in the noninverting configuration, that is the gain of the op amp 2414 is determined by the following equation:

Gain=1+R_{f}/Rᵢ.

Here, the gain is 1 plus the division of the feedback resistor, which is the resistor 2418, by the input resistor, which is the resistor 2416. Because the gain in the op amp 2414 is positive, the op amp 2414 does not invert the phase of the voltage signal. In the depicted embodiment, the overall gain of the high gain stage 2434 is the sum of the absolute value (in dB) of the gain of each op amp 2408, 2414.

As one illustrative example, if the resistor 2410 is 158 kΩ (kilohms) and the resistor 2412 is 2 MΩ (megaohms), then the gain of the op amp 2408 is −2 MΩ/158 kΩ, which is 12.65. The gain can also be expressed in terms of decibels (dB), which is based on a logarithmic scale. The dB value of 12.65 is 20*log₁₀(12.65), which is 22.04 dB. Similarly, if the resistor 2416 has a value of 169 kΩ and the resistor 2418 has a value of 2 MΩ, the gain of the op amp is 12.83, and the gain in dB is 22.16. The combined gain of the high gain stage 2434 would then be the sum of the individual gain stages (in dB), or 22.04 dB plus 22.16 dB, which is 44.2 dB.

The low gain stage 2440 includes an op amp 2444 and resistors 2446 and 2448. The op amp 2444 is in the inverting configuration, and therefore has a gain value equal to the negative value of resistor 2448 divided by resistor 2416. Because the gain is negative, the op amp 2444 inverts the phase of the voltage signal. In the depicted embodiment it is advantageous to invert the voltage signal in the low gain stage 2440 because the op amp 2408 inverts the voltage signal in the high gain stage 2434. Consequently, the output signal of the high gain stage 2434 and low gain stage 2440 are at least partially in phase.

In alternative embodiments, where a 0 dB gain is desired on the low gain stage 2440, circuit components other than the op amp 2444 may be used. For instance, a resistor, wire, or other non-amplifying component may be used. However, in certain embodiments the op amp 2444 is employed despite its unity gain because the input impedance of the op amp 2444 is high. This high input impedance in certain embodiments reduces the current that is transmitted to the DAC 2472 and thereby protects the DAC 2472 from being damaged or destroyed by dangerously high currents. Similarly, the op amps 2408 and 2414 reduce the current that is transmitted to the DAC 2462.

The gain of the op amp 2444 is lower than the gain of the op amps 2408 and 2414. The low gain stage 2440 in certain embodiments has a gain of 1, or 0 decibels (dB), such that the low gain stage 2440 does not amplify the voltage signal. In certain embodiments, the high gain stage 2434 has a gain of 256, or 48.16 dB. The high gain stage 2434 therefore amplifies the input voltage signal approximately 256 times more than the low gain stage 2440 amplifies the signal. These gain values may be adjusted higher or lower to achieve a desired dynamic range. In addition, the high gain stage 2434 may have a gain of 1, and the low gain stage 2440 may have a gain of less than 1. Alternatively, the high gain stage 2434 may have a gain less than 1, and the low gain stage 2440 may have a gain that is much less than 1. Consequently, one of skill in the art will appreciate that several configurations of gain values may be employed in the acoustic signal processing system 2400.

In certain embodiments, the high gain stage 2434 may include only one op amp or possibly more than two op amps. In one embodiment, including only one op amp may reduce synchronization problems with the low gain stage 2440, as discussed more fully below. Likewise, additional op amps may reduce synchronization issues. Similarly, the low gain stage 2440 could include multiple op amps.

The op amps in the acoustic signal processing system 2400 may also be configured based on integrated circuit (IC) packaging. For instance, an IC having four op amps may be employed as a compact way to include the op amps 2406, 2408, 2414, and 2444 in the acoustic signal processing system 2400. Six, eight, or higher numbers of op amps included in one IC may be provided, as well as multiple ICs containing multiple op amps.

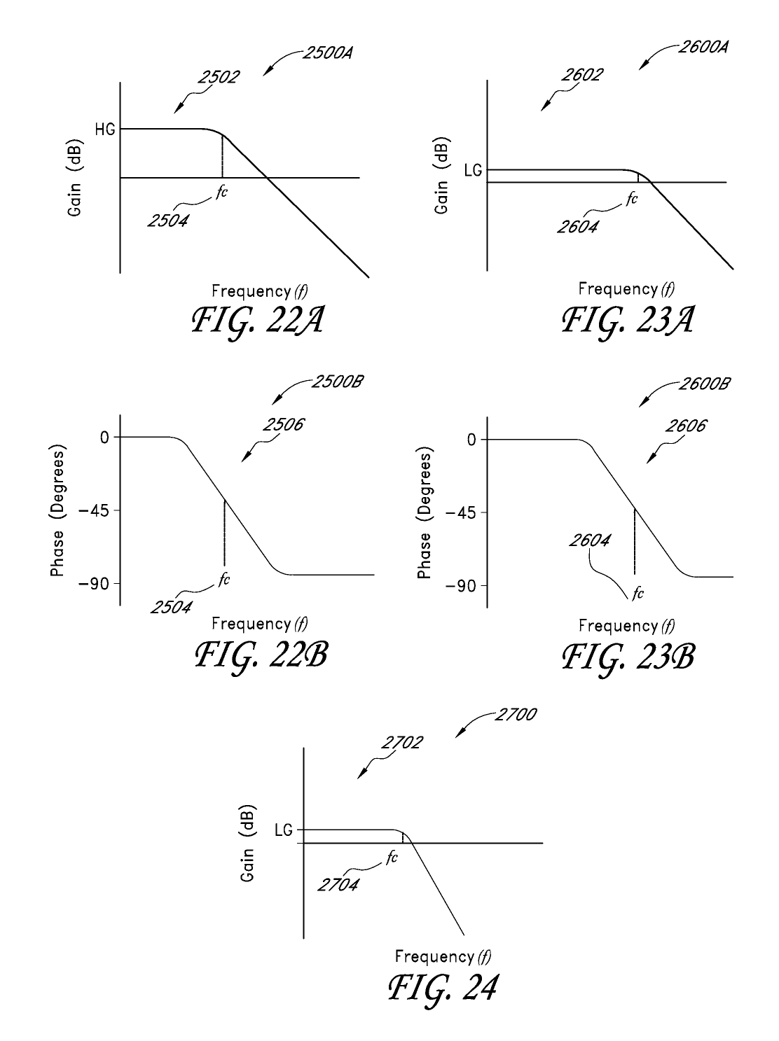

The output of the low gain stage 2440 is provided to a phase compensation circuit 2450, which adjusts the phase of the voltage signal output from the low gain stage 2440. The phase compensation circuit 2450 therefore compensates for phase differences between the voltage signal output from the high gain stage 2434 and the voltage signal output from the low gain stage 2440. In the depicted embodiment, the phase compensation circuit 2450 includes one or more resistors 2452 and one or more capacitors 2454. The phase compensation circuit 2450 therefore includes a low pass filter which changes the phase of the output voltage signal from the low gain stage 2440, as illustrated and described in greater detail with respect to FIGS. 22 through 24 below.

By compensating for phase differences, the phase compensation circuit 2450 ensures that the phase of the voltage signal output from the low gain stage 2440 is equal to or substantially equal to the phase of the voltage signal output from the high gain stage 2434. This is desirable in certain embodiments because a processor such as the DSP 2108 selects samples from both the high gain stage 2434 output and the low gain stage 2440 output. When the processor selects a signal from the low gain stage 2440 output, for example, this sample in certain embodiments should correspond in time with a sample from the high gain stage 2434. When the phases of each output signal match, the processor can construct a signal using samples from both output channels 2432, 2442, which correctly represents an amplified version of the input voltage signal over time.

In one embodiment, the phases of each voltage signal match perfectly, and in another embodiment, there is a slight phase delay. The phase delay can be within an accepted tolerance. For example, in one embodiment, the phase delay is five degrees. In certain embodiments, a slight phase delay between the two output signals is acceptable because it minimally distorts the signal constructed by the processor. Advantageously, the phase compensation circuit 2450 also maintains a constant but possibly large phase delay in certain embodiments so that the amount of permissible phase delay is limited only by the amount of memory reserved by the DSP 2108 to store or “buffer” the signals from each channel. The DSP 2108 compensates for the phase delay in one embodiment by selecting a sample on one channel 2432, 2442 that is shifted in time from a sample on the other channel. For example, if the symbol τ represents the amount of constant time delay caused by the constant phase delay, and if the symbol T_{S} represents the point in time a sample is received on one channel 2432, 2442, the DSP 2108 may obtain a sample from the other channel 2432, 2442 at time T_{S}+τ (or T_{S}−τ, in some implementations).

The phase delay might not be precisely known in some instances because tolerances of the resistors 2452 and the capacitors 2454 introduce uncertainty into the phase delay. For instance, precision resistors and capacitors often have tolerances of 1%, meaning that their stated value may vary plus or minus 1% of that value. In such instances, the precise phase delay may not be known with 100% accuracy. In addition, some types of capacitors (e.g., electrolytic capacitors) may dry out as they age, further increasing the tolerance and therefore the phase delay over time. Advantageously, software in the DSP 2108 may be configured to determine the phase delay by applying correlation, an indication of the relationship between two sets of data, between data from both the high gain channel 2432 and the low gain channel 2442. One embodiment of such correlation uses snapshots of data from times when the high gain channel 2432 is close to saturation. From this correlation, the DSP 2108 can precisely and dynamically estimate the exact phase delay.

In some embodiments, the phase compensation circuit 2450 compensates for differences in the gain bandwidth of the op amps 2408, 2414, and 2444. Gain bandwidth (GBW) in one implementation is the product of the gain and the cutoff frequency (e.g., the 3 dB bandwidth) of the op amp. For example, if the gain is 100 and the cutoff frequency is 1 kHz (1000 Hertz), the GBW of the op amp is 100 kHz. In other words, from a frequency range of 0 to approximately 1 kHz, the op amp amplifies the amplitude of an input signal by a factor of 100 (40 dB), but beyond 1 kHz, the gain attenuates quickly. The GBW in certain implementations is a constant value, so that a change in gain will effectuate a change in cutoff frequency. Thus, for an op amp with a GBW of 100 kHz, if the gain is reduced to 10 (20 dB), the cutoff frequency or bandwidth increases to 10 kHz.

Because the cutoff frequency can be changed by changing the gain, phase delays or mismatches may occur between two op amp stages having different gains. The high gain stage 2434 and low gain stage 2440 have different gains in certain embodiments, and if op amps 2408, 2414, and 2444 have similar GBW values, the phase output of each op amp will therefore be different from the phase output of the other op amps. The phase compensation circuit 2450 in certain embodiments therefore matches or substantially matches the bandwidth of the low gain stage 2440 to the bandwidth of the high gain stage 2434. Because the bandwidths of each stage 2434, 2440 are equal or substantially equal, the phases of the voltage signals outputs from each stage are equal or substantially equal, as depicted more fully in FIGS. 22 through 24 below. In addition, phase compensation circuit 2450 may also be placed before the low gain stage 2440 in certain embodiments.

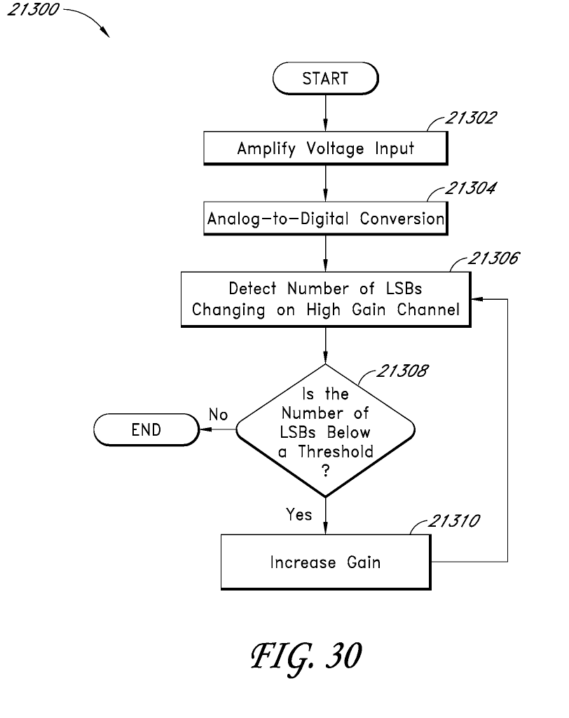

From the high gain stage 2434, the voltage signal on channel 2432 proceeds to the digital to analog converter (DAC) 2462. Likewise, from the phase compensation circuit 2450, the voltage signal on channel 2442 proceeds to the DAC 2472. The DAC 2462 and the DAC 2472 in conjunction with op amps 2464 and 2474, in certain embodiments act as digitally controlled amplifiers. In particular, the DACs 2462, 2472 act as digital potentiometers, receiving a digital input from a DSP and changing one or more internal resistance values in response to receiving the digital input. These resistance values determine a gain value for each of the op amps 2464, 2474. This gain value may be equivalent or substantially equivalent for each op amp 2464, 2474. In certain embodiments, the gain value is used to calibrate the acoustic signal processing system 2400, as described more fully in connection with FIG. 30, below.

The DACs 2462, 2472 in certain embodiments are controlled synchronously by the DSP 2108 through a single isolation circuit, such as the isolation circuit 2306 depicted in FIG. 20. Because the DACs 2462, 2472 are synchronized, the output signals from op amp 2464 and op amp 2474 remain in phase or substantially in phase as they are transmitted to an ADC (not shown). In one embodiment, the output signals are perfectly in phase. In alternative embodiments, they are slightly out of phase, such as by 5 degrees, 10 degrees, or some other small amount. The DACs 2462, 2472 therefore in these embodiments maintain a constant delay between the output signals, so as to minimize distortion in later signal construction by the processor. Moreover, while two DACs 2462, 2472 are depicted, one DAC with two-channels may be used instead to achieve greater synchronization. Likewise, with more than two gain stages, more than two DACs may be used, or one DAC with more than two channels may be used.

While DACs are one embodiment of potentiometer, in alternative embodiments analog potentiometers may be employed. In such instances, the analog potentiometer does not receive input from a processor, but is instead actuated by another circuit component or by a technician.

In the depicted embodiment, capacitors are not placed after the op amp 2414 and the op amp 2444 (except for the capacitors 2454, which are described below). The output voltage signal of the op amp 2414 and the op amp 2444 therefore retain their DC components. In addition, the op amps 2464 and 2474 have bias voltage sources 2480. The acoustic signal processing system 2400 therefore sends a positive voltage signal to an ADC. The ADC of certain embodiments therefore does not determine negative digital values, which reduces the complexity of the output signal from the ADC. For example, some implementations of ADCs use a special encoding scheme such as “two's complement” to determine negative numbers, and eliminating this scheme may reduce the complexity of the ADC.

FIGS. 22A and 22B depict embodiments of amplitude and phase plots of an op amp in a high gain stage, such as the high gain stage 2434 of FIG. 21. FIG. 22A depicts an amplitude plot 2500A corresponding to a high gain stage op amp. The amplitude plot 2500A depicts values of gain corresponding to values of frequency. For low frequencies, the gain level 2502 is at a high level (HG). However, beginning approximately at a cutoff frequency 2504, the gain level 2502 decreases steadily, indicating that the op amp attenuates signals above the cutoff frequency. Because signals above the cutoff frequency 2504 are attenuated, the cutoff frequency 2504 is equivalent to the bandwidth of the op amp.

FIG. 22B depicts a phase plot 2500B corresponding to the amplitude plot 2500A. The phase plot 2500B graphically depicts the phase output of signals according to frequency. The phase plot 2500B indicates that the op amp changes the phase of a signal passing through the op amp at certain frequencies. As the frequency of the input signal exceeds the cutoff frequency 2504, the phase approaches a change of 90 degrees.

FIG. 23A depicts embodiments of amplitude and phase plots of an op amp in a low gain stage, such as the low gain stage 2440 of FIG. 21. The gain level 2602 (LG) of the low gain op amp is significantly lower than the gain level 2502 of the high gain op amp. In contrast, the bandwidth of the low gain op amp as indicated by the cutoff frequency 2604 is significantly higher than the high gain op amp's bandwidth, which has a much lower cutoff frequency 2504. This difference in bandwidth results from the GBW effect. FIGS. 2500A and 2600A therefore illustrate that in an op amp with a higher level of gain, the bandwidth is less than in an op amp with a lower level of gain, assuming that both op amps have the same GBW.

FIG. 23B depicts a phase plot 2600B corresponding to the amplitude plot 2600A. The phase plot 2600B graphically depicts the phase output of signals according to frequency. The phase plot 2600B indicates that the low gain op amp changes the phase of a signal passing through the op amp at certain frequencies. As the frequency of the input signal exceeds the cutoff frequency 2604, the phase approaches a change of 90 degrees. Because the cutoff frequency 2604 of the low gain op amp is higher than the cutoff frequency 2504 of the high gain op amp, the phase change in each op amp occurs at different frequencies. Consequently, the same signal output from each op amp may differ in phase.

FIG. 24 depicts an amplitude plot 2700 of a low pass filter, such as the low pass filter in the phase compensation circuit 2450. The amplitude plot 2700 has the same gain level 2702 (LG) as the low gain op amp in amplitude plot 2600A and the same bandwidth as the bandwidth of the high gain op amp. In other words, cutoff frequency 2704 is approximately equivalent to cutoff frequency 2504. In certain embodiments, the low pass filter restricts the bandwidth of signals outputting from the low gain op amp to the low pass filter cutoff frequency 2604. By virtue of this filtering, the low pass filter reduces the overall bandwidth of the low gain stage to match the bandwidth of the high gain stage. Consequently, the phase of the low gain stage with an added low pass filter is the same or approximately the same as the phase of the high gain stage.

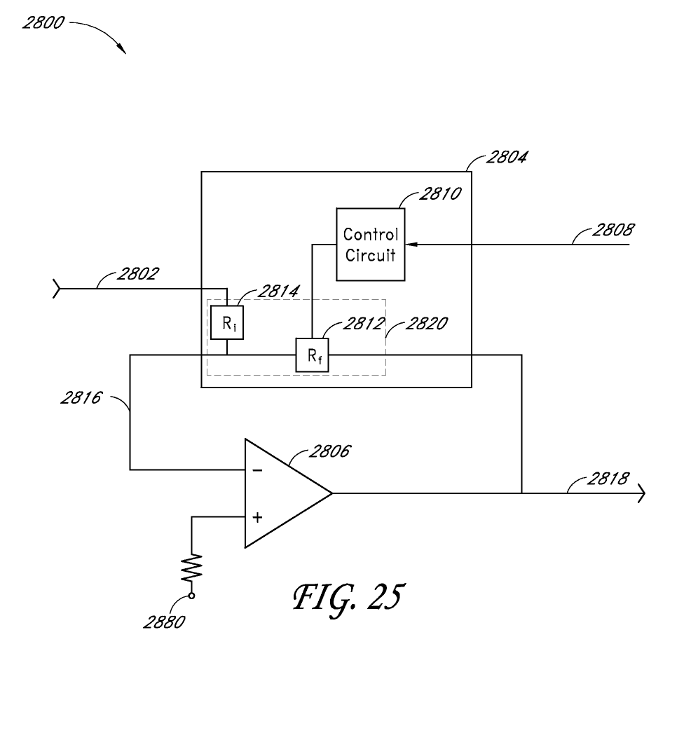

FIG. 25 depicts certain embodiments of a digitally-controlled amplifier 2800. The digitally-controlled amplifier 2800 includes a digitally-controlled potentiometer 2804. In certain embodiments, the digitally-controlled potentiometer 2804 is a DAC, such as one of the DACs 2464, 2474. In alternative embodiments, the digitally-controlled potentiometer 2804 is a digital potentiometer integrated circuit. In embodiments where a DAC is employed, the DAC may be a delta-sigma converter, such as the delta-sigma converter described in U.S. Pat. No. 5,632,272, which is hereby incorporated by reference in its entirety.

The digital potentiometer 2804 receives a digital control signal 2808. Using information obtained from the digital control signal 2808, a control circuit 2810 sets resistor values in a resistor network 2820. The control circuit 2810 in one implementation includes a multiplexer (MUX), and the resistor network 2820 includes a resistor ladder. In one embodiment, the resistor network 2820 is configured to provide a feedback resistor (R_{f}) value 2812 and an input resistor value 2814. In certain embodiments, the feedback resistor value 2812 is a feedback resistor for an op amp 2806, and the input resistor value 2814 is the input resistor for the op amp 2806. In alternative embodiments, the resistor network 2820 provides either the feedback resistor value 2812 or the input resistor value 2814, but not both.

A gain value for the op amp can be established by setting the resistor values 2812 and 2814 of the resistor network 2820 to a desired level. The gain value of the op amp 2806 shown in the inverting configuration is equal to the negative value of the feedback resistor value 2812 divided by the input resistor value 2814. While the inverting configuration of the op amp 2806 is shown, the noninverting configuration or other configurations may also be used. With this feedback network of resistors, the op amp 2806 can amplify input signals at a desired gain value determined by the digitally-controlled potentiometer 2804 and transmit the amplified signals as a voltage output at 2818.

FIG. 26 depicts embodiments of a gain bank 2910 in accordance with yet another embodiment. The gain bank 2910 includes multiple gain stages 2920 which each receive a voltage signal input from a sensor. In certain embodiments, each gain stage 2920 has a different gain level from the other gain stages 2920. Including multiple gain stages 2920 in the gain bank 2910 generates a larger dynamic range, or amplification range, in certain embodiments than gain banks 2910 with fewer gain stages 2920. In addition, in certain embodiments the gain stages 2920 output an amplified voltage signal that is processed in a single ADC 2930. By processing the output of each gain stage 2920 in a single ADC 2930, the output signal of each gain stage 2920 may be synchronized or substantially synchronized.

Various embodiments of the gain bank 2910 may include two, three, or more gain stages 2920. The gain values of each gain stage 2920 may be different, or two or more gain stages 2920 may have equivalent values. In addition, some gain stages 2920 may attenuate the input voltage signal, others may have neither amplify nor attenuate (e.g., have 0 dB gain), and still others may amplify the signal. One of skill in the art will appreciate that many combinations of gain stages 2920 may be derived to attain a desired dynamic range. Moreover, the gain bank 2910 may be used with any of the acoustic signal processing systems described above, such as the acoustic signal processing systems 2100, 2200, 2300, or 2400.

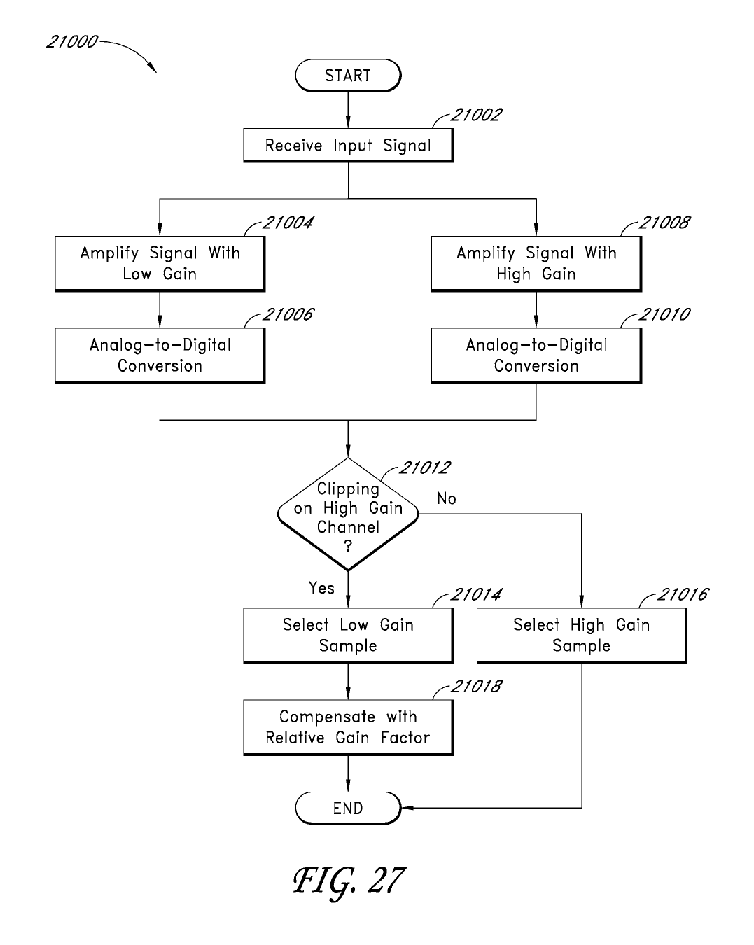

FIG. 27 depicts a method 21000 for automatically adjusting the gain of an input signal in accordance with certain embodiments of the present invention. The method 21000 may be performed by any of the acoustic signal processing systems described above. In addition, the method 21000 may be performed on a sample-by-sample basis such that no samples are “lost” due to amplifier saturation or lack of synchronization introduced by GBW differences. Thus, parallel processing of an input signal may be achieved in a system with multiple gain stages.

At 21002 an input signal is received. The input signal is amplified with a low gain at 21004 and is amplified with a high gain at 21008. At 21006 and 21010, the low gain signal and the high gain signal are each converted from analog to digital form, where each signal includes one or more digital samples.

At 21012, it is determined whether a digital sample is clipping on the high gain channel. Clipping occurs when an amplifier saturates and is often seen on an oscilloscope or display as a flat line instead of a changing waveform. In certain embodiments, clipping indicates that amplifying the signal with a high gain at 21008 has saturated one or more high gain amplifiers. If a sample is clipped in the high gain amplifiers, then the method 21000 selects the digital sample on the low gain channel at 21014. However, if there is no clipping on the high gain channel, the processor selects the digital sample on the high gain channel at 21018, and the method ends.

At 21016, the processor compensates for the low level of the low-gain digital sample by multiplying the digital sample with a relative gain factor. In certain embodiments, this relative gain factor is the ratio of the gain on the high gain channel to the gain on the low gain channel. For example, in one embodiment if the difference in gain between the high gain and low gain channels is 256, the relative gain factor might be 256.

The processor in various embodiments multiplies samples in real time. In other embodiments, the processor constructs a data structure containing samples from the high and low gain channels and then multiplies certain low gain samples in the data structure by a compensation factor. After compensation occurs at 21018, the method 21000 ends. Method 21000 therefore accomplishes an automatic, rapid gain adjustment without relying on human intervention to adjust an analog gain value.

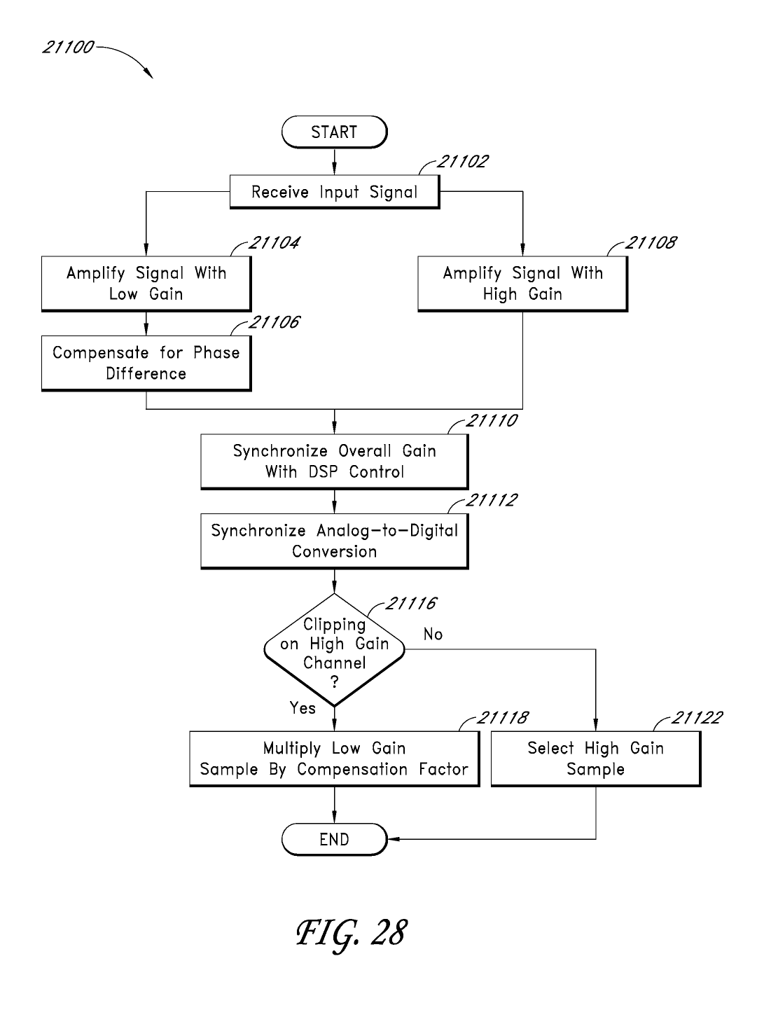

FIG. 28 depicts certain embodiments of a method 21100 for constructing an output signal from synchronized-phase input signals. The method 21100 may be performed by any of the acoustic signal processing systems described above. In addition, the method 21000 may be performed on a sample-by-sample basis such that no samples are “lost” due to amplifier saturation or lack of synchronization, including asynchronization introduced by GBW differences. Thus, parallel processing of an input signal may be achieved in a system with multiple gain stages.

At 21102 an input signal is received from a sensor, such as any sensor 2102 discussed above. The signal is amplified with low gain at 21104 and with high gain at 21108. At 21106 the amplified signal is compensated for possible phase differences by using a phase compensation circuit in a manner similar to that described above.

At 21110 an overall gain is achieved and synchronized by processor control. In certain embodiments, the overall gain increases the gain of both a high gain and a low gain channel, such as by using a DAC in communication with an isolation circuit and a DSP. This overall gain value may be used for calibration purposes, such as described in connection with FIG. 30, below.

A synchronized analog-to-digital conversion takes place at 21112. This synchronization occurs in certain embodiments when a single, multi-channel ADC receives inputs from both low and high gain channels. A synchronized analog to digital conversion prevents further delays from occurring in the acoustic monitoring system.

At 21116 it is determined whether there is clipping on the high gain channel. Clipping may be determined by comparing the value of one or more samples to a maximum or saturated value. If there is clipping on the high gain channel, then at 21118 the method 21100 multiplies the low gain channel sample by a compensation factor. The processor in various embodiments multiplies samples in real time. In other embodiments, the processor constructs a data structure containing samples from the high and low gain channels and then multiplies certain low gain samples in the data structure by a compensation factor. However, if there is no clipping in the high gain channel, then the high gain sample is selected instead and the method ends. The method 21100 therefore constructs an output signal with minimal distortion by synchronizing the phases of the input signals, such as is depicted in FIGS. 24A through 24F above.

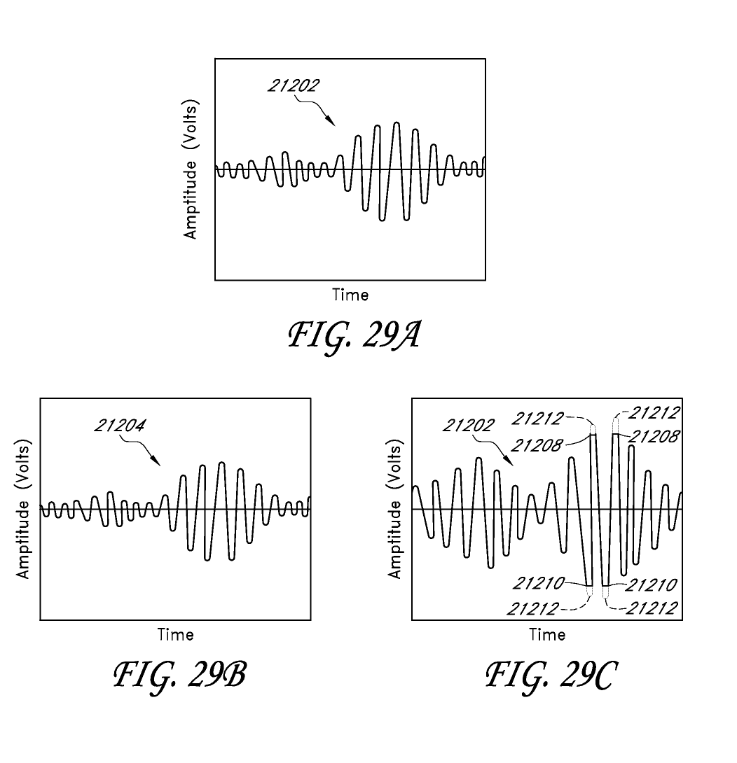

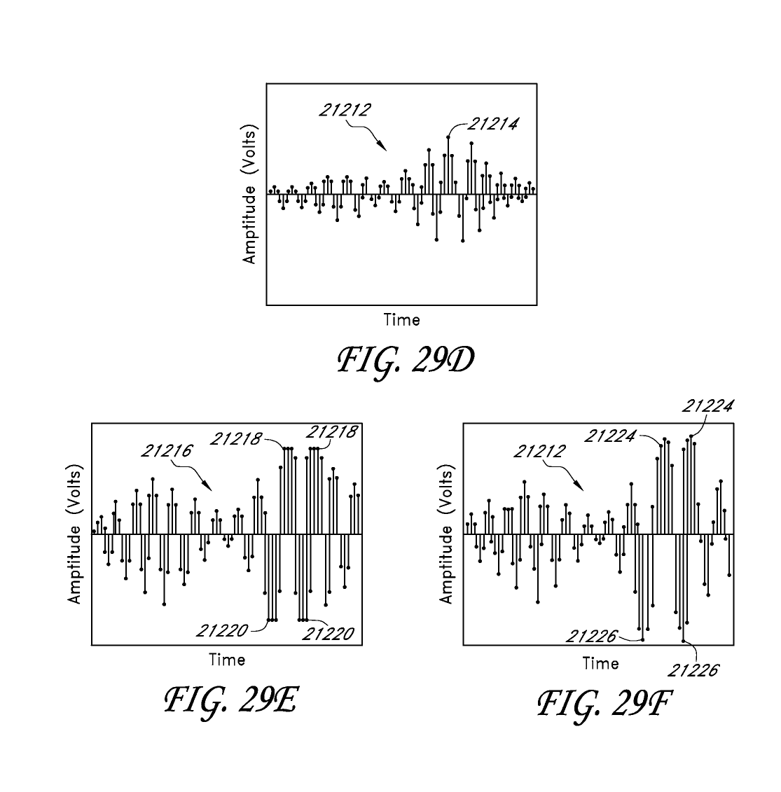

FIG. 29A through 29F depict certain embodiments of an implementation of the methods 2900 or 21100 and of one or more acoustic signal processing systems described above. FIG. 29A depicts an analog input signal 21202 that provided by a sensor. The amplitude of the analog input signal 21202 varies in time according to breathing and other sounds from a patient.

FIG. 29B depicts a low gain amplified signal 21204, which corresponds to the analog input signal 21202. The low gain amplified signal 21204 is a low gain amplified version of the analog input signal 21202. In the depicted embodiment, the low gain amplified signal 21204 has the same or substantially the same amplitude as the analog input signal 21202, indicating that little or no amplification has occurred. In other embodiments, the amplified signal 21204 may be greater or lesser in amplitude than the analog input signal 21202.

FIG. 29C depicts a high gain amplified version of the analog input signal 21202. The amplitude of the high gain amplified signal 21206 is greater than the amplitude of the low gain amplified signal 21204 at corresponding times. In the depicted embodiment, the high gain amplified signal 21206 includes clipped portions 21208 and 21210. The clipped portions appear as flat lines, indicating that a high gain amplifier stage saturated at these portions 21208, 21210 of the high gain amplified signal 21206. In other words, the high gain amplifier stage could not amplify the amplified analog signal 21202 to any higher value because the gain of the op amp was limited by physical constraints in its circuitry. The phantom lines 21212 indicate where the high gain amplified signal 21206 would have been had the high gain amplifier stage not saturated.

FIG. 29D shows a digitally sampled version of the low gain amplified signal 21204. The digital signal 21212 is a sampled version of the low gain amplified signal 21204, and samples 21214 indicate discrete points where the low gain amplified signal 21204 has been sampled. For clarity, discrete points are shown rather then voltage levels. However, in certain embodiments, a zero order or higher-order hold may be employed.

FIG. 29E shows a digitally sampled version of the high gain amplified signal 21206. The digital signal 21216 is a sampled version of the high gain amplified signal 21206. The digital signal 21216 also has clipped samples 21218 and 21220 corresponding to the clipped portions 21208 and 21210 of FIG. 29C.

FIG. 29F illustrates a digital signal 21222 constructed by a processor executing the method 2600 or the method 2800, below. The digital signal 21222 is constructed by first determining whether a sample from the high gain channel is clipping. If the sample is not clipping, a processor selects the sample. If the sample is clipping, the processor selects a sample from the low gain channel that corresponds in time to the sample from the high gain channel.

If the processor selects the low gain sample, then the processor also multiplies the low gain sample by a relative gain factor. The multiplied sample is therefore equivalent or substantially equivalent to the value that the clipped sample would have had, had the sample not clipped. The method 2600 executes for each sample until a signal is constructed, such as the digital signal 21222 in FIG. 29F. In the digital signal 21222, multiplied values 21224, 21226 illustrate construction of the proper values (represented by the phantom lines 21212) in place of clipped values 21208, 21210.

In alternative embodiments, the high gain sample may be divided by a relative gain factor (e.g., 256) when no clipping occurs, and the low gain sample may be used when the corresponding high gain sample clips. Other arrangements, including multiple gain stages such as depicted in FIG. 26, may further select samples in different ways. For instance, if three gain stages are employed, samples may be primarily selected from one gain stage. If the output signal saturates, a lower gain bank output sample might be selected, and if the output signal is too weak, with insufficient resolution, a higher gain bank output sample might be selected. Other combinations of gain banks and sample selecting processes may be determined as will be readily understood by one of ordinary skill in the art.

FIGS. 29A through 29F illustrate that in certain embodiments, synchronization of the phase of the low gain signal and the high gain signal aids in construction of the proper digital signal. If the low gain and high gain signals were out of phase, an improper low gain sample might be selected, causing distortion in the output signal.

FIG. 30 depicts embodiments of a method 21300 for calibrating an acoustic signal processing system using a digitally controlled amplifier. The method 21300 may be performed by any of the acoustic signal processing systems described above. In addition, the method 2600 may be performed on a sample-by-sample basis such that no samples are “lost” due to saturation of amplifiers or to lack of synchronization introduced by GBW differences. Thus, parallel processing of an input signal may be achieved in a system with multiple gain stages.

At 21302, a voltage input from a sensor is amplified by a gain bank. The gain bank may include a low gain stage and a high gain stage such as any of those discussed above. The amplified signal from both the low gain stage and the high gain stage is converted from analog to digital form at 21304. Each digital signal includes samples which are represented by binary bits of data. The number of least significant bits (LSBs) that changes in value from sample to sample of the high gain signal is detected at 21306. If the number of changing LSBs is below a threshold value at 21308, the gain of a digitally controlled amplifier is increased at 21310. If, however, the gain is not below the threshold at 21308, the method ends.

In alternative embodiments, if the number of changing LSBs is greater than an upper threshold value, gain is reduced. Moreover, gain may be adjusted as needed to maintain the gain between upper and lower threshold values. By changing the gain of a digitally controlled amplifier when the gain of the amplifier outputs values above or below a threshold, the method 21300 self-calibrates in response to receiving input signals. Self-calibration or automatic adjustment in this manner replaces the need for a nurse or other technician to calibrate an acoustic signal processing system. In addition, the method 21300 can be performed at initial system calibration and/or during operation of the acoustic signal processing system to ensure that “baseline” gain is appropriately, automatically set.

Any of the above-described physiological monitoring systems may be implemented using a variety of types of sensors. A variety of sensor embodiments, suitable for use with any of the systems described herein, will now be disclosed. In some cases, the sensor is configured to sense more than one biological or physiological parameter.

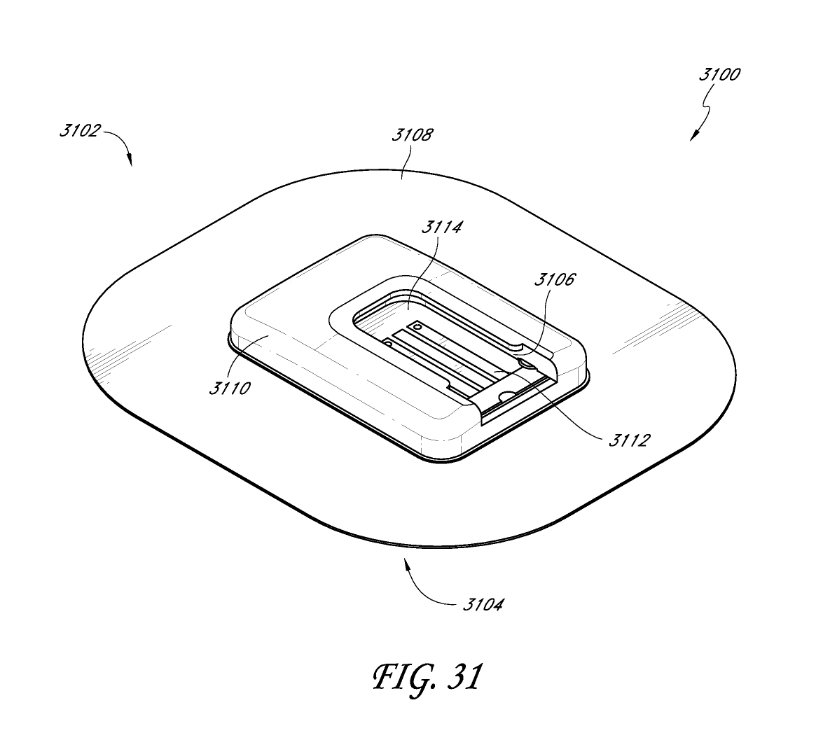

FIG. 31 illustrates a top perspective view of a multi-parameter sensor assembly 3100 in accordance with one embodiment of the present invention. The multi-parameter sensor assembly 3100 includes a cap sub-assembly 3102 and a sensor sub-assembly 3104. When coupled to one another as shown, the interface of the cap sub-assembly 3102 and sensor sub-assembly 3104 create a slot 3106 into which a connector of a sensor cable (not shown) may be removably attached.

The cap sub-assembly 3102 includes a patient adhesive 3108 (e.g., in some embodiments, tape, glue, a suction device, etc.) attached to a cap 3110. The patient adhesive 3108 has an adhesive surface that can be used to secure the multi-parameter sensor assembly 3100 to a patient's skin. A removable backing is provided with the patient adhesive 3108 to protect the adhesive surface prior to affixing to a patient's skin.

When the sensor cable is attached to the multi-parameter sensor assembly 3100, sensor cable contacts are placed in electrical contact with contract strips 3112 of a printed circuit board 3114. Through this contact, electrical signals are communicated from the multi-parameter sensor assembly 3100 to a physiological monitor, as discussed in greater detail below. Additional aspects of the printed circuit board 3114 are provided in greater detail below as well.

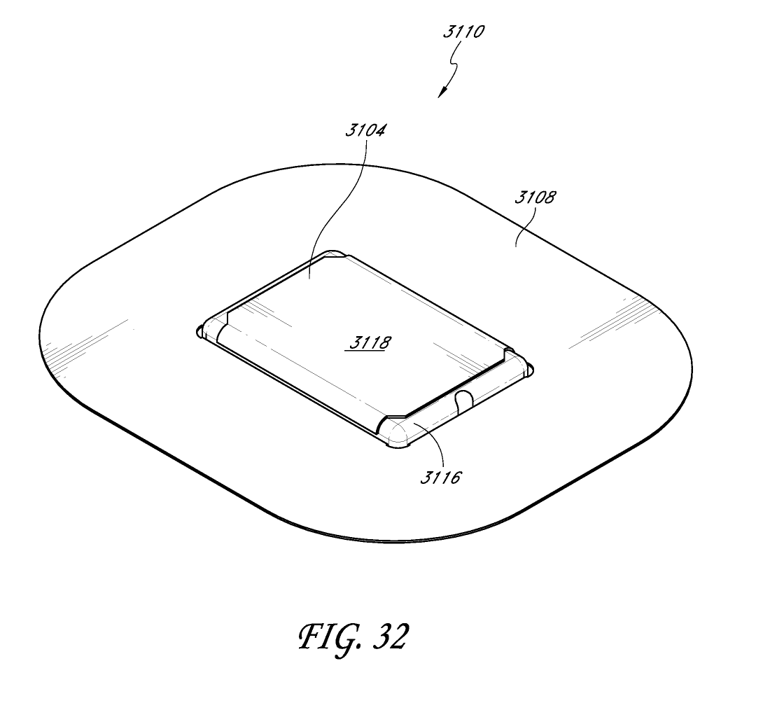

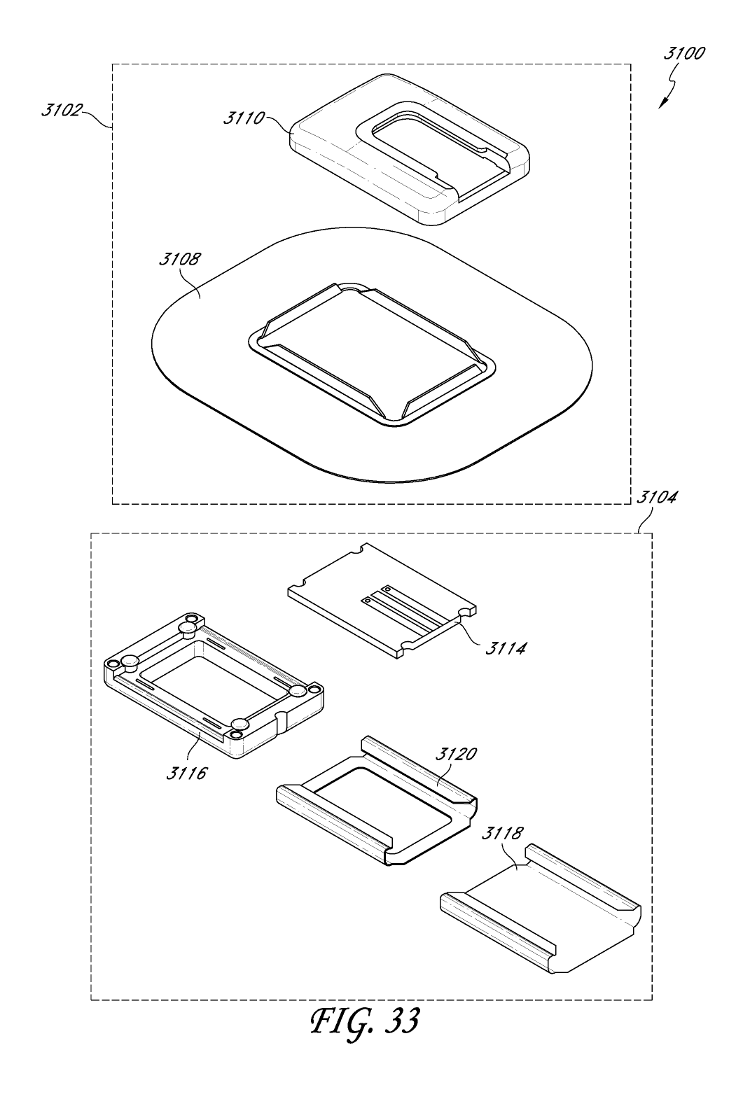

FIG. 32 illustrates a bottom perspective view of the multi-parameter sensor assembly 3100 of FIG. 31. The adhesive surface of the patient adhesive 3108 surrounds the sensor sub-assembly 3104. The sensor sub-assembly 3104 includes a frame 3116, which supports a sensing element 3118. The sensor sub-assembly 3104 also includes a bonding layer 3120 and the printed circuit board 3114, as can be seen in more detail in FIGS. 33, 34, and 40.

In one embodiment, the sensing element 3118 is a piezoelectric film, such as described in U.S. Pat. No. 6,661,161, incorporated by reference herein. In some embodiments, the sensing element 3118 includes one or more of crystals of tourmaline, quartz, topaz, cane sugar, and/or Rochelle salt (sodium potassium tartrate tetrahydrate). In other embodiments, the sensing element 3118 includes quartz analogue crystals, such as berlinite (AlPO₄) or gallium orthophosphate (GaPO₄), or ceramics with perovskite or tungsten-bronze structures (BaTiO₃, SrTiO₃, Pb(ZrTi)O₃, KNbO₃, LiNbO₃, LiTaO₃, BiFeO₃, NaₓWO₃, Ba₂NaNb₅O₅, Pb₂KNb₅O₁₅).

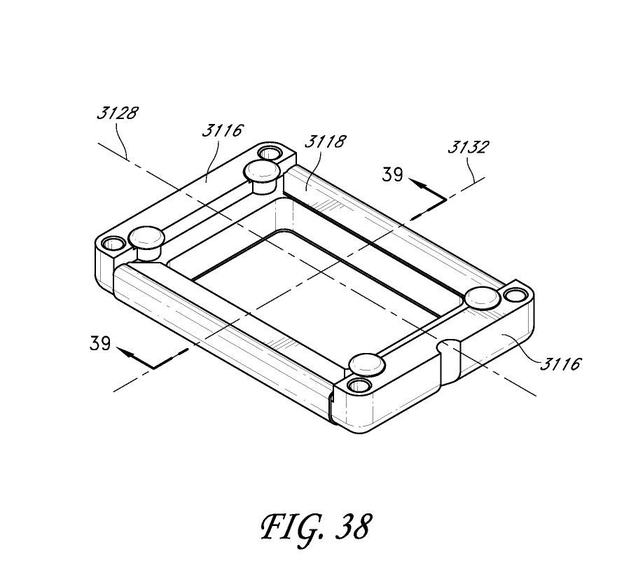

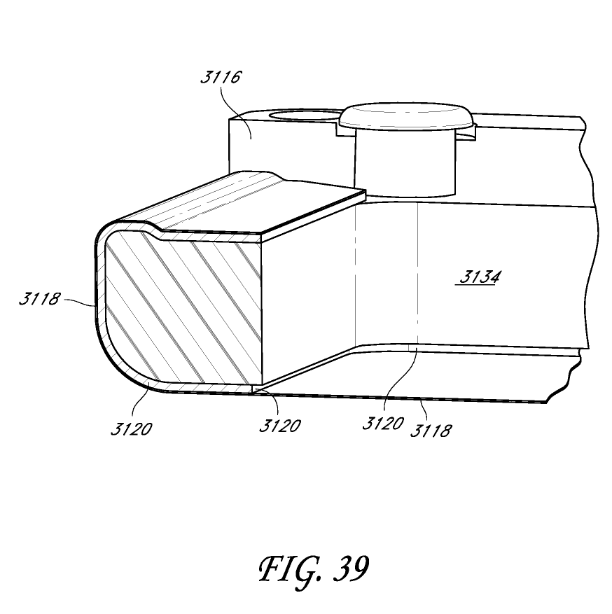

In other embodiments, the sensing element 3118 is made from a polyvinylidene fluoride plastic film, which develops piezoelectric properties by stretching the plastic while placed under a high pooling voltage. Stretching causes the film to polarize and the molecular structure of the plastic to align. For example, stretching the film under or within an electric field causes polarization of the material's molecules into alignment with the field. A thin layer of conductive metal, such as nickel-copper or silver is deposited on each side of the film as electrode coatings. The electrode coating provides an electrical interface between the film and a circuit. Additional details regarding the sensing element 3118 are provided with respect to FIGS. 38 and 39.

In operation, the piezoelectric material becomes temporarily polarized when subjected to a mechanical stress, such as a vibration from an acoustic source. The direction and magnitude of the polarization depend upon the direction and magnitude of the mechanical stress with respect to the piezoelectric material. The piezoelectric material will produce a voltage and current, or will modify the magnitude of a current flowing through it, in response to a change in the mechanical stress applied to it. In one embodiment, the electrical charge generated by the piezoelectric material is proportional to the change in mechanical stress of the piezoelectric material.

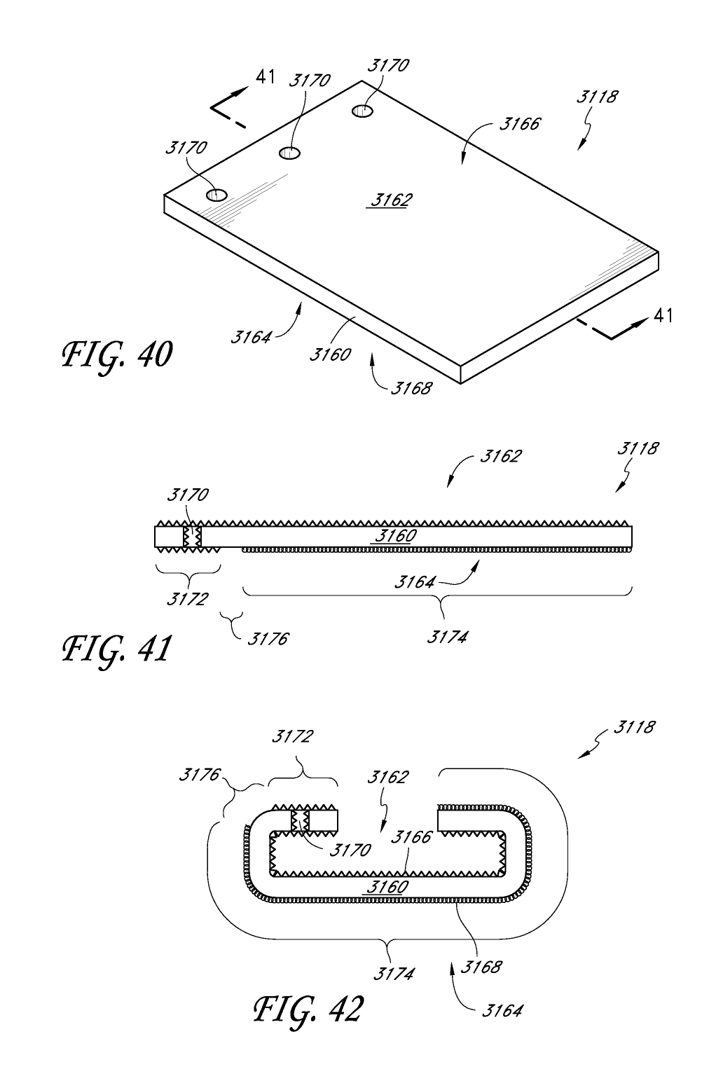

Piezoelectric material generally includes first and second electrode coatings applied to the two opposite faces of the material. The voltage and/or current through the piezoelectric material are measured across the first and second electrode coatings, as described in greater detail below with respect to FIGS. 40-42. Therefore, stresses produced by acoustic waves in the piezoelectric material will produce a corresponding electric signal. Detection of this electric signal is generally performed by electrically coupling the first and second electrode coatings to a detector circuit. In one embodiment, a detector circuit is provided with the printed circuit board 3114, as described in greater detail below.

By selecting the piezoelectric material's properties and geometries, a sensor having a particular frequency response and sensitivity can be provided. For example, the piezoelectric material's substrate and coatings, which generally act as a dielectric between two electrodes, can be selected to have a particular stiffness, geometry, thickness, width, length, dielectric strength, and/or conductance. For example, in some cases stiffer materials, such as gold, are used as the electrode. In other cases, less stiff materials, such as silver, are employed. Materials having different stiffness can be selectively used to provide control over sensor sensitivity and/or frequency response.

The piezoelectric material, or film, can be attached to, or wrapped around, a support structure, such as a frame. The geometry of the piezoelectric material can be selected to match the geometry of the frame. Overall, the sensor is optimized to pick up, or respond to, a particular desired sound frequency, and not other. The frequency of interest generally corresponds to a physiological condition or event that the sensor is intended to detect, such as internal bodily sounds, including, cardiac sounds (e.g., heart beats, valves opening and closing, fluid flow, fluid turbulence, etc.), respiratory sounds (e.g., breathing, inhalation, exhalation, wheezing, snoring, apnea events, coughing, choking, water in the lungs, etc.), or other bodily sounds (e.g., swallowing, digestive sounds, gas, muscle contraction, joint movement, bone and/or cartilage movement, muscle twitches, gastro-intestinal sounds, condition of bone and/or cartilage, etc.).

The surface area, geometry (e.g., shape), and thickness of the piezoelectric material generally defines a capacitance. The capacitance is selected to tune the sensor to the particular, desired frequency of interest. Furthermore, the frame is structured to utilize a desired portion and surface area of the piezoelectric material.

The capacitance of the sensor can generally be expressed by the following relationship: C=εS/D, where C is the sensor's capacitance, ∈ is the dielectric constant associated with the material type selected, S is the surface area of the material, and D is the material thickness (e.g., the distance between the material's conducive layers). In one embodiment, the piezoelectric material (having a predetermined capacitance) is coupled to an sensor impedance (or resistance) to effectively create a high-pass filter having a predetermined high-pass cutoff frequency. The high-pass cutoff frequency is generally the frequency at which filtering occurs. For example, in one embodiment, only frequencies above the cutoff frequency (or above approximately the cutoff frequency) are transmitted.

The amount of charge stored in the conductive layers of the piezoelectric material is generally determined by the thickness of its conductive portions. Therefore, controlling material thickness can control stored charge. One way to control material thickness is to use nanotechnology or MEMS techniques to precisely control the deposition of the electrode layers.

Charge control also leads to control of signal intensity and sensor sensitivity. In addition, as discussed above, mechanical dampening can also be provided by controlling the material thickness to further control signal intensity and sensor sensitivity.

FIG. 33 illustrates an exploded view of the multi-parameter sensor assembly 3100 of FIGS. 31 and 32. As discussed above, the cap sub-assembly 3102 includes a patient adhesive 3108 and a cap 3110, and the sensor sub-assembly 3104 includes a printed circuit board 3114, frame 3116, sensing element 3118, and bonding layer 3120.

In one embodiment, manufacturability of the multi-parameter sensor assembly 100 is improved by combining various components into sub-assemblies. For example, subassemblies simplifies production by allowing a components to be added one at a time, instead of having to combine multiple components at the same time. Additional detail regarding simplified manufacturability of a multi-parameter sensor assembly 3100 is provided below. In addition, subassemblies can be tested during the manufacturing process, which allows defective parts and subassemblies to be identified, repaired, and/or replaced prior to production of a finished good. This saves costs and improves efficiency as well.

In some embodiments, the patient adhesive 3108 is attached to the cap 3110 with a bonding layer (not shown), e.g., a bonding tape. The bonding layer can be double sided and positioned within the cap 3110. Alternatively, in other embodiments, the patient adhesive 3108 is attached to the cap 3110 by fusing, glue, heat staking, etc. In some embodiments, the patient adhesive 3108 includes polyurethane, a co-polymer, polypropylene, mylar, and/or a polymer. The patient adhesive 3108 is generally flexible and pliable, and in some cases, provides a moisture seal. The patient adhesive 3108 is sometimes a fillum, thin sheet, and/or a patch. In one embodiment, the patient adhesive 3108 is a patch that covers the bonding tape only or the bonding tape and the piezo material of the sensor.

In another embodiment, the patient adhesive 3108 is not attached to the patient directly, but is attached to a second patient adhesive, which attaches the sensor 3100 to the patient. The second patient adhesive can have different adhesive characteristics, size, or thickness than the patient adhesive 3108. Providing separate patient adhesives allows the user to select a particular, predetermined adhesive based upon the patient's particular skin type. For example, a particular adhesive could be selected based upon whether the patient's skin is covered with hair, whether the patient has very sensitive skin (e.g., if the patient is a baby, sunburnt, etc.), whether the sensor is to be used during exercise (e.g., during perspiration), or whether the sensor is to be used when the patient is sleeping. The second patient adhesive is sometimes referred to as an auxiliary adhesive. The second patient adhesive could be attached to, or substituted for, the patient adhesive 3108.

In another embodiment, the entire cap assembly 3102 is removable, replaceable, and/or disposable. In some embodiments, the cap assembly 3102 is ultrasonically welded, methylene chloride welded, press fit, and/or snap-in attached to the sensor sub-assembly 3104. Furthermore, the sensor assembly 3100 can be provided in several different sizes, such as about 1 cm×about 2 cm, about 0.5 cm×about 1 cm, or about 2 cm×about 4 cm.

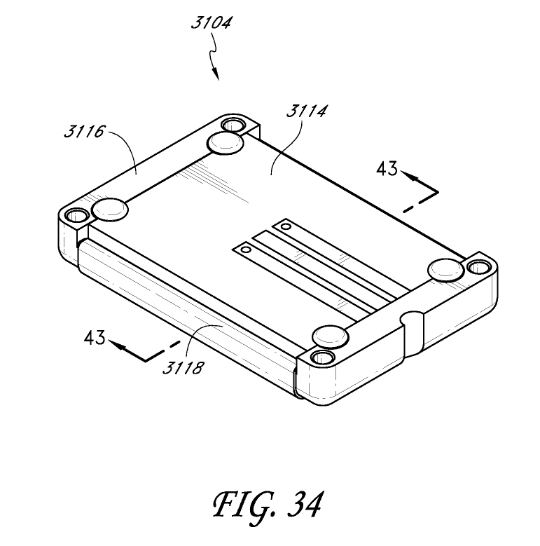

FIG. 34 illustrates a sensor sub-assembly 3104 in accordance with one embodiment of the present invention. The sensor sub-assembly 3104 includes a printed circuit board 3114, a frame 3116, a sensing element 3118, and a bonding layer 3120 (not shown). In one embodiment, the printed circuit board 3114 sits inside of a cavity of the frame 3116 and is pressed against the sensing element 3118 to create a stable electrical contact between the printed circuit board 3114 and electrical contact portions of the sensing element 3118. A bonding layer 3120 is positioned between the frame 3116 and the sensing element 3118, and allows the sensing element 3118 to be held in place with respect to the frame 3116 prior to placement of the printed circuit board 3114. Additional details are provided below.

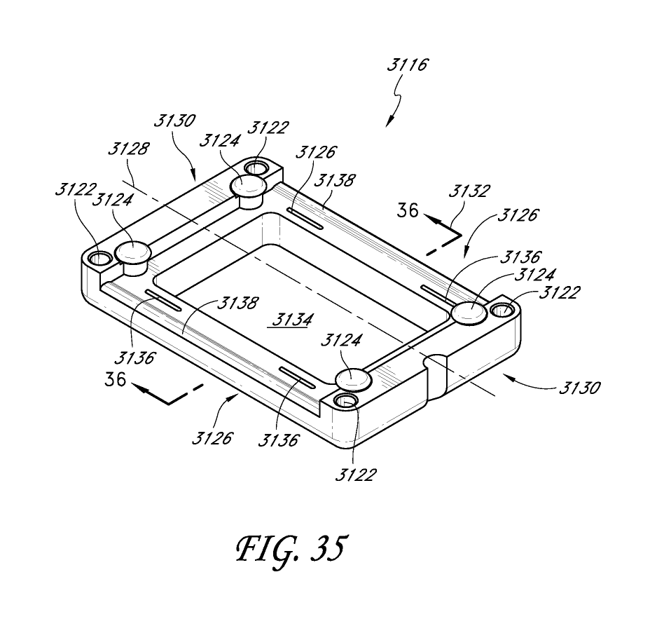

One embodiment of the frame 3116 of the sensor sub-assembly 3104 is shown in greater detail in FIG. 35. The illustrated frame 3116 has a generally rectangular shape, as viewed from the top or bottom, although the frame shape could be any shape, including square, oval, elliptical, elongated, etc. In one embodiment, the frame 3116 has a length of about 10-22 mm. In another embodiment, the frame 3116 has a width of about 8-15 mm. In yet another embodiment, the frame 3116 has a height of about 2-4 mm.

In one embodiment, the frame 3116 includes four guide holes 3122, for example, near each of the frame's 3116 four corners. The guide holes 3122 are generally cylindrical in shape, although in other embodiments they are tapered, conical or frustoconical in shape. The guide holes 3122 provide alignment and mating for corresponding alignment pins (not shown) on the cap 3110. In some embodiments, the inside diameter of the guide holes 3122 is larger than the outside diameter of the alignment pins such that the guide holes do not contact the alignment pins when inserted. In other embodiments, the guide holes 3122 form a press-fit connection with the alignment pins of the cap 3110.

The frame 3116 also includes at least one locking post 3124, which is used to lock the printed circuit board 3114 into the sensor sub-assembly 3104, as described below. In one embodiment, the frame 3116 includes four locking posts 3124, for example, near each of the frame's 3116 four corners. In other embodiments, the frame 3116 includes one, two, or three locking posts 3124.

In one embodiment, the locking posts 3124 are formed from the same material as, and are integral with the frame 3116. When the locking posts 3124 are brought into contact with horns of an ultrasonic welder, they liquefy and flow to form a mushroom-shaped weld over the material directly beneath it. As will be described below, when the components of the sensor sub-assembly 3104 are in place, the locking posts 3124 are flowed to lock all components into a fixed position.

In other embodiments, the locking posts 3124 are not formed from the same material as the frame 3116. For example, in other embodiments, the locking posts 3124 include clips, welds, adhesives, and/or other locks to hold the components of the sensor sub-assembly 3104 in place when the locking posts 3124 are locked into place.

In one embodiment, the frame 3116 includes two frame segments 3126 that extend parallel or substantially parallel to a longitudinal axis 3128 of the frame 3116. The frame 3116 also includes two transverse frame segments 3130 that extend parallel or substantially parallel to a transverse axis 3132 of the frame 3116. A cavity 3134 is defined by the inside surfaces of the frame segments 3126 and transverse frame segments 3130. The cavity 3134 serves as an acoustic chamber of the multi-parameter sensor assembly 3100.

The frame 3116 also includes one or more contact bumps 3136, which press into corresponding contact strips of the printed circuit board 3114 when the sensor sub-assembly 3104 is assembled. The contact bumps 3136 help assure a stable, constant contact resistance between the printed circuit board 3114 and the sensing element 3118, as described in greater detail below with respect to FIG. 39.

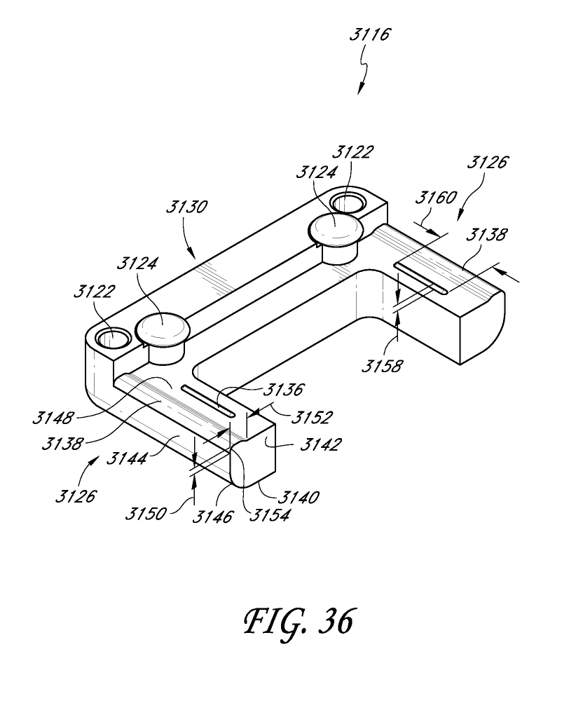

In one embodiment, the frame segments 3126 have a generally or partially, square or rectangular cross-sectional shape, as taken along the transverse axis 3132, as can be seen in greater detail in FIG. 36. The cross-sectional shape of the frame segments 3126 may include one or more rounded corners or raised ridges 3138 or protrusions, as discussed below. The rounded corners and raised ridges 3138 help assure that the sensing element 3118 extends smoothly across the frame 3116, and does not include wrinkles, folds, crimps and/or unevenness. In addition, the dimensions of the rounded corners and raised ridges 3138 control the tension provided to the sensing element 3118 when it is stretched across the frame 3116 in the direction of the transverse axis 3132, as described in greater detail below.

FIG. 36 shows a cross-sectional view of one embodiment of the frame 3116. The patient-contact side 3140 of each frame segment 3126 extends from an inside surface 3142 to an outside surface 3144. The patient-contact side 3140 transitions to the outside surface 3144 via a first curve 3146. The dimensions of the first curve 3146 are selected such that the sensing element 3118 smoothly wraps around the frame 3116 when attached, as discussed above. In one embodiment, the first curve 3146 has a radius of about 1 mm, or is within the range of about 0.5 to 1.5 mm.

The outside surface 3144 transitions to a PCB-contact side 3148 via a raised ridge 3138. The height 3150 and width 3152 of the raised ridge 3138 are defined by a second curve 3154 and a chamfer 3156 of the raised ridge 3138. In one embodiment, the height 3150 is about 0 to 0.70 mm, sometimes about 0.13 mm. In other embodiments, the width 3152 is about 0.67 mm, or in the range of about 0 to 1.5 mm. In some embodiments the second curve 3154 radius is 0.41 mm, 0 to 1.0 mm. In yet other embodiments, the chamfer 3156 extends at an angle of 30 degrees, or 0 to 90 degrees with respect to the PCB-contact side 3148. In the illustrated embodiment, the inside surface 3142 is parallel or substantially parallel to the outside surface 3144, and the patient-contact side 3140 is parallel or substantially parallel to the PCB-contact side 3148.

The contact bumps 3136 are dimensioned to press a portion of the sensing element 3118 into the printed circuit board 3114 when the sensor sub-assembly 3104 is assembled. In one embodiment, the contact bumps 3136 have a height 3158 of about 0.26 mm, or in the range of about 0.2 to 0.3 mm. The height 3158 is generally selected to provide adequate force and pressure between the sensing element 3118 and printed circuit board 3114 as will be discussed in greater detail below.

In one embodiment, the contact bumps 3136 have a triangular cross-sectional shape. The triangular cross-sectional shape allows greater pressure between the sensing element 3118 and printed circuit board 3114. However, in other embodiments, the contact bumps 3136 have a trapezoidal, semi-circular, or semi-elliptical cross-sectional shape. The particular cross-sectional shape may be selected to control the pressure and force between the printed circuit board 3114 and sensing element 3118. By controlling pressure and force, the contact resistance between the two conductive surfaces of the printed circuit board 3114 and sensing element 3118 can be controlled.

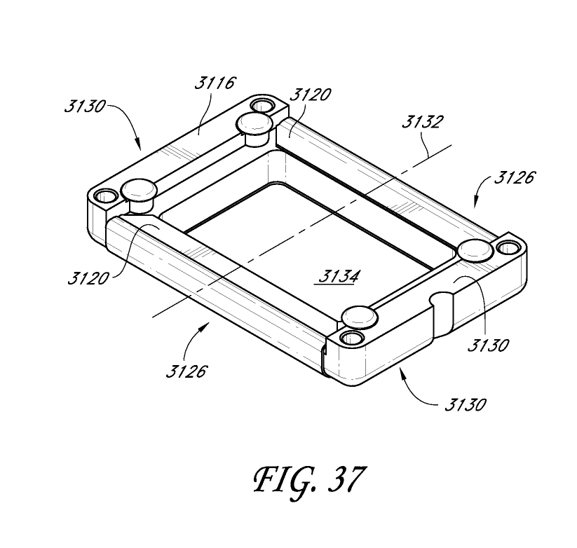

During assembly of the sensor sub-assembly 3104, a bonding layer 3120 is wrapped around the two frame segments 3126 of the frame 3116 in the direction of the transverse axis 3132, as shown in FIG. 37. In some embodiments, the bonding layer 3120 is an elastomer and has adhesive on both of its faces. In other embodiments, the bonding layer 3120 is a rubber, plastic, tape, such as a cloth tape, foam tape, or adhesive film, or other compressible material that has adhesive on both its faces. For example, in one embodiment, the bonding layer 3120 is a conformable polyethylene film that is double coated with a high tack, high peel acrylic adhesive. In many embodiments, the bonding layer 3120 is water resistant or water proof, and provides a water-proof or water-resistant seal. The water-resistant property of the boding layer 3120 provides the advantage of preventing moisture from entering the acoustic chamber or cavity 3134, as discussed in greater detail below. The bonding layer 3120 in some embodiments is about 2, 4, 6, 8 or 10 mil thick. In addition, the bonding layer 3120 also helps prevent inside electrode from shorting to the outside electrode.

The bonding layer 3120 is attached to the PCB-contact side 3148, raised ridges 3138, outside surface 3144, first curve 3146, and patient-contact side 3140 of the frame segments 3126. The bonding layer 3120 is dimensioned such that it also contacts a patient-contact 3140 side of the transverse frame segments 3130. In this manner, the bonding layer 3120 surrounds the opening to the cavity 3134 at the patient-contact side of the frame 3116.

In one embodiment, as shown in FIG. 38, after the bonding layer 3120 is attached to the frame 3116, a sensing element 3118 is attached to the bonding layer 3120. The opposite edges 3158 of the sensing element 3118 extend in the direction of the transverse axis 3132 and wrap over the frame 3116 and bonding layer 3120 towards the longitudinal axis 3128. The edges 3158 of the sensing element 3118 also extend past the contact bumps 3136 of the frame 3116, such that at least a portion of the sensing element 3118 is above each of the contact bumps 3136.

A cross sectional view of the assembly of FIG. 38 is provided in FIG. 39. In the illustrated embodiment, the sensing element 3118 and bonding layer 3120 form a water resistant or water proof seal around the patient-contact surface edge of the cavity 3134. The water resistant seal prevents moisture, such as perspiration, or other fluids, from entering the cavity 3134 of the multi-parameter sensor assembly 3100 when worn by a patient. This is particularly advantageous when the patient is wearing the multi-parameter sensor assembly 3100 during physical activity. The bonding layer 3120 serves as an electrical insulator between the front and back (or first and second) surfaces of the sensing element 3118. In this way, the bonding layer 3120 prevents current flow and/or a conductive path from forming from the first surface of the sensing element 3118 to its second surface as a result of patient perspiration entering and/or contacting the sensing element 3118 and/or sensor assembly 3100.

One embodiment of a piezoelectric sensing element 3118 is provided in FIGS. 40-42. The sensing element 3118 includes a substrate 3160 and coatings 3162, 3164 on each of its two planar faces 3166, 3168. The planar faces 3166, 3168 are substantially parallel to each other. At least one through hole 3170 extends between the two planar faces 3166, 3168. In one embodiment, the sensing element 3118 includes two or three through holes 3170.

In one embodiment, a first coating 3162 is applied to the first planar face 3166, the substrate 3160 wall of the through holes 3170, and a first conductive portion 3172 of the second planar face 3168. By applying a first coating 3162 to the through holes 3170, a conductive path is created between the first planar face 3166 and the first conductive portion 3172 of the sensing element 3118. A second coating 3164 is applied to a second conductive portion 3174 of the second planar face 3168. The first conductive portion 3172 and second conductive portion 3174 are separated by a gap 3176 such that the first conductive portion 3172 and second conductive portion 3174 are not in contact with each other. In one embodiment, the first conductive portion 3172 and second conductive portion 3174 are electrically isolated from one another.

In some embodiments, the first and second conductive portions 3172, 3174 are sometimes referred to as masked portions, or coated portions. The conductive portions 3172, 3174, can be either the portions exposed to, or blocked from, material deposited through a masking, or deposition process. However, in some embodiments, masks aren't used. Either screen printing, or silk screening process techniques can be used to create the first and second conductive portions 3172, 3174.

In another embodiment, the first coating 3162 is applied to the first planar face 3166, an edge portion of the substrate 3160, and a first conductive portion 3172. By applying the first coating 3162 to an edge portion of the substrate 3160, through holes 3170 can optionally be omitted.

In one embodiment, the first coating 3162 and second coating 3164 are conductive materials. For example, the coatings 3162, 3164 can include silver, such as from a silver deposition process. By using a conductive material as a coating 3162, 3164, the multi-parameter sensor assembly 3100 can function as an electrode as well.

Electrodes are devices well known to those of skill in the art for sensing or detecting the electrical activity, such as the electrical activity of the heart. Changes in heart tissue polarization result in changing voltages across the heart muscle. The changing voltages create an electric field, which induces a corresponding voltage change in an electrode positioned within the electric field. Electrodes are typically used with echo-cardiogram (EKG or ECG) machines, which provide a graphical image of the electrical activity of the heart based upon signal received from electrodes affixed to a patient's skin.

Therefore, in one embodiment, the voltage difference across the first planar face 3166 and second planar face 3168 of the sensing element 3118 can indicate both a piezoelectric response of the sensing element 3118, such as to physical aberration and strain induced onto the sensing element 3118 from acoustic energy released from within the body, as well as an electrical response, such as to the electrical activity of the heart. Circuitry within the multi-parameter sensor assembly 3100 and/or within a physiological monitor (not shown) coupled to the multi-parameter sensor assembly 3100 distinguish and separate the two information streams. One such circuitry system is described in U.S. Provisional No. 60/893,853, filed Mar. 8, 2007, titled, “Multi-parameter Physiological Monitor,” which is expressly incorporated by reference herein.

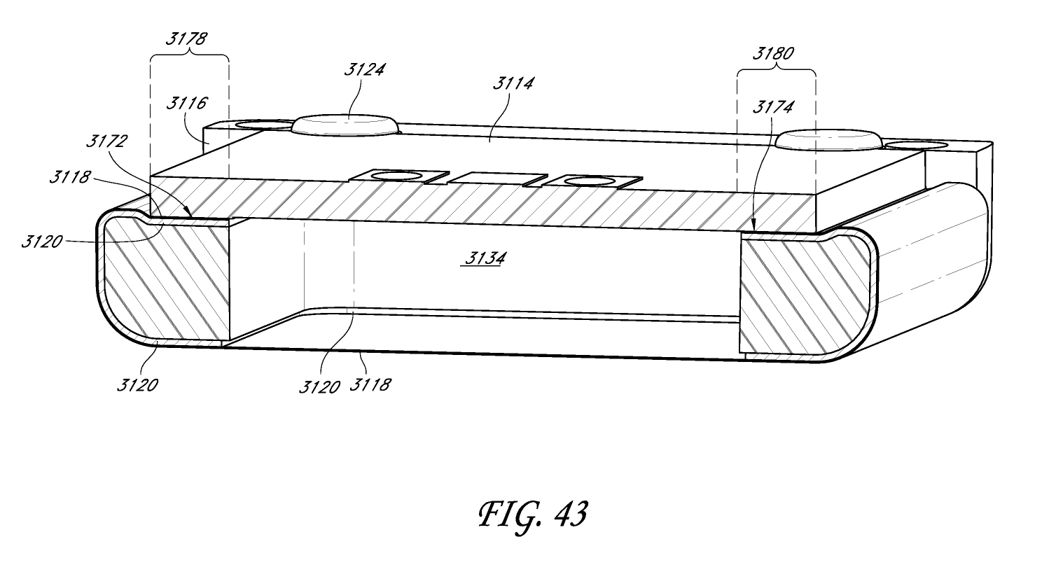

Referring back to FIGS. 40-42, the sensing element 3118 is flexible and can be wrapped at its edges, as shown in FIG. 42. In one embodiment, the sensing element 3118 is wrapped around the frame 3116, as shown in FIG. 39. In addition, by providing both a first conductive portion 3172 and a second conductive portion 3174, both the first coating 3162 and second coating 3164 can be placed into direct electrical contact with the same surface of a printed circuit board 3114, as shown in FIGS. 34 and 43. This provides the advantage of being able to symmetrically place the sensing element 3118 under tension, and avoids uneven stress distribution through the sensing element 3118.

FIG. 43 shows a cross-sectional view of a sensor sub-assembly 3104 in accordance with another embodiment of the present invention. After the sensing element 3118 and bonding layer 3120 are attached to the frame 3116, a printed circuit board 3114 is then provided. The printed circuit board 3114 is placed on top of the sensing element 3118 such that a first edge 3178 of the printed circuit board 3114 is placed over the first conductive portion 3172 of the sensing element 3118, and a second edge 3180 of the printed circuit board 3114 is placed over the second conductive portion 3174 of the sensing element 3118.

The printed circuit board 3114 is pressed down into the sensing element 3118 in the direction of the frame 3116. As the printed circuit board 3114 is pressed downward, the contact bumps 3136 of the frame 3116 push the bonding layer 3120 and sensing element 3118 into contact strips located along the first and second sides or edges 3176, 3178 of the printed circuit board 3114. The contact strips of the printed circuit board 3114 are made from conductive material, such as gold. Other materials having a good electronegativity matching characteristic to the conductive portions 3172, 3174 of the sensing element 3118, may be used instead. The elasticity or compressibility of the bonding layer 3120 acts as a spring, and provides some variability and control in the pressure and force provided between the sensing element 3118 and printed circuit board 3114.

Once the desired amount of force is applied between the printed circuit board 3114 and frame 3116, the locking posts 3124 are vibrated or ultrasonically welded until the material of the locking posts 3124 flows over the printed circuit board 3114. The locking posts 3124 can be welded using any of a variety of techniques, including heat staking, or placing ultrasonic welding horns in contact with a surface of the locking posts 3124, and applying ultrasonic energy. Once welded, the material of the locking posts 3124 flows to a mushroom-like shape, hardens, and provides a mechanical restraint against movement of the printed circuit board 3114 away from the frame 3116 and sensing element 3118. By mechanically securing the printed circuit board 3114 with respect to the sensing element 3118, the various components of the sensor sub-assembly 3104 are locked in place and do not move with respect to each other when the multi-parameter sensor assembly 3100 is placed into in clinical use. This prevents the undesirable effect of inducing electrical noise from moving assembly components or inducing instable electrical contact resistance between the printed circuit board 3114 and the sensing element 3118.

Therefore, the printed circuit board 3114 can be electrically coupled to the sensing element 3118 without using additional mechanical devices, such as rivets or crimps, conductive adhesives, such as conductive tapes or glues, like cyanoacrylate, or others. In addition, the mechanical weld of the locking posts 3124 helps assure a stable contact resistance between the printed circuit board 3114 and the sensing element 3118.

The contact resistance between the sensing element 3118 and printed circuit board 3114 can be measured and tested by accessing test pads on the printed circuit board 3114. For example, in one embodiment, the printed circuit board 3114 includes three discontinuous, aligned test pads that overlap two contact portions between the printed circuit board 3114 and sensing element 3118. A drive current is applied, and the voltage drop across the test pads is measured. For example, in one embodiment, a drive current of about 100 mA is provided. By measuring the voltage drop across the test pads the contact resistance can be determined by using Ohm's law, namely, voltage drop (V) is equal to the current (I) through a resistor multiplied by the magnitude of the resistance (R), or V=IR.

The printed circuit board 3114 includes various electronic components mounted to either or both faces of the printed circuit board 3114. When the multi-parameter sensor assembly 3100 is assembled, the electronic components of the printed circuit board 3114 may extend into the assembly's cavity 3134 or acoustic chamber. To reduce space requirements and to prevent the electronic components from adversely affecting operation of the multi-parameter sensor assembly 3100, the electronic components can be low-profile, surface mounted devices. The electronic components are often connected to the printed circuit board 3114 using conventional soldering techniques, for example the flip-chip soldering technique. Flip-chip soldering uses small solder bumps such of predictable depth to control the profile of the soldered electronic components.

In some embodiments, the electronic components include filters, amplifiers, etc. for pre-processing or processing a low amplitude electric signal received from the sensing element 3118, prior to transmission through a cable to a physiological monitor. In other embodiments, the electronic components include a processor or pre-processor to process electric signals. Such electronic components may include, for example, analog-to-digital converters for converting the electric signal to a digital signal and a central processing unit for analyzing the resulting digital signal.

In one embodiment, the printed circuit board 3114 also includes a wireless transmitter, thereby eliminating mechanical connectors and cables. For example, optical transmission via at least one optic fiber or radio frequency (RF) transmission is implemented in other embodiments. In other embodiments, the sensor assembly 3100 includes a security device, such as an information element, to assure compatibility and between the sensor assembly 3100 and the physiological monitor to which it is attached. In addition, the sensor assembly 3100 can include any of a variety of information storage devices, such as readable and/or writable memories. Information storage devices can be used to keep track of device usage, manufacturing information, duration of sensor usage, other sensor, physiological monitor, and/or patient statistics, etc.

In other embodiments, the printed circuit board 3114 includes a frequency modulation circuit having an inductor, capacitor and oscillator, such as that disclosed in U.S. Pat. No. 6,661,161, which is incorporated by reference herein. In another embodiment, the printed circuit board 3114 includes an FET transistor and a DC-DC converter or isolation transformer and phototransistor. Diodes and capacitors may also be provided. In yet another embodiment, the printed circuit board 3114 includes a pulse width modulation circuit.

In yet another embodiment, the printed circuit board 3114 includes an information element that communicates calibration and/or identification information to a physiological monitor. For example, in one embodiment, the information element identifies the manufacturer, lot number, expiration date, and/or other manufacturing information. In another embodiment, the information element includes calibration information regarding the multi-parameter sensor assembly 3100.

In one embodiment, the information element includes an EPROM, EEPROM, ROM, or other readable memory device. Information from the information element is provided to the physiological monitor according to any communication protocol known to those of skill in the art. For example, in one embodiment, information is communicated according to an I2C protocol. U.S. Provisional No. 60/893,850, filed Mar. 8, 2007, titled “Backward Compatible Physiological Sensor,” which is incorporated by reference herein, teaches various methods of communicating information from an information element in a multi-parameter sensor assembly 3100 to a physiological monitor.

The information element may be provided on or in electrical communication with the printed circuit board 3114. In one embodiment, the information element is provided on a cable connected to the printed circuit board.

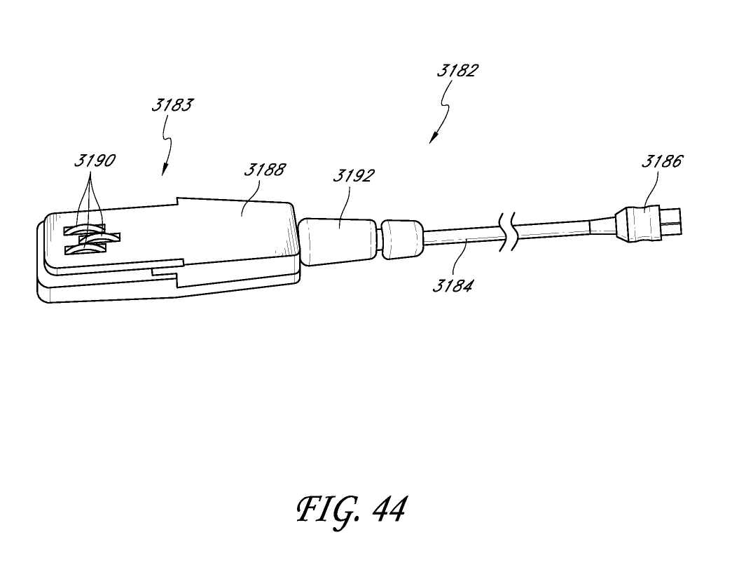

FIG. 44 shows one embodiment of a cable assembly 3182 configured to couple the multi-parameter sensor assembly 3100 to a physiological monitor. The cable assembly 3182 includes a sensor connector 3183, a cable 3184 or lead, and a physiological monitor connector 3186. The cable 3184 typically carries three conductors within a shielding: one conductor to provide power to the multi-parameter sensor assembly 3100, one conductor to provide a ground signal to the multi-parameter sensor assembly 3100, and one conductor to transmit signals from the multi-parameter sensor assembly 3100 to the physiological monitor. In some embodiments the cable 3184 carries two conductors within a shielding, and the shielding layer acts as the ground conductor. In other embodiments, the cable assembly 3182 includes three or more conductors, such as four conductors. For example, in one embodiment, the cable assembly 3182 includes the three conductors listed above as well as an additional conductor for a secondary signaling lead. In some embodiments, the “ground signal” is an earth ground, but in other embodiments, the “ground signal” is a patient ground, sometimes referred to as a patient reference, a patient reference signal, a return, or a patient return.

In one embodiment, the sensor connector 3183 includes a housing 3188, which can be made from an electrically insulating molded plastic material. The housing 3188 encloses three contacts 3190, such as electrically conductive spring blades 3190 for contacting the three laterally adjacent electrically conductive traces of the printed circuit board 3114. The contacts 3190 are also electrically connected to the conductors of the cable 3184. Although the illustrated embodiment is shown having three contacts 3190, the housing 3188 can include three or more contracts 3190. In one embodiment, the housing 3188 includes four contacts.

A pliable plastic cuff 3192 is mounted on the cable 3184 adjacent the housing 3188. In one embodiment, the cuff 3192 improves the durability of the cable 3184 by acting as a strain relief. A physiological monitor connector 3186 is mounted to the opposite end of the cable 3184 and provides connectivity to a physiological monitor. When connected to the multi-parameter sensor assembly 3100, the spring blades 3190 and the electrical conductors of the cable 3184 are placed in electrical communication with the printed circuit board 3114 of the multi-parameter sensor assembly 3100.

In other embodiments, the cable 3184 includes two disconnectable portions, each having a different stiffness or flexibility. For example, in one embodiment, the cable 3184 includes a monitor cable portion and a sensor cable portion. The sensor assembly 3100 attaches to the sensor cable, the sensor cable attaches to the monitor cable, and the monitor cable attaches to a physiological monitor.

The sensor cable portion is more flexible and lighter than the monitor cable. In one embodiment, the sensor cable is about 6″ long. The sensor cable can be selected to minimize tribology and to be less sensitive to physical movement or disturbance. The sensor cable can be secured to the patient, e.g., by tape, at about 6″ to 80″ from the sensor 3100. A connector at the end of the sensor cable is configured to connect to a mating connector located at the end of the monitor cable.

The monitor cable is stiffer, stronger, heavier, and/or more mechanically and/or electrically reinforced/shielded than the sensor cable. In some embodiments, the monitor cable is about 4′ to about 8′ long. The sensor cable can permanently or removably attached (e.g., snapped or fused) to the monitor cable.

In one embodiment, the connector 3186 is compliant with international standard IEC-60601-1. The connector 3186 can include a key lock, over-molded connector, and/or sealed pins to prevent water ingress. In one embodiment, the connector 3186 is the #220 connector manufactured by PlasticsOne, Inc.

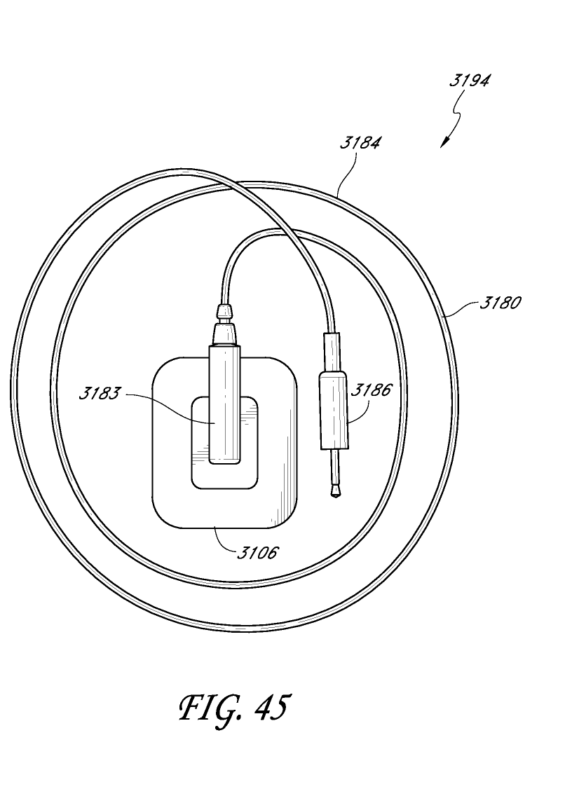

FIG. 45 is a top perspective of a sensor system 3194 in accordance with yet another embodiment of the present invention. The sensor system 3194 includes a multi-parameter sensor assembly, such as the multi-parameter sensor assembly 3100 of FIGS. 31-33 coupled to a cable assembly, such as the cable assembly of FIG. 44.

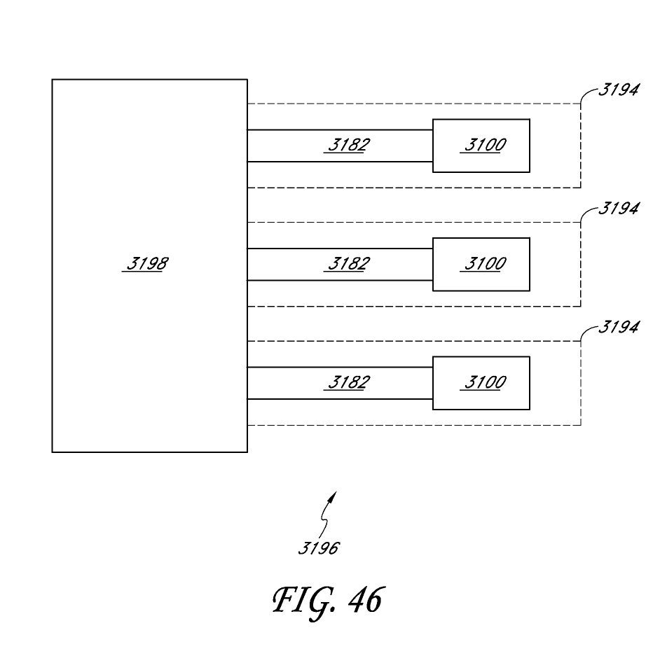

FIG. 46 is a block diagram of one embodiment of a physiological monitoring system 3196, which includes a physiological monitor 3198 coupled to three sensor systems, such as the sensor system 3194 of FIG. 45. The physiological monitoring system 3196 can be coupled to any number of sensor systems 3194 as desired. When monitoring ECG and bio-acoustic sounds, it may be desirable to include two sensor systems 3194 and one ECG lead instead of three sensor systems 3194.

For example, in one embodiment, the physiological monitoring system 3196 includes a first sensor system 3194 that is positioned near a patient's trachea. The first sensor system 3194 is configured to detect respiratory sounds of the patient, as perceived through the patient's neck and trachea. The first sensor system 3194 is also configured to perform as an ECG electrode, as described above.

The second sensor system 3194 is positioned near the patient's heart. The second sensor system 3194 is configured to detect cardiac sounds of the patient, as perceived through the patient's chest. The second sensor system 3194 is also configured to perform as an ECG electrode, as described above.

The third sensor system includes only an ECG electrode. For example, the third sensor system may not include a piezoelectric sensing element 3118 as provided with the first and second sensor systems 3194. The third sensor system is therefore configured only to perform as an ECG electrode. A complete ECG signal of the patient may be constructed from the relative voltage levels provided by the three sensor systems 3194. Additional or fewer sensor systems may be provided with the physiological monitoring system 3196.

The physiological monitoring system 3196 is sometimes referred to as an acoustic signal processing system, and is configured to measure and/or determine any of a variety of physiological parameters of a medical patient. For example, in various embodiments, the physiological monitoring system 3196 is an acoustic respiratory monitor. An acoustic respiratory monitor can determine any of a variety of respiratory parameters of a patient, including respiratory rate, inspiratory time, expiratory time, inspiration-to-expiration ratio, inspiratory flow, expiratory flow, tidal volume, minute volume, apnea duration, breath sounds, rales, rhonchi, stridor, and changes in breath sounds such as decreased volume or change in airflow. In addition, in some cases the acoustic signal processing system monitors other physiological sounds, such as heart rate to help with probe off detection, heart sounds (e.g., S1, S2, S3, S4, and murmurs), and change in heart sounds such as normal to murmur or split heart sounds indicating fluid overload. Moreover, the acoustic signal processing system may use a second probe over the chest for better heart sound detection, keep the user inputs to a minimum (example, height), and use an HL7 interface to automatically input demography.

Finally, in other embodiments, the physiological monitoring system 3196 includes a photoplethysmograph sensor configured to determine the blood-oxygen concentration of the patient.

In addition to those processes described above, other processes and combination of process will be apparent to those of skill in the art. In addition, those of skill in the art will appreciate that although certain embodiments of the present invention have been described in relation to operational amplifiers, other circuit components could be used in place of operational amplifiers. For example, transistor amplifiers and other forms of amplifiers could be used in place of the operational amplifiers described herein. Likewise, filters and circuit components disclosed herein may be interchanged with other filters and circuit components. Moreover, amplifiers in one or more gain stages may be removed entirely.

Those of skill in the art will understand that the information and signals discussed herein can be represented using any of a variety of different technologies and techniques. For example, data, instructions, commands, information, signals, bits, symbols, and chips that can be referenced throughout the above description can be represented by voltages, currents, electromagnetic waves, magnetic fields or particles, optical fields or particles, or any combination thereof.

Those of skill will further appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans can implement the described functionality in varying ways for each particular application, but such implementation decisions should not be interpreted as causing a departure from the scope of the present invention.

The various illustrative logical blocks, modules, and circuits described in connection with the embodiments disclosed herein can be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, conventional processor, controller, microcontroller, state machine, etc. A processor can also be implemented as a combination of computing devices, e.g., a combination of a DSP and a microprocessor, a plurality of microprocessors, one or more microprocessors in conjunction with a DSP core, or any other such configuration. In addition, the term “processing” is a broad term meant to encompass several meanings including, for example, implementing program code, executing instructions, manipulating signals, filtering, performing arithmetic operations, and the like.

The steps of a method or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, a DVD, or any other form of storage medium known in the art. A storage medium is coupled to the processor such that the processor can read information from, and write information to, the storage medium. In the alternative, the storage medium may be integral to the processor. The processor and the storage medium can reside in an ASIC. The ASIC can reside in a user terminal. In the alternative, the processor and the storage medium can reside as discrete components in a user terminal.

The modules can include, but are not limited to, any of the following: software or hardware components such as software object-oriented software components, class components and task components, processes, methods, functions, attributes, procedures, subroutines, segments of program code, drivers, firmware, microcode, circuitry, data, databases, data structures, tables, arrays, or variables.

In addition, although this invention has been disclosed in the context of a certain preferred embodiments, it will be understood by those skilled in the art that the present invention extends beyond the specifically disclosed embodiments to other alternative embodiments and/or uses of the invention and obvious modifications and equivalents thereof. For example, while the present signal processing systems and methods have been described in the context of particularly preferred embodiments, the skilled artisan will appreciate, in view of the present disclosure, that certain advantages, features and aspects of the signal processing systems, devices, and methods may be realized in a variety of other applications and software systems.

Although the foregoing invention has been described in terms of certain preferred embodiments, other embodiments will be apparent to those of ordinary skill in the art from the disclosure herein. For example, a person of ordinary skill will recognize from the disclosure herein that there are various different types of information elements and various different ways to communicate with the information element that can be used with the sensor of the present disclosure. As another example, a person of ordinary skill will recognize from the disclosure herein that various different power storage devices can be used with the sensor of the present disclosure. Additionally, other combinations, omissions, substitutions and modifications will be apparent to the skilled artisan in view of the disclosure herein. It is contemplated that various aspects and features of the invention described can be practiced separately, combined together, or substituted for one another, and that a variety of combination and subcombinations of the features and aspects can be made and still fall within the scope of the invention. Furthermore, the systems described above need not include all of the modules and functions described in the preferred embodiments. Accordingly, the present invention is not intended to be limited by the recitation of the preferred embodiments, but is to be defined by reference to the appended claims.

What is claimed is:

1. A physiological sensor configured to obtain physiological information from a patient and transmit the information to a physiological monitor, the physiological sensor comprising:
a conductor configured to provide a communication path for communicating with a physiological monitor;
a power supply configured to receive and store power from the conductor in a first mode and further configured to release the stored power in a second mode; and
sensing circuitry configured to receive power from the conductor in the first mode and to receive power from the power supply in the second mode.

2. The physiological sensor of claim 1, wherein the sensing circuitry is further configured to obtain and communicate physiological information to the physiological monitor in the first and second modes.

3. The physiological sensor of claim 2, further comprising a second communication path configured to communicate the physiological information to the physiological monitor.

4. The physiological sensor of claim 1, further comprising an information element configured to communicate with the monitor through the first conductor in the second mode.

5. The physiological sensor of claim 1, wherein the sensing circuitry comprises acoustic monitoring circuitry.

6. The physiological sensor of claim 5, wherein the acoustic monitoring circuitry comprises a piezoelectric element.

7. The physiological sensor of claim 1, wherein the power supply comprises at least one capacitor.

8. The physiological sensor of claim 1, wherein the information element comprises an EPROM.

9. The physiological sensor of claim 8, wherein the information element stores one or more of a sensor type, a manufacturer, a model number, a serial number, a patient type, manufacturing tolerances, acoustic sensitivity, voltage information, current information, gain, an expiration date, an age of the sensor, use information, and patient information.

10. A method of communicating with a physiological sensor comprising:
supplying power through a first conductor in a first mode to a physiological sensor, wherein the physiological sensor comprises an information element, a power supply configured to receive and store power from the first conductor in the first mode, and sensing circuitry configured to receive power from the first conductor in the first mode; and
communicating with the information element through the first conductor in a second mode, wherein the power supply releases the stored power to the sensing circuitry in the second mode.

11. The method of claim 10, wherein communicating with the information element in the second mode further comprises communicating with the information element using a communication protocol.

12. The method of claim 10, wherein communicating with the information element in the second mode further comprises reading information from the information element.

13. The method of claim 10, wherein communicating with the information element in the second mode further comprises writing information to the information element.

14. The method of claim 10, wherein the sensing circuitry comprises acoustic monitoring circuitry.

15. A method of communicating with a physiological monitor using an attachment comprising:
receiving power at a physiological monitor attachment from a physiological monitor via a conductive path during a first operating mode;
receiving a communication request at the physiological monitor attachment from the physiological monitor via the conductive path to initiate a second operating mode;
communicating with the physiological monitor using the physiological monitor attachment via the conductive path during the second operating mode.

16. The method of claim 15, further comprising providing power to the physiological monitor attachment from a secondary internal power source.

17. The method of claim 15, wherein the physiological monitor attachment comprises a physiological sensor.

18. The method of claim 15, wherein the physiological monitor attachment comprises a cable.

19. The method of claim 15, wherein communicating with the physiological monitor comprises sending information stored in an information element.

20. The method of claim 15, wherein communication with the physiological monitor comprises receiving information from the physiological monitor.
